(19) United States
(12) Patent Application Publication
(10) Pub. No.: US 2013/0338092 A1
(43) Pub. Date: Dec. 19, 2013

(54) COMPOUNDS AND METHODS FOR TARGETING LEUKEMIC STEM CELLS

(71) Applicants: The Broad Institute, Inc., Cambridge, MA (US); The General Hospital Corporation, Boston, MA (US); Dana-Farber Cancer Institute, Inc., Boston, MA (US); The Brigham and Women's Hospital, Inc., Boston, MA (US); President and Fellows of Harvard College, Cambridge, MA (US); Sloan-Kettering Institute for Cancer Research, New York, NY (US)

(72) Inventors: Kimberly Hartwell, Brookline, MA (US); Malcolm A.S. Moore, New York, NY (US); David T. Scadden, Weston, MA (US); Stuart L. Schreiber, Boston, MA (US); Todd R. Golub, Newton, MA (US); Benito Munoz, Newtonville, MA (US); Benjamin L. Ebert, Brookline, MA (US); Andrew M. Stern, Boston, MA (US); Peter G. Miller, Boston, MA (US); D. Gary Gilliland, Wellesley, MA (US); Anne Van Dyk Carpenter, Ashland, MA (US); David J. Logan, Maynard, MA (US); Joseph Negri, Jamaica Plain, MA (US); Nicola Tolliday, Jamaica Plain, MA (US); Alykhan Shamji, Somerville, MA (US); Siddhartha Mukherjee, New York, NY (US); Alison Stewart, Cambridge, MA (US)

(21) Appl. No.: 13/969,213

(22) Filed: Aug. 16, 2013

Related U.S. Application Data
(63) Continuation of application No. PCT/US2012/025745, filed on Feb. 17, 2012.
Application No. PCT/US2012/025745 is a continuation of application No. PCT/US2012/025743, filed on Feb. 17, 2012.
(60) Provisional application No. 61/444,701, filed on Feb. 19, 2011.
(60) Provisional application No. 61/444,701, filed on Feb. 19, 2011.

(51) Int. Cl. G01N 33/50; A61K 31/4184; A61K 31/395; A61K 31/428; A61K 31/423; A61K 31/704; A61K 31/4745; A61K 31/405; A61K 31/4418; A61K 31/366; A61K 31/22; A61K 31/167; A61K 31/444

(52) U.S. Cl. CPC G01N 33/5011; A61K 31/167; A61K 31/4184; A61K 31/395; A61K 31/428; A61K 31/423; A61K 31/444; A61K 31/4745; A61K 31/405; A61K 31/4418; A61K 31/366; A61K 31/22; A61K 31/704
USPC 514/34; 506/10; 514/616; 514/395; 514/622; 514/449; 514/367; 514/375; 514/332; 514/619; 514/294; 514/419; 514/277; 514/460; 514/510

(57) ABSTRACT

This invention relates to high-throughput, semi-automated methods for identifying compounds that are effective in targeting leukemia stem cells, as well as compounds identified by those methods and uses thereof for treating leukemia.

### CLAIM OF PRIORITY

This application is a continuation of PCT International Application No. PCT/US2012/025745, filed on Feb. 17, 2012, which claims the benefit of U.S. Provisional Patent Application Ser. No. 61/444,701, filed on Feb. 19, 2011, and PCT International Application No. PCT/US2012/025743, filed on Feb. 17, 2012, which also claims the benefit of U.S. Provisional Patent Application Ser. No. 61/444,701. The entire contents of the foregoing are hereby incorporated by reference.

### FEDERALLY SPONSORED RESEARCH OR DEVELOPMENT

This invention was made with Government support under Grant No. R01 GM089652 awarded by the National Institutes of Health. The Government has certain rights in the invention.

### TECHNICAL FIELD

This invention relates to high-throughput, semi-automated methods for identifying compounds that are effective in targeting leukemia stem cells, as well as compounds identified by those methods and uses thereof for treating leukemia.

### BACKGROUND

Leukemia stem cells (LSCs), a subpopulation of leukemia cells capable of self-renewal, have been implicated in disease initiation, poor response to therapy, and clinical outcome (Lapidot, T., Sirard, C., Vormoor, J., Murdoch, B., Hoang, T., Caceres-Cortes, J., Minden, M., Paterson, B., Caligiuri, M. A., and Dick, J. E. (1994). Nature 367, 645-648.; Krivtsov, A. V., Twomey, D., Feng, Z., Stubbs, M. C., Wang, Y., Faber, J., Levine, J. E., Wang, J., Hahn, W. C., Gilliland, D. G., et al. (2006). Nature 442, 818-822.; Cortes, J. E., O'Brien, S. M., Giles, F., Alvarez, R. H., Talpaz, M., and Kantarjian, H. M. (2004). Hematol Oncol Clin North Am 18, 619-639, ix.). The biological similarity between LSCs and normal hematopoietic stem and progenitor cells (HSPCs) further complicates clinical intervention in leukemia by limiting therapeutic opportunity (Eppert, K., Takenaka, K., Lechman, E. R., Waldron, L., Nilsson, B., van Galen, P., Metzeler, K. H., Poeppl, A., Ling, V., Beyene, J., et al. (2011). Nat Med 17, 1086-1093.; Krivtsov, A. V., Twomey, D., Feng, Z., Stubbs, M. C., Wang, Y., Faber, J., Levine, J. E., Wang, J., Hahn, W. C., Gilliland, D. G., et al. (2006). Nature 442, 818-822.). A number of frontline treatments in acute myeloid leukemia (AML) elicit a toxicity toward normal HPSCs that is dose-limiting, such as Daunorubicin and Ara-C. Thus, it is likely that AML treatments able to result in a durable clinical response will selectively target the LSC population.

### SUMMARY

The present invention is based, at least in part, on the development of methods for identifying compounds that affect self-renewal of stem cells, e.g., cancer stem cells, e.g., primary LSC-enriched cellular material in a supportive stromal microenvironment with the examination of a biologically-relevant readout, e.g., cobblestoning. Cobblestoning is the presence of “phase dark” cellular areas located beneath the stromal monolayer (this accounts for their “dark” appearance under phase contrast microscopy), that are associated with self-renewal.

Thus, in one aspect, the invention provides methods for identifying a candidate compound for the treatment of leukemia. The methods include providing a test sample comprising a co-culture of stromal cells and primary leukemic hematopoietic cells; contacting the test sample with a test compound, and maintaining the co-culture for a time and under conditions sufficient for the primary leukemic hematopoietic cells to form areas of cobblestoning; obtaining one or more images of the test sample; detecting areas of cobblestoning in the images of the test sample by applying a classifier to the images, wherein the classifier comprises a set of rules that are executable to identify areas of cobblestoning; and comparing the areas of cobblestoning in a test sample in the presence of the test compound to areas of cobblestoning in a test sample in the absence of the test compound (e.g., in the presence of a carrier-only control), and selecting as a candidate compound a test compound that reduces areas of cobblestoning.

In some embodiments, providing the co-culture includes plating a population of stromal cells in a culture dish; and adding a population of primary hematopoietic stem cells in the same culture dish.

In some embodiments, the methods include providing a control sample comprising a co-culture of stromal cells and normal primary hematopoietic cells; contacting the control sample with a test compound, and maintaining the co-culture for a time and under conditions sufficient for the normal hematopoietic cells to form areas of cobblestoning; obtaining one or more images of the control sample; detecting areas of cobblestoning in the images of the control sample by applying the classifier to the images of the control sample; and comparing the areas of cobblestoning in a control sample in the presence of the test compound to areas of cobblestoning in a control sample in the absence of the test compound, and selecting as a candidate compound a test compound that reduces areas of cobblestoning in the test sample but does not reduce areas of obblestoning in the control sample.

In another aspect, the invention provides methods for identifying a candidate compound for the treatment of leukemia. The methods include providing a test sample comprising a culture of stromal cells; contacting the test sample with a test compound; optionally removing substantially all of the test compound from the test sample; adding a population of primary leukemic hematopoietic cells to the test sample, to form a co-culture, and maintaining the co-culture for a time and under conditions sufficient for the primary leukemic hematopoietic cells to form areas of cobblestoning; obtaining one or more images of the test sample; detecting areas of cobblestoning in the images of the test sample; comparing the areas of cobblestoning in a test sample in the presence of the test compound to areas of cobblestoning in a test sample in the absence of the test compound, and selecting as a candidate compound a test compound that reduces areas of cobblestoning.

In some embodiments, the methods include providing a control sample comprising a culture of stromal cells; contacting the control sample with a test compound; optionally removing substantially all of the test compound from the control sample; adding a population of normal primary hematopoietic cells to the control sample, to form a co-culture, and maintaining the co-culture for a time and under conditions sufficient for the normal primary hematopoietic cells to form areas of cobblestoning; obtaining one or more images of the test sample; detecting areas of cobblestoning in the images of the control sample; comparing the areas of cobblestoning in a control sample in the presence of the test compound to areas of cobblestoning in a control sample in the absence of the test compound, and selecting as a candidate compound a test compound that reduces areas of cobblestoning in the test sample but does not reduce areas of cobblestoning in the control sample.

In some embodiments, detecting areas of cobblestoning in the images of the test sample is performed by applying a classifier to the images, wherein the classifier comprises a set of rules that are executable to identify areas of cobblestoning.

In another aspect, the invention provides methods performed by one or more processing devices. The methods include accessing training data, wherein the training data comprises one or more items of data classified as exhibiting a feature associated with self-renewal of leukemia stem cells (LSCs); generating, from the training data, a classifier, wherein the classifier is configured to classify items of data to a group associated with the feature; applying the classifier to unclassified data; generating, based on applying, one or more classifications of the unclassified data; receiving data indicative of an accuracy of the one or more classifications; and training the classifier with the data received.

In some embodiments, the feature comprises cobblestoning; wherein the classifier comprises a plurality of rules that characterize cellular features that are indicative of cobblestoning.

In some embodiments, the methods also include receiving data indicative of one or more features of a combination of a compound and one or more cells; applying the classifier to the data indicative of the one or more features; classifying the data indicative of the one or more features to the group associated with cobblestoning; and identifying, based on classifying, the compound as affecting self-renewal of LSCs.

In some embodiments, an assay comprises the combination of the compound and the one or more cells.

In some embodiments, the one or more cells comprise stromal cells and primary hematopoietic cells. In some embodiments, the primary hematopoietic cells are primary leukemic hematopoietic cells. In some embodiments, the primary leukemic hematopoietic cells are enriched for leukemic stem cells. In some embodiments, the stromal cells are primary cells or from an immortalized cell line.

In some embodiments, training includes applying an interactive machine learning algorithm to the classifier and the data received.

In some embodiments, the actions of applying, generating the one or more classifications of the unclassified data, receiving and training are performed until the classifier exhibits at least a pre-defined level of accuracy.

In some embodiments, the classifier comprises a set of rules that are executable to identify cobblestoning in an item of data.

In some embodiments, the methods include identifying one or more patterns in the training data, wherein the one or more patterns are indicative of cobblestoning; wherein generating the classifier includes generating one or more rules that categorize the one or more patterns.

In some embodiments, the one or more items of data comprise one or more raw images of cells.

In some embodiments, the rules include one or more of: Cell objects that that have greater than a selected percentage of their perimeter touching other objects; Cell objects with low texture feature (Gabor wavelet) at a 3 pixel scale in the DsRed channel; Cell objects with fewer than a selected number of neighbor objects (within 2 pixels); Cell objects with low texture contrast at a 3 pixel scale in the DsRed channel; Cell objects with high minimum intensity in DsRed channel greater than a selected amount; Cell objects standard deviation in DsRed channel less than a selected amount; Cell objects with low minimum intensity in Stromal channel less than a selected amount; Cell objects with greater than a selected number of neighbor objects (within 2 pixels); Cell objects with a 9th order Zernike shape feature greater than a selected level; Cell objects with a low texture feature (Sum of Entropy) at a 1 pixel scale in the DsRed channel.

In some embodiments, the compound inhibits self-renewal of LSCs.

In some embodiments, the methods include identifying the compound as a candidate compound for promoting treatment of leukemia.

Based on the discovery of novel compounds for targeting leukemic stem cells, provided herein are methods for treating leukemia in a patient using the same.

Provided herein are methods for treating leukemia in a patient, the method comprising: identifying the patient as being in remission; and administering to the patient a therapeutically effective amount of one or more statins, or a prodrug, acid, or form thereof. Also provided herein are methods for reducing the number of leukemia cells in a patient, the method comprising: identifying the patient as being in remission; and administering to the patient a therapeutically effective amount of one or more statins, or a prodrug, acid, or form thereof. This disclosure further provides a method for inhibiting growth of leukemia cells in a patient, the method comprising: identifying the patient as being in remission; and administering to the patient a therapeutically effective amount of statins, or a prodrug, acid, or form thereof. Further provided herein is a method for treating leukemia in a patient, the method comprising: administering to the patient a therapeutically effective amount of one or more statins, or a prodrug, acid, or form thereof, and at least one additional treatment for leukemia. For example, the at least one additional treatment for leukemia can be selected from the group consisting of chemotherapy and irradiation.

In some embodiments, the statins are selected from the group consisting of: atorvastatin, cerivastatin, fluvastatin, lovastatin, mevastatin, pitavastatin, pravastatin, rosuvastatin, simvastatin, and acid forms thereof. For example, the statin can be selected from the group consisting of: cerivastatin, fluvastatin, and acid forms thereof. In some embodiments, the statin is fluvastatin or an acid form thereof.

In some embodiments, the leukemia is selected from the group consisting of: chronic eosinophilic leukemia (CEL), chronic neutrophilic leukemia (CNL), chronic myelogenous leukemia (CML), chronic myelomonocytic leukemia (CMML), hairy cell leukemia (HCL), and chronic lymphocytic leukemia (CLL); acute leukemias include acute myelogenous leukemia (AML) and acute lymphocytic leukemia (ALL).

In some embodiments, the leukemia cells are leukemia stem cells.

In some embodiments, the one or more statins are administered in combination with another chemotherapeutic agent.

This disclosure also provides a method for treating leukemia in a patient, the method comprising administering to the patient a therapeutically effective amount of a compound of formula (I):

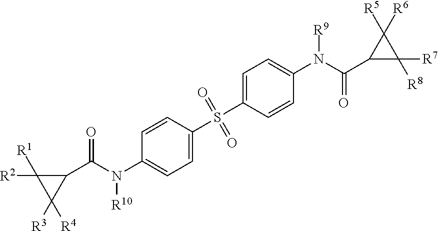

or a pharmaceutically acceptable salt form thereof,

wherein:
R¹, R², R³, R⁴, R⁵, R⁶, R⁷, and R⁸ are independently selected from the group consisting of: hydrogen, C₁₋₆ alkyl, C₁₋₆ alkenyl, and C₁₋₆ alkynyl; and
R⁹ and R¹⁰ are independently selected from the group consisting of: hydrogen and C₁₋₆ alkyl.

Also provided herein is a method for reducing the number of leukemia cells in a patient, the method comprising administering to the patient a therapeutically effective amount of a compound of formula (I) or a pharmaceutically acceptable salt form thereof. Further provided herein is a method for inhibiting growth of leukemia cells in a patient, the method comprising administering to the patient a therapeutically effective amount of a compound of formula (I) or a pharmaceutically acceptable salt form thereof.

In some embodiments, R¹, R², R³, R⁴, R⁵, R⁶, R⁷, and R⁸ are independently a C₁₋₆ alkyl. For example, R¹, R², R³, R⁴, R⁵, R⁶, R⁷, and R⁸ can be CH₃. In some embodiments, R⁹ and R¹⁰ are hydrogen

A non-limiting example of a compound of formula (I) is:

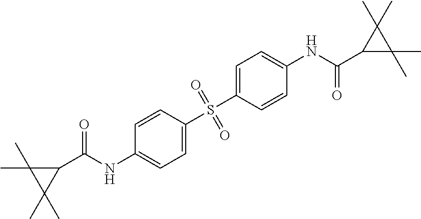

or a pharmaceutically acceptable salt form thereof.

In some embodiments, the patient is in remission. In some embodiments, the leukemia is selected from the group consisting of: chronic eosinophilic leukemia (CEL), chronic neutrophilic leukemia (CNL), chronic myelogenous leukemia (CML), chronic myelomonocytic leukemia (CMML), hairy cell leukemia (HCL), and chronic lymphocytic leukemia (CLL); acute leukemias include acute myelogenous leukemia (AML) and acute lymphocytic leukemia (ALL).

In some embodiments, the leukemia cells are leukemia stem cells.

Further provided herein is a pharmaceutical composition comprising a compound of formula (I), or a pharmaceutically acceptable salt form thereof, and a pharmaceutically acceptable carrier.

This disclosure also provides a method for treating leukemia in a patient, the method comprising administering to the patient a therapeutically effective amount of a compound of formula (II):

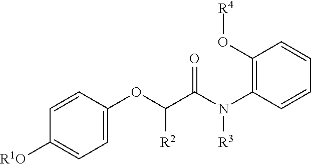

or a pharmaceutically acceptable salt form thereof,

wherein:
R¹, R³, and R⁴ are independently selected from the group consisting of: hydrogen and C₁₋₆ alkyl; and

R² is selected from the group consisting of: hydrogen, C₁₋₆ alkyl, C₁₋₆ alkenyl, and C₁₋₆ alkynyl.

In addition, a method for reducing the number of leukemia cells in a patient is provided, the method comprising administering to the patient a therapeutically effective amount of a compound of formula (II) or a pharmaceutically acceptable salt form thereof. Also provided herein is a method for inhibiting growth of leukemia cells in a patient, the method comprising administering to the patient a therapeutically effective amount of a compound of formula (II) or a pharmaceutically acceptable salt form thereof.

In some embodiments, R¹ and R⁴ are independently a C₁₋₆ alkyl. In some embodiments, R² is a C₁₋₆ alkyl. In some embodiments, R³ is hydrogen.

A non-limiting example of a compound of formula (II) is:

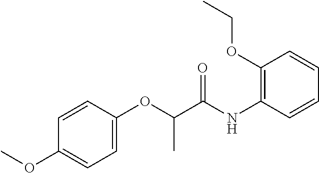 [SMILES: CCOc1ccccc1NC(=O)C(C)Oc1ccc(OC)cc1]

or a pharmaceutically acceptable salt form thereof.

In some embodiments, the patient is in remission. In some embodiments, the leukemia is selected from the group consisting of: chronic eosinophilic leukemia (CEL), chronic neutrophilic leukemia (CNL), chronic myelogenous leukemia (CML), chronic myelomonocytic leukemia (CMML), hairy cell leukemia (HCL), and chronic lymphocytic leukemia (CLL); acute leukemias include acute myelogenous leukemia (AML) and acute lymphocytic leukemia (ALL).

In some embodiments, the leukemia cells are leukemia stem cells.

Further provided herein is a pharmaceutical composition comprising a compound of formula (II) or a pharmaceutically acceptable salt form thereof, and a pharmaceutically acceptable carrier.

This disclosure also provides a method for treating leukemia in a patient, the method comprising administering to the patient a therapeutically effective amount of a compound of formula (III):

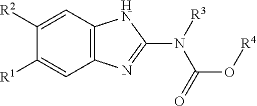

or a pharmaceutically acceptable salt form thereof,

wherein:
R¹ and R² are independently selected from the group consisting of hydrogen, C₁₋₆ alkyl, C₁₋₆ alkenyl, C₁₋₆ alkynyl, OR⁵, C(O)R⁵, SR⁶, S(O)₂R⁵, carbocyclyl, heterocyclyl, aryl, and heteroaryl;
R³ and R⁴ are independently selected from the group consisting of: hydrogen and C₁₋₆ alkyl; and
each R⁵ is independently selected from the group consisting of: hydrogen, C₁₋₆ alkyl, C₁₋₆ alkenyl, C₁₋₆ alkynyl, carbocyclyl, heterocyclyl, aryl, and heteroaryl.

Also provided herein is a method for reducing the number of leukemia cells in a patient, the method comprising administering to the patient a therapeutically effective amount of a compound of formula (III) or a pharmaceutically acceptable salt form thereof. Further provided herein is a method for inhibiting growth of leukemia cells in a patient, the method comprising administering to the patient a therapeutically effective amount of a compound of formula (III) or a pharmaceutically acceptable salt form thereof.

In some embodiments, one of R¹ and R² is hydrogen. In some embodiments, R⁴ is a C₁₋₆ alkyl. For example, R⁴ is CH₃. In some embodiments, R³ is hydrogen.

Non-limiting examples of a compound of formula (III) includes:

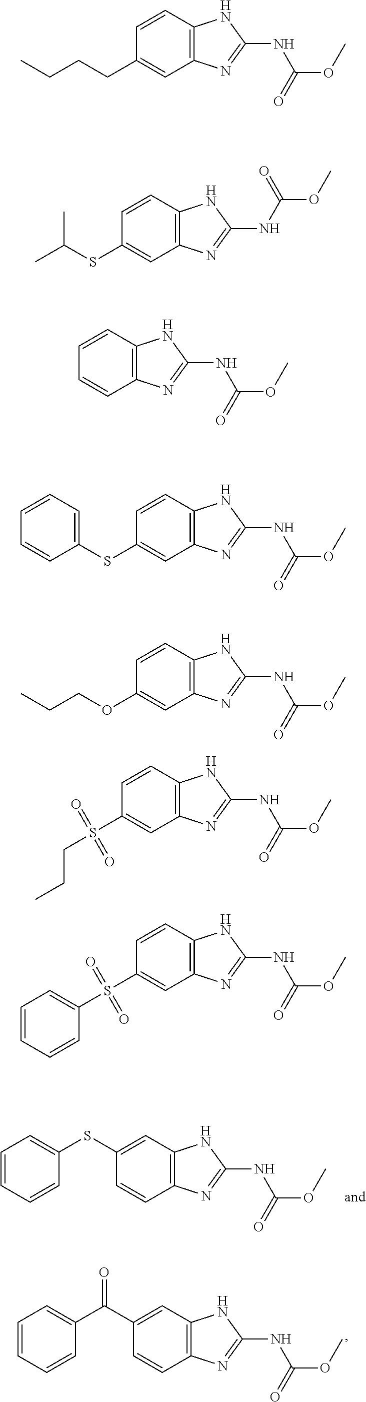

or a pharmaceutically acceptable salt form thereof.

In some embodiments, the patient is in remission. In some embodiments, the leukemia is selected from the group consisting of: chronic eosinophilic leukemia (CEL), chronic neutrophilic leukemia (CNL), chronic myelogenous leukemia (CML), chronic myelomonocytic leukemia (CMML), hairy cell leukemia (HCL), and chronic lymphocytic leukemia (CLL); acute leukemias include acute myelogenous leukemia (AML) and acute lymphocytic leukemia (ALL).

In some embodiments, the leukemia cells are leukemia stem cells.

This disclosure also provides a method for treating leukemia in a patient, the method comprising administering to the patient a therapeutically effective amount of a compound of formula (IV):

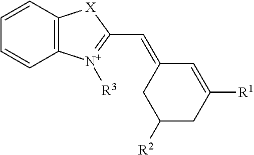

or a pharmaceutically acceptable salt form thereof,

wherein:
R¹ and R² are independently selected from the group consisting of: hydrogen, C₁₋₆ alkyl, C₁₋₆ alkenyl, and C₁₋₆ alkynyl; and
R³ is selected from the group consisting of: hydrogen and C₁₋₆ alkyl.
   Also provided herein is a method for reducing the number of leukemia cells in a patient, the method comprising administering to the patient a therapeutically effective amount of a compound of formula (IV) or a pharmaceutically acceptable salt form thereof. Further provided herein is a method for inhibiting growth of leukemia cells in a patient, the method comprising administering to the patient a therapeutically effective amount of a compound of formula (IV) or a pharmaceutically acceptable salt form thereof.

In some embodiments, R¹ and R² are independently a C₁₋₆ alkyl. For example, R¹ and R² are CH₃. In some embodiments, R³ is selected from the group consisting of hydrogen and C₁₋₆ alkyl. In some embodiments, R⁴ is a C₁₋₆ alkyl. For example, R⁴ is CH₂CH₃.

Non-limiting examples of a compound of formula (IV) includes:

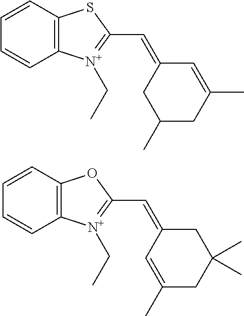 [SMILES: CC[n+]1c(/C=C2/C=C(C)CC(C)C2)sc2ccccc21 ; CC[n+]1c(/C=C2\C=C(C)CC(C)(C)C2)oc2ccccc21]

or a pharmaceutically acceptable salt form thereof.

In some embodiments, the patient is in remission. In some embodiments, the leukemia is selected from the group consisting of: chronic eosinophilic leukemia (CEL), chronic neutrophilic leukemia (CNL), chronic myelogenous leukemia (CML), chronic myelomonocytic leukemia (CMML), hairy cell leukemia (HCL), and chronic lymphocytic leukemia (CLL); acute leukemias include acute myelogenous leukemia (AML) and acute lymphocytic leukemia (ALL).

In some embodiments, the leukemia cells are leukemia stem cells.

Further provided herein is a pharmaceutical composition comprising a compound of formula (IV) or a pharmaceutically acceptable salt form thereof, and a pharmaceutically acceptable carrier.

This disclosure also provides a method for treating leukemia in a patient, the method comprising administering to the patient a therapeutically effective amount of a compound of formula (V):

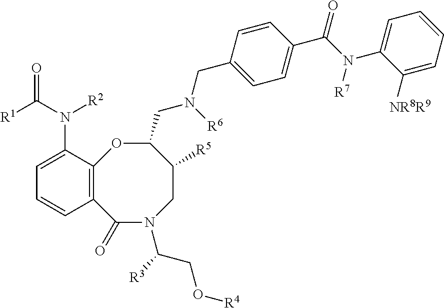

or a pharmaceutically acceptable salt form thereof,

wherein:
R¹ is selected from the group consisting of hydrogen, C₁₋₆ alkyl, C₁₋₆ alkenyl, C₁₋₆ alkynyl, NR¹⁰R¹¹, carbocyclyl, heterocyclyl, aryl, and heteroaryl;
R³ and R⁵ are independently selected from the group consisting of: hydrogen, C₁₋₆ alkyl, C₁₋₆ alkenyl, and C₁₋₆ alkynyl;
R², R⁴, R⁶, R⁷, R⁸, and R⁹ are independently selected from the group consisting of: hydrogen and C₁₋₆ alkyl; and
R¹⁰ and R¹¹ are independently selected from the group consisting of: hydrogen, C₁₋₆ alkyl, C₁₋₆ alkenyl, C₁₋₆ alkynyl, carbocyclyl, heterocyclyl, aryl, and heteroaryl.
   Also provided is a method for reducing the number of leukemia cells in a patient, the method comprising administering to the patient a therapeutically effective amount of a compound of formula (V) or a pharmaceutically acceptable salt form thereof. Further provided herein is a method for inhibiting growth of leukemia cells in a patient, the method comprising administering to the patient a therapeutically effective amount of a compound of formula (V) or a pharmaceutically acceptable salt form thereof.

In some embodiments, R¹ is selected from the group consisting of NR¹⁰R¹¹ and carbocyclyl. In some embodiments, R¹⁰ is hydrogen and R¹¹ is an aryl. In some embodiments, R³ and R⁵ are independently a C₁₋₆ alkyl. In some embodiments, R², R⁴, R⁷, R⁸, and R⁹ are hydrogen. In some embodiments, R⁶ is a C₁₋₆ alkyl.

Non-limiting examples of a compound of formula (V) includes:

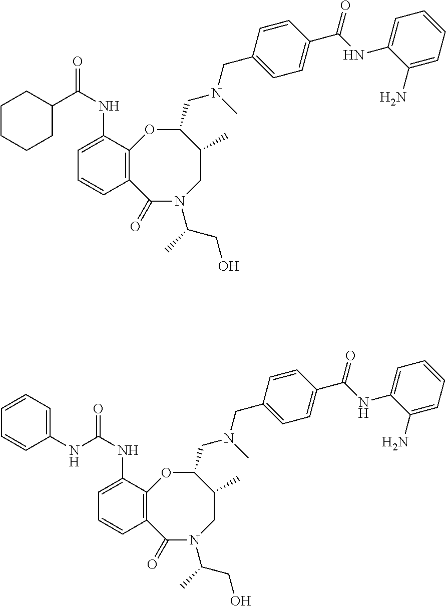 [SMILES: C[C@@H]1CN([C@@H](C)CO)C(=O)c2cccc(NC(=O)C3CCCCC3)c2O[C@@H]1CN(C)Cc1ccc(C(=O)Nc2ccccc2N)cc1 ; C[C@@H]1CN([C@@H](C)CO)C(=O)c2cccc(NC(=O)Nc3ccccc3)c2O[C@@H]1CN(C)Cc1ccc(C(=O)Nc2ccccc2N)cc1]

or a pharmaceutically acceptable salt form thereof.

In some embodiments, the patient is in remission. In some embodiments, the leukemia is selected from the group consisting of: chronic eosinophilic leukemia (CEL), chronic neutrophilic leukemia (CNL), chronic myelogenous leukemia (CML), chronic myelomonocytic leukemia (CMML), hairy cell leukemia (HCL), and chronic lymphocytic leukemia (CLL); acute leukemias include acute myelogenous leukemia (AML) and acute lymphocytic leukemia (ALL).

In some embodiments, the leukemia cells are leukemia stem cells.

Further provided herein is a pharmaceutical composition comprising a compound of formula (V) or a pharmaceutically acceptable salt form thereof, and a pharmaceutically acceptable carrier.

This disclosure also provides a method for treating leukemia in a patient, the method comprising administering to the patient a therapeutically effective amount of a compound of formula (VI):

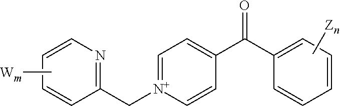

or a pharmaceutically acceptable salt form thereof,

wherein:
W and Z are independently selected from the group consisting of: halogen, OR¹, NR¹R², CN, NO₂, C₁₋₆ alkyl, C₁₋₆ alkenyl, and C₁₋₆ alkynyl;
R¹ and R² are independently selected from the group consisting of: hydrogen and C₁₋₆ alkyl;
m is an integer from 0 to 4; and
n is an integer from 0 to 5.
   Also provided is a method for reducing the number of leukemia cells in a patient, the method comprising administering to the patient a therapeutically effective amount of a compound of formula (VI) or a pharmaceutically acceptable salt form thereof. Further provided is a method for inhibiting growth of leukemia cells in a patient, the method comprising administering to the patient a therapeutically effective amount of a compound of formula (VI) or a pharmaceutically acceptable salt form thereof.

In some embodiments, m is 0. In some embodiments, n is 0.

A non-limiting example of a compound of formula (VI) is:

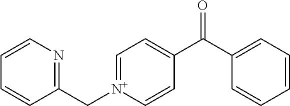 [SMILES: O=C(c1ccccc1)c1cc[n+](Cc2ccccn2)cc1]

or a pharmaceutically acceptable salt form thereof.

In some embodiments, the patient is in remission. In some embodiments, the leukemia is selected from the group consisting of: chronic eosinophilic leukemia (CEL), chronic neutrophilic leukemia (CNL), chronic myelogenous leukemia (CML), chronic myelomonocytic leukemia (CMML), hairy cell leukemia (HCL), and chronic lymphocytic leukemia (CLL); acute leukemias include acute myelogenous leukemia (AML) and acute lymphocytic leukemia (ALL).

In some embodiments, the leukemia cells are leukemia stem cells.

Further provided herein is a pharmaceutical composition comprising a compound of formula (VI) or a pharmaceutically acceptable salt form thereof, and a pharmaceutically acceptable carrier.

This disclosure also provides a method for treating leukemia in a patient, the method comprising administering to the patient a therapeutically effective amount of a compound of formula (VII):

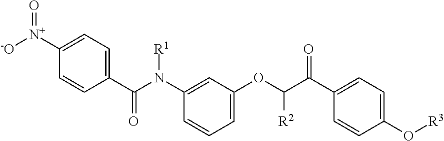

or a pharmaceutically acceptable salt form thereof,

wherein:

R¹ and R³ are independently selected from the group consisting of: hydrogen and C₁₋₆ alkyl; and

R² is selected from the group consisting of: hydrogen, C₁₋₆ alkyl, C₁₋₆ alkenyl, and C₁₋₆ alkynyl.

Also provided is a method for reducing the number of leukemia cells in a patient, the method comprising administering to the patient a therapeutically effective amount of a compound of formula (VII) or a pharmaceutically acceptable salt form thereof. Further provided herein is a method for inhibiting growth of leukemia cells in a patient, the method comprising administering to the patient a therapeutically effective amount of a compound of formula (VII) or a pharmaceutically acceptable salt form thereof.

In some embodiments, R¹ is hydrogen. In some embodiments, R² is a C₁₋₆ alkyl. In some embodiments, R³ is a C₁₋₆ alkyl.

A non-limiting example of a compound of formula (VII) is:

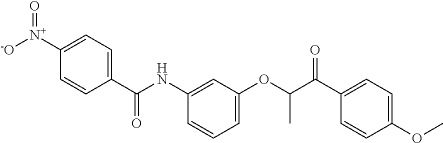

or a pharmaceutically acceptable salt form thereof.

In some embodiments, the patient is in remission. In some embodiments, the leukemia is selected from the group consisting of: chronic eosinophilic leukemia (CEL), chronic neutrophilic leukemia (CNL), chronic myelogenous leukemia (CML), chronic myelomonocytic leukemia (CMML), hairy cell leukemia (HCL), and chronic lymphocytic leukemia (CLL); acute leukemias include acute myelogenous leukemia (AML) and acute lymphocytic leukemia (ALL).

In some embodiments, the leukemia cells are leukemia stem cells.

Further provided herein is a pharmaceutical composition comprising a compound of formula (VII) or a pharmaceutically acceptable salt form thereof, and a pharmaceutically acceptable carrier.

This disclosure also provides a method for treating leukemia in a patient, the method comprising administering to the patient a therapeutically effective amount of a compound of formula (VIII):

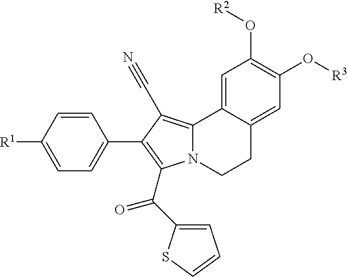

or a pharmaceutically acceptable salt form thereof,

wherein:
R¹ is selected from the group consisting of: hydrogen, C₁₋₆ alkyl, C₁₋₆ alkenyl, and C₁₋₆ alkynyl; and
R² and R³ are independently selected from the group consisting of: hydrogen and C₁₋₆ alkyl.

Also provided herein is a method for reducing the number of leukemia cells in a patient, the method comprising administering to the patient a therapeutically effective amount of a compound of formula (VIII) or a pharmaceutically acceptable salt form thereof. Further provided herein is a method for inhibiting growth of leukemia cells in a patient, the method comprising administering to the patient a therapeutically effective amount of a compound of formula (VIII) or a pharmaceutically acceptable salt form thereof.

In some embodiments, R¹ is a C₁₋₆ alkyl. In some embodiments, R² and R³ are independently a C₁₋₆ alkyl. For example, R² and R³ are CH₃.

A non-limiting example of a compound of formula (VIII) is:

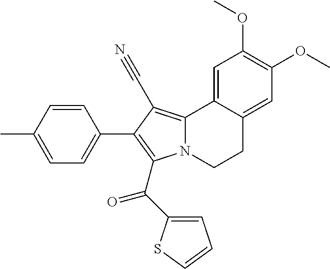

or a pharmaceutically acceptable salt form thereof.

In some embodiments, the patient is in remission. In some embodiments, the leukemia is selected from the group consisting of: chronic eosinophilic leukemia (CEL), chronic neutrophilic leukemia (CNL), chronic myelogenous leukemia (CML), chronic myelomonocytic leukemia (CMML), hairy cell leukemia (HCL), and chronic lymphocytic leukemia (CLL); acute leukemias include acute myelogenous leukemia (AML) and acute lymphocytic leukemia (ALL).

In some embodiments, the leukemia cells are leukemia stem cells.

Further provided herein is a pharmaceutical composition comprising a compound of formula (VIII) or a pharmaceutically acceptable salt form thereof, and a pharmaceutically acceptable excipient.

In any of the methods provided above, the method can further comprise administering at least one treatment for leukemia to the patient. For example, the at least one other treatment is selected from the group consisting of chemotherapy and irradiation. In some embodiments, chemotherapy comprises administration of an agent selected from the group consisting of abarelix, aldesleukin, alemtuzumab, alitretinoin, allopurinol, altretamine, anastrozole, arsenic trioxide, asparaginase, azacitidine, bevacizumab, bexarotene, bleomycin, bortezombi, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, epirubicin, erlotinib, estramustine, etoposide phosphate, etoposide, exemestane, fentanyl citrate, filgrastim, floxuridine, fludarabine, fluorouracil, fulvestrant, gefitinib, gemcitabine, gemtuzumab ozogamicin, goserelin acetate, histrelin acetate, ibritumomab tiuxetan, idarubicin, ifosfamide, imatinib mesylate, interferon alfa 2a, irinotecan, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, oxaliplatin, paclitaxel, pamidronate, panitumumab, pegaspargase, pegfilgrastim, pemetrexed disodium, pentostatin, pipobroman, plicamycin, procarbazine, quinacrine, rasburicase, rituximab, sorafenib, streptozocin, sunitinib, sunitinib maleate, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, vorinostat, and zoledronate.

In some embodiments, the methods provided herein further comprise administration of an additional leukemia-stem cell therapy. For example, CD44 agonism, AMD-3100, parthenolide, celastrol, piperlongumine, and 2-methoxy estradiol.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-1J. A High Throughput Approach for Probing Primary

Leukemia Stem Cells Within a Stromal Niche

(1A) LSC-enriched leukemia cells (medium gray, some of which are indicated by white arrows) generate cobblestoned morphologies when plated on bone marrow stroma (primary MSCs, light grey). The coculture image is shown at 6 days post leukemia cell plating.

(1B) LSC-enriched leukemia cells (c-kit^{hi}) form clusters of cobblestoning cells (arrow) on OP9 stroma with much greater efficiency than the non-stem population (c-kit^{lo}).

(1C) An example of the “nearest neighbors” metric, one of 50 computational rules used for the automated quantification of cobblestoned cells, is depicted here. A raw image of dsRed⁺ leukemia cells (left) is converted to a heatmap (middle) denoting number of adjacent cells (brighter areas=more), which together with 49 other metrics identifies cells (right) that are part of a cobblestone (lighter grey=cobblestoned).

(1D) The results of the primary screen of 14,700 compounds (medium grey) added at 5 μM, plus positive (light grey) and negative (DMSO—dark grey) controls are shown for each replicate. Hits were defined as compounds that reduce the cobblestoned area by greater than 3 standard deviations from the DMSO controls in both replicates.

(1E) Hits from the primary screen were retested at 8-point dose on OP9 stroma and 4-point dose on MSCs. The retest rate is shown. A positive retest was defined as an IC₅₀≦5 μM.

(1F) Stromal toxicity filtering in which the cytotoxic effects of 196 prioritized hits were tested at 8 concentrations on OP9 and primary MSC stromal cells grown in isolation. Compounds showing stromal toxicity on both types of stroma at or below 10 μM were excluded from further study.

(1G) A schematic of the filtering steps employed to define robust and leukemia-selective compounds. The number of compounds at each step is shown in blue.

(1H) Growth of primary leukemia cells in coculture (quantified as total viable cells) is enhanced by addition of media that had been conditioned on stromal monolayers for 3 days. The physical presence of the stromal monolayer provides additional support.

(1I) As few as 100 leukemia cells that had been cocultured ex vivo for 4 weeks can initiate leukemia in recipient mice.

(1J) A schematic of the niche-based small molecule primary screen designed and employed to identify leukemia-selective therapeutics at high throughput scale.

FIGS. 2A-2G. Secondary Screening Identifies Compounds with Distinct Activity Profiles

(2A) In the primary screen, a number of compounds that inhibited leukemia cells caused changes in stromal cell morphology, highlighting the possibility of non-cell-autonomous mechanisms.

(2B) A stromal pretreatment secondary screen identified compounds that antagonize leukemic cobblestoning by modifying the niche. This protocol is an adaptation of the primary screening protocol.

(2C) Troglitazone, a PPAR-γ agonist that induces adipocytic change in the stroma, inhibited leukemia cobblestoning in the stromal pretreatment screen but did not display activity against AML cell lines.

(2D) Comparison of the average IC50 across the cell lines to the IC50 on the primary leukemia cells in coculture identifies compounds that were more than 10-fold more potent on primary cells (bottom right), and compounds that were 10-fold more potent on cell lines (top left).

(2E) The effects of AMD-3100, a known leukemia-selective compound, in the coculture assay. LSC-enriched leukemia cells (leukemic) or normal hematopoietic stem and progenitor cells (normal) were cultured under identical conditions on primary MSC stroma, and the two curves were overlaid as shown.

(2F) Parbendazole and methiazole show leukemia-selective activity in coculture.

(2G) Parbendazole and methiazole also display strong activity against all 6 human AML cell lines tested.

FIG. 3A-3G. A Novel Small Molecule, BRD7116, Selectively Targets Leukemia Cells by Both Cell-Autonomous and Non-Cell-Autonomous Mechanisms

(3A) The chemical structure of the bis-arylsulfone hit, BRD7116, is shown.

(3B) Addition of the compound to cocultures on primary MSC stroma resulted in selective loss of the LSC-enriched leukemia cells (leukemic) compared to normal HSPCs (normal). The data from these separate experiments is shown overlaid.

(3C) In the stromal pretreatment secondary screen, addition of BRD7116 to OP9 stromal cells for three days prior to LSC plating resulted in decreased leukemic cobblestoning, highlighting a non-cell-autonomous mechanism of activity. The original coculture retest data is also shown, overlaid.

(3D) When LSCs and HPSCs were plated together on primary MSC stroma pre-treated with BRD7116, then rinsed, selective inhibition of the leukemia cells is observed.

(3E) BRD7116 induces an AML differentiation program in primary leukemia cells. Compared to DMSO control, gene expression changes present at 6 hours of BRD7116 treatment are significantly enriched by GSEA for the AML differentiation signature seen with the addition of all-trans retinoic acid (ATRA) to ATRA-sensitive human AML cells.

(3F) BRD7116 displayed varying activity against AML cell lines with maximal doses only achieving inhibition to 50% of the positive control.

(3G) An induction of apoptosis was quantified by Annexin V staining after 22 hours of treatment with BRD7116.

FIG. 4A-4H. Coculture Screening and In vivo RNAi Secondary Screening Reveal Selective Sensitivity to HMG-CoA Reductase Inhibition in Leukemia Stem Cells

(4A) The chemical structure of coculture screening hit lovastatin is shown.

(4B) lovastatin displays leukemia-selective activity (leukemic) compared to HSPCs (normal) when grown in coculture with MSCs (top), and only weak activity against human AML cell lines (bottom).

(4C) Addition of lovastatin (black bars) for 24 hours at either day 1 or 4 post-LSC plating results in near-complete loss of leukemia cells at the end of the coculture assay while having no effect on stromal viability (CellTiter-Glo) compared to DMSO control (gray bars).

(4D) The addition of mevalonolactone (mevalon) rescues the anti-leukemia effect of lovastatin in coculture, demonstrating an HMGCR inhibition mechanism of lovastatin activity.

(4E) An in vivo shRNA pooled screen targeting the mevalonate metabolism pathway validates an Hmgcr dependency in leukemia stem cells, and links the coculture platform to the true bone marrow niche. The most strongly depleted shRNAs (normalized to control shRNAs) are marked: *, 20-fold; and +, 10-fold.

(4F) An induction of apoptosis was quantified by Annexin V staining after 22 hours of treatment with lovastatin.

(4G) The effects of various chemical inhibitors of farnesyltransferase and geranylgeranyl transferase are shown for LSC-enriched leukemia cells cocultured on OP9 stromal cells.

(4H) A farnesyltransferase inhibitor, L-744832, independently hit in primary screening, was resourced, then tested against LSCs cocultured on MSC stroma, shown here.

FIGS. 5A-5E. Triple Cocultures and Syngeneic Transplants Further Validate the Selectivity of BRD7116 and Lovastatin

(5A) Representative images of triple cocultures containing both primary LSCs (red) and HSPCs (green) grown on uncolored MSCs. 5 days of exposure to either BRD7116 or lovastatin resulted in selective loss of the leukemia population. The dose response curves for BRD7116 (top graph) and lovastatic acid (bottom graph) across four concentrations in this triple coculture setup are depicted.

(5B, 5C) Kaplan-Meier survival curves of mice transplanted with contents of triple cocultures after 48 hours of treatment with BRD7116 (5B), lovastatin (5C), or DMSO are shown.

(5D, 5E) At 16 weeks, coculture-treated normal HSPCs (CD45.1⁺) display comparable, high levels of bone marrow engraftment across treatment groups in the surviving mice (5D), with similar differentiation patterns evidenced in the bone marrow at the same timepoint (5E).

FIGS. 6A-6F. Effects of BRD7116 and Lovastatin on Primary Human CD34+ Leukemic and Normal Hematopoietic Cells

(6A, 6B) The cobblestone area-forming cell (CAFC) assay was used to determine the effects of BRD7116 (6A) and lovastatin (6B) on human stem cell activity using primary CD34⁺ cells enriched from either normal human cord blood (“Normal”) or 6 different primary AML leukemia patient samples (Lettered A-F). The primary CD34⁺ cells were exposed to small molecules for 18 hours, then rinsed and plated onto supportive MS-5 stromal monolayers. The fraction of replicate platings that contained cobblestones at 5 weeks (2 weeks for FLT3-ITD sample) is shown for each compound relative to DMSO control. BRD7116 and lovastatin were tested in an in vitro progenitor toxicity assay using normal primary CD34⁺ cells isolated from the bone marrow (blue), peripheral blood (purple) or cord blood (red) of healthy patients.

(6A-6F) The cells, cultured in suspension, were exposed to compounds for 7 days then analyzed for viability using Alamar Blue staining Whereas therapeutic-range doses of conventional chemotherapies Daunorubicin (6C) and Ara-C (6D) kill progenitors in this assay relative to DMSO control (consistent with observed myelosuppression in patients), BRD7116 (6E) and lovastatin (6F) displayed minimal toxicity.

FIG. 7 is a diagram of an example of a network environment for training a classifier to identify compounds for the treatment of leukemia.

FIG. 8 is a diagram of rules included in a classifier.

FIG. 9 is a block diagram showing examples of components of a network environment for training a classifier to identify compounds for the treatment of leukemia.

FIG. 10 is a flowchart showing an example process for training a classifier to identify compounds for the treatment of leukemia.

FIG. 11 shows an example of a computer device that can be used with the techniques described here.

FIG. 12 illustrates an examination of effects of lovastatin on primary human cells. Effects at ten-fold greater working concentrations than the early estimated IC₅₀ values in murine coculture screen are shown. Both normal (12A) and leukemic (12B) primary CD34+ cells were examined in the CAFC assay as shown.

FIG. 13 shows a first examination of effects of benzimidazole hits on human cells. Effects at ten-fold greater working concentrations than the early estimated IC50 values in murine coculture screen are shown. Both normal (13A) and leukemic (13B) primary CD34+ cells were examined in the CAFC assay as shown.

### DETAILED DESCRIPTION

Increasing evidence indicates that the bone marrow niche, a heterotypic microenvironment known to maintain normal hematopoietic physiology, plays an important role in leukemia development, progression, and therapeutic response (Ishikawa, F., Yoshida, S., Saito, Y., Hijikata, A., Kitamura, H., Tanaka, S., Nakamura, R., Tanaka, T., Tomiyama, H., Saito, N., et al. (2007). Nat Biotechnol 25, 1315-1321.; Raaijmakers, M. H., Mukherjee, S., Guo, S., Zhang, S., Kobayashi, T., Schoonmaker, J. A., Ebert, B. L., Al-Shahrour, F., Hasserjian, R. P., Scadden, E. O., et al. (2010). Nature 464, 852-857.; Wei, J., Wunderlich, M., Fox, C., Alvarez, S., Cigudosa, J. C., Wilhelm, J. S., Zheng, Y., Cancelas, J. A., Gu, Y., Jansen, M., et al. (2008). Cancer Cell 13, 483-495., Funayama, K., Murai, F., Shimane, M., Nomura, H., and Asano, S. (2010). Pharmacology 86, 79-84.; Gehrke, I., Gandhirajan, R. K., Poll-Wolbeck, S. J., Hallek, M., and Kreuzer, K. A. (2011). Mol Med 17, 619-627.; Iwamoto, S., Mihara, K., Downing, J. R., Pui, C. H., and Campana, D. (2007). J Clin Invest 117, 1049-1057.). For example, CD44, VLA-4, and CD47 all appear to mediate non-cell-autonomous interactions, and inhibitors of these signals display activities in mouse models of leukemia (Jin, L., Hope, K. J., Zhai, Q., Smadja-Joffe, F., and Dick, J. E. (2006). Nat Med 12, 1167-1174.; Matsunaga, T., Takemoto, N., Sato, T., Takimoto, R., Tanaka, I., Fujimi, A., Akiyama, T., Kuroda, H., Kawano, Y., Kobune, M., et al. (2003). Nat Med 9, 1158-1165.; Chao, M. P., Alizadeh, A. A., Tang, C., Jan, M., Weissman-Tsukamoto, R., Zhao, F., Park, C. Y., Weissman, I. L., and Majeti, R. (2011). Cancer Res 71, 1374-1384.). Additionally, small molecule inhibitors of the SDF-1-CXCR4 axis have been shown to augment traditional chemotherapies in animal models (Zeng, Z., Shi, Y. X., Samudio, I. J., Wang, R. Y., Ling, X., Frolova, O., Levis, M., Rubin, J. B., Negrin, R. R., Estey, E. H., et al. (2009). Blood 113, 6215-6224.; Nervi, B., Ramirez, P., Rettig, M. P., Uy, G. L., Holt, M. S., Ritchey, J. K., Prior, J. L., Piwnica-Worms, D., Bridger, G., Ley, T. J., et al. (2009). Blood 113, 6206-6214.) and are currently under study in clinical AML, as are Notch inhibitors (Shih Ie, M., and Wang, T. L. (2007). Cancer Res 67, 1879-1882.). Thus, further efforts to elucidate both cell-autonomous and non-cell-autonomous dependencies, particularly in LSCs, may provide important new biological and therapeutic insights in leukemia.

Despite the known importance of LSCs and the microenvironment, traditional drug discovery efforts do not typically incorporate aspects of in vivo pathophysiology, largely due to the technical challenges of capturing such complex biology robustly in scalable format. For example, cell-based small molecule screening generally examines such effects on the viability of cell lines grown in isolation (Caponigro, G., and Sellers, W. R. (2011). Nat Rev Drug Discov 10, 179-187.), without particular appreciation of the richness of both LSC and niche biology. Screening strategies that enable the growth of cancer stem cells, e.g., primary LSC-enriched cellular material in a supportive stromal microenvironment with the examination of a biologically-relevant readout would likely prove beneficial. While progress has been made towards functional in vivo screening on the genetics side (Bric, A., Miething, C., Bialucha, C. U., Scuoppo, C., Zender, L., Krasnitz, A., Xuan, Z., Zuber, J., Wigler, M., Hicks, J., et al. (2009). Cancer Cell 16, 324-335.; Luo, B., Cheung, H. W., Subramanian, A., Sharifnia, T., Okamoto, M., Yang, X., Hinkle, G., Boehm, J. S., Beroukhim, R., Weir, B. A., et al. (2008). Proc Natl Acad Sci U S A 105, 20380-20385.; Mendez-Ferrer, S., Michurina, T. V., Ferraro, F., Mazloom, A. R., Macarthur, B. D., Lira, S. A., Scadden, D. T., Ma'ayan, A., Enikolopov, G. N., and Frenette, P. S. (2010). Nature 466, 829-834.), large scale small molecule screening remains a powerful complementary approach.

Described herein is an experimental paradigm to expansively and systematically probe LSC biology within the context of an ex vivo bone marrow niche using a stem cell-associated readout. This approach was used to identify small molecules that selectively inhibit LSCs by both cell intrinsic and microenvironmental-based effects. Both novel and previously established compounds were identified that kill LSCs while sparing HSPCs, a subset of which would not have been revealed by traditional cell-line based screens. Importantly, these compounds were validated in a series of assays using primary murine and human cells. These findings demonstrate that an incorporation of complex, primary disease biology is feasible in vitro at high throughput scale and provide an innovative framework for defining promising new avenues for therapeutic intervention. In addition, the findings demonstrate that these compounds can be used to treat leukemia, potentially targeting and reducing the number of leukemic stem cells.

Leukemia

Leukemias are heterogeneous neoplastic disorders of white blood cells that can be divided into two classes based on myeloid or lymphoid origin. Leukemias are typically designated as either acute or chronic; acute leukemias are often associated with symptoms including anemia, infection, hemorrhage, or organ compromise/infiltration, including congestive heart failure secondary to severe anemia. Chronic leukemias include chronic eosinophilic leukemia (CEL), chronic neutrophilic leukemia (CNL), chronic myelogenous leukemia (CML), chronic myelomonocytic leukemia (CMML), hairy cell leukemia (HCL), and chronic lymphocytic leukemia (CLL); acute leukemias include acute myelogenous leukemia (AML) and acute lymphocytic leukemia (ALL).

Table A provides further information regarding these types of leukemia.

TABLE A

| Type | Affected Cells | Diagnosis | Treatment |
|---|---|---|---|
| CML | Granulocyte proliferation; | Presence of leuko- | Palliative; Antibiotics; |
| | also erythroid cells and | cytosis is in excess | blood product |
| | megakaryocytes | of 100,000/mm³, | transfusion; |
| | A balanced reciprocal | presence of the Ph | imatinib, |
| | translocation occurs between | chromosome [t(9;22)] | leukopheresis, |
| | the long arms of | or the bcr/abl fusion | busulfan or |
| | chromosomes 9 and 22 | gene | hydroxyurea; blastic |
| | [t(9; 22)(q34; q11.2)] | | phase: vincristine and |
| | in about 90% to 95% of cases | | prednisone; |
| | | | allogeneic bone |
| | | | marrow transplant |
| CLL | Malignant clonal expansion | Presence of lympho- | Palliative; |
| | of lymphocytes (95% are | cytosis of greater | Antibiotics; blood |
| | B lymphocytes, 5% are | than 5,000/mm³ | product transfusion; |
| | T-cell clones) | | chlorambucil, with or |
| | | | without |
| | | | corticosteroids, |
| | | | cyclophosphamide- |
| | | | vincristine- |
| | | | prednisone (CVP), |
| | | | and purine analogues |
| | | | (e.g., fludarabine, |
| | | | cladribine); |
| | | | Rituximab; |
| | | | alemtuzumab; |
| | | | autologous and |
| | | | allogeneic |
| | | | hematopoietic stem |
| | | | cell transplantation |
| CMML | Clonal hematopoietic stem | See, e.g., Vardiman | Antibiotics; blood |
| | cell disorder with dysplasia | et al., “Chronic | product transfusion; |
| | in at least one myeloid | myelomonocytic | growth factors (e.g., |
| | lineage, less than 20% | leukemia.” In Jaffe | granulocyte colony- |
| | blasts in the blood and | et al. (eds), World | stimulating factor, |
| | bone marrow, a persistent | Health Organization | granulocyte- |
| | monocytosis, and no | Classification of | macrophage colony- |
| | evidence of Philadelphia | Tumours. Pathology | stimulating factor, |
| | (Ph) chromosome or the | and Genetics. | erythropoietin),; |
| | bcr/abl fusion gene | Tumours of | amifostine, |
| | | Haematopoietic and | immunosuppressive |
| | | Lymphoid Tissues. | therapy (e.g., |
| | | Lyon, France, IARC | antithymocyte |
| | | Press, 2001, pp 17-31, | globulin, |
| | | 47-52 | cyclosporine); |
| | | | hypomethylating |
| | | | agents (e.g., |
| | | | azacytidine, |
| | | | decitabine), low- |
| | | | intensity |
| | | | chemotherapy (e.g., |
| | | | hydroxyurea), high- |
| | | | intensity |
| | | | chemotherapy (e.g., |
| | | | topotecan), and |
| | | | allogeneic |
| | | | hematopoietic stem |
| | | | cell transplantation |
| CNL | Sustained, mature neutro- | See, e.g., Vardiman | Hydroxyurea, |
| | philic leukocytosis | et al., supra | busulfan, 6- |
| | | | thioguanine, and |
| | | | interferon |
| CEL | cLonal proliferation of | See, e.g., Vardiman | Imatinib, |
| | eosinophilic precursors | et al., supra | hydroxyurea, |
| | with increased blasts | | busulfan, 6- |
| | | | thioguanine, and |
| | | | interferon |
| HCL | Malignancy of small B | Hairy cells detected | Cladribine; |
| | lymphoid cells that display | on morphologic | pentostatin; |
| | surface cytoplasmic “hairy” | examination of | splenectomy; |
| | projections | peripheral blood | interferon alfa; |
| | | | rituximab; anti-CD22 |
| | | | recombinant |
| | | | immunotoxin BL22 |
| AML | Malignant clonal expansion | Greater than 20% | Daunorubicin; |
| | of bone marrow hemato- | leukemic blasts in | cytarabine; |
| | poietic precursor cells | the bone marrow, | idarubicin; |
| | | confirmed commit- | mitoxantrone |
| | | ment to myeloid | thioguanine; arsenic |
| | | lineage | trioxide; |
| | | | gemtuzumab |
| | | | ozogamicin; bone |
| | | | marrow transplant |
| ALL | Malignant clonal expansion | Greater than 20% | vincristine, |
| | of bone marrow lympho- | leukemic blasts in | prednisone, and L- |
| | poietic precursor cells | the bone marrow, | asparaginase |
| | | confirmed commit- | |
| | | ment to lymphoid | |
| | | lineage | |

Tests for diagnosing the presence of a leukemia in a subject include the complete blood count (CBC); bone marrow aspiration; immunophenotyping (particularly for ALL to determine B or T cell origin); histochemical stains (e.g., for myeloperoxidase, nonspecific esterase, or nuclear DNA polymerizing enzyme terminal deoxynucleotidyl transferase (TdT)); chromosomal analysis; fluorescein angiography; and optical coherence tomography (OCT). See, e.g., Vardiman et al., “Chronic myelomonocytic leukemia.” In Jaffe et al. (eds), World Health Organization Classification of Tumours. Pathology and Genetics. Tumours of Haematopoietic and Lymphoid Tissues. Lyon, France, IARC Press, 2001, pp 17-31, 47-52; “National Cancer Institute-sponsored Working Group guidelines for chronic lymphocytic leukemia: Revised guidelines for diagnosis and treatment.” Blood. 87: 1996; 4990-4997; and “The World Health Organization (WHO) classification of the myeloid neoplasms.” Blood. 100: 2002; 2292-2302.

In some embodiments, the methods described herein include the administration of post-remission therapy with an agent, e.g., an agent described herein, that targets LSCs identified by a method described herein. Subjects who are in remission can be identified by methods known in the art, e.g., a return to normal or near-normal levels of a cell or cell-type that was previously abnormal.

Methods of Identifying Compounds for the Treatment of Leukemia

Provided herein are semi-automated, computer-aided methods of identifying compounds for the treatment of leukemia. The methods are particularly useful for identifying compounds that target leukemia stem cells (LSCs). The methods include screening test compounds, e.g., polypeptides, polynucleotides, inorganic or organic large or small molecule test compounds, to identify agents useful in the treatment of leukemia.

As used herein, “small molecules” refers to small organic or inorganic molecules of molecular weight below about 3,000 Daltons. In general, small molecules useful for the invention have a molecular weight of less than 3,000 Daltons (Da). The small molecules can be, e.g., from at least about 100 Da to about 3,000 Da (e.g., between about 100 to about 3,000 Da, about 100 to about 2500 Da, about 100 to about 2,000 Da, about 100 to about 1,750 Da, about 100 to about 1,500 Da, about 100 to about 1,250 Da, about 100 to about 1,000 Da, about 100 to about 750 Da, about 100 to about 500 Da, about 200 to about 1500, about 500 to about 1000, about 300 to about 1000 Da, or about 100 to about 250 Da).

The test compounds can be, e.g., natural products or members of a combinatorial chemistry library. A set of diverse molecules should be used to cover a variety of functions such as charge, aromaticity, hydrogen bonding, flexibility, size, length of side chain, hydrophobicity, and rigidity. Combinatorial techniques suitable for synthesizing small molecules are known in the art, e.g., as exemplified by Obrecht and Villalgordo, Solid-Supported Combinatorial and Parallel Synthesis of Small-Molecular-Weight Compound Libraries, Pergamon-Elsevier Science Limited (1998), and include those such as the “split and pool” or “parallel” synthesis techniques, solid-phase and solution-phase techniques, and encoding techniques (see, for example, Czarnik, Curr. Opin. Chem. Bio. 1:60-6 (1997)). In addition, a number of small molecule libraries are commercially available. A number of suitable small molecule test compounds are listed in U.S. Pat. No. 6,503,713, incorporated herein by reference in its entirety.

Libraries screened using the methods of the present invention can comprise a variety of types of test compounds. A given library can comprise a set of structurally related or unrelated test compounds. In some embodiments, the test compounds are peptide or peptidomimetic molecules. In some embodiments, the test compounds are nucleic acids.

In some embodiments, the test compounds and libraries thereof can be obtained by systematically altering the structure of a first test compound, e.g., a first test compound that is structurally similar to a known natural binding partner of the target polypeptide, or a first small molecule identified as capable of binding the target polypeptide, e.g., using methods known in the art or the methods described herein, and correlating that structure to a resulting biological activity, e.g., a structure-activity relationship study. As one of skill in the art will appreciate, there are a variety of standard methods for creating such a structure-activity relationship. Thus, in some instances, the work may be largely empirical, and in others, the three-dimensional structure of an endogenous polypeptide or portion thereof can be used as a starting point for the rational design of a small molecule compound or compounds. For example, in one embodiment, a general library of small molecules is screened, e.g., using the methods described herein.

In general, the screening methods include contacting a test compound with a test sample. The test samples used in the screening methods described herein include co-cultures with both cancer cells, e.g., primary cancer cells, e.g., primary hematopoietic cells, e.g., leukemic cells (preferably enriched for LSCs) and/or normal cells (preferably enriched for HSCs and progenitor cells), and stromal (supporting) cells. In some embodiments the test samples include both LSCs and normal cells (e.g., HPSCs) commingled together in a “triple coculture,” which is useful for a side-by side comparison of effects and crosstalk effects. The test samples will typically be present in a multi-well plate or culture dish or other format suitable for high-throughput detection.

The primary hematopoietic cells are preferably enriched for stem and progenitor cells, are preferably mammalian, and can be obtained using methods known in the art. For example, the primary hematopoietic cells can be obtained from the bone marrow of a rodent, e.g., a mouse or rat, or other experimental animal. Alternatively, the primary hematopoietic cells can be human in origin, e.g., obtained from a bone marrow aspiration, e.g., from a subject. In some embodiments, the primary hematopoietic cells can be genetically engineered to express a detectable marker, such as a fluorescent protein (e.g., green fluorescent protein or a variant thereof as known in the art), that allows identification of the primary hematopoietic cells in the test sample such that the formation of cobblestone areas can be detected (in embodiments where no genetic marker is used, detection of a cell surface marker, or brightfield microscopy, can be used). In some embodiments, the methods include enriching the primary hematopoietic cells for stem cells, e.g., by sorting the cells and selecting those with markers known to be associated with stem cells, e.g., c-kit^{hi} or CD34+CD38−. Methods known in the art, e.g., flow cytometry/fluorescence assisted cell sorting can be used to enrich the cells for stem cells.

The stromal cells useful in the test samples can include primary and/or cultured stromal cells. Stromal cells are any non parenchymal cells, also referred to as connective tissue cells, and are typically adherent when bone marrow is grown in culture. They constitute the non-blood forming fraction of bone marrow, and are sometimes referred to also as mesenchymal stromal cells or multipotent mesenchymal stromal cells (Brinchmann, 2008). Such cells have the potential to differentiate into various stromal cell types, such as osteoblasts and adipocytes. Other examples of stromal cell types include endothelial and perivascular cells. Stromal cell lines include 1×N/2b; AC6.21; AFT024; AGM-S3; FLS4.1; FS-1; HAS303; HCB1-SV40; HESS-5; HM1-SV40; HM2-SV40; HYMEQ-5; KM102; L87/4; MRL104.8a; MS-5; OP9; PA6; PK-2; PU-34; S10; S17; S21; Saka; SCL1-24; SC-MSC; SPY3-2; SR-4987; SSL 1; ST-1; ST2; and TBR59 cell lines. In some embodiments, the stromal cells and the primary hematopoietic cells are from the same species. In some embodiments, the stromal cells are also genetically engineered to express a detectable marker that is different from the detectable marker expressed by the primary hematopoietic cells, to allow the differentiation of the two cell types in culture.

In some embodiments, the samples are treated with addition of pre-conditioned media from a stromal cell culture, e.g., as described herein.

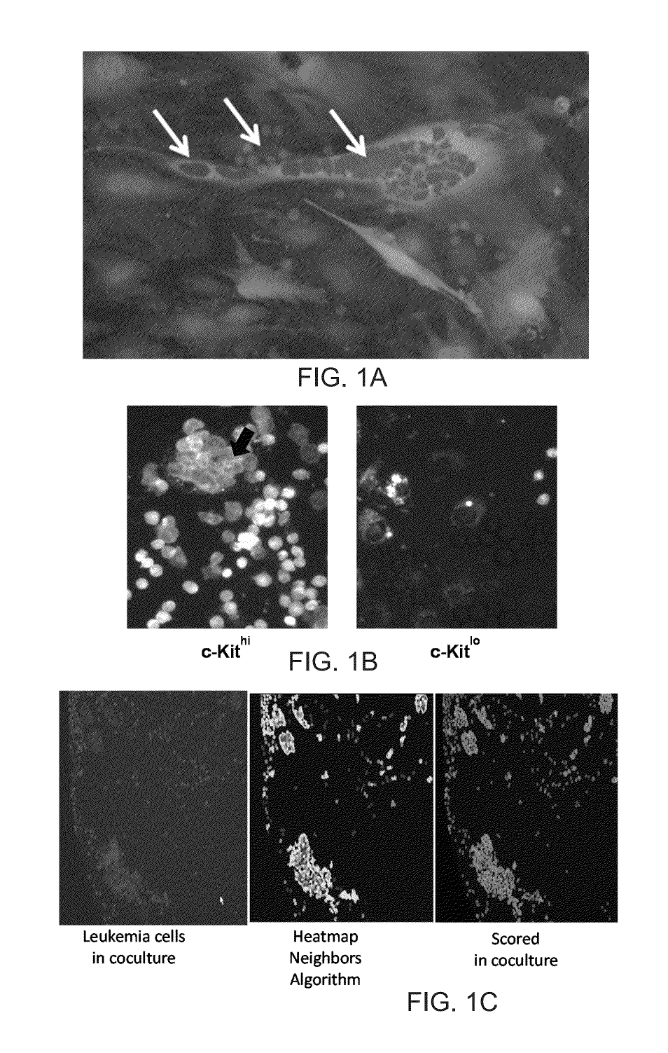

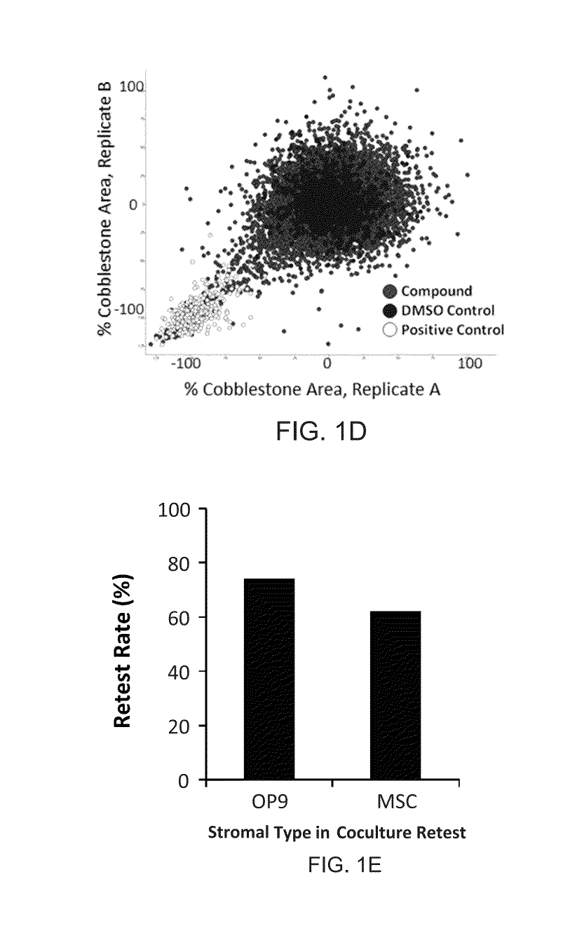

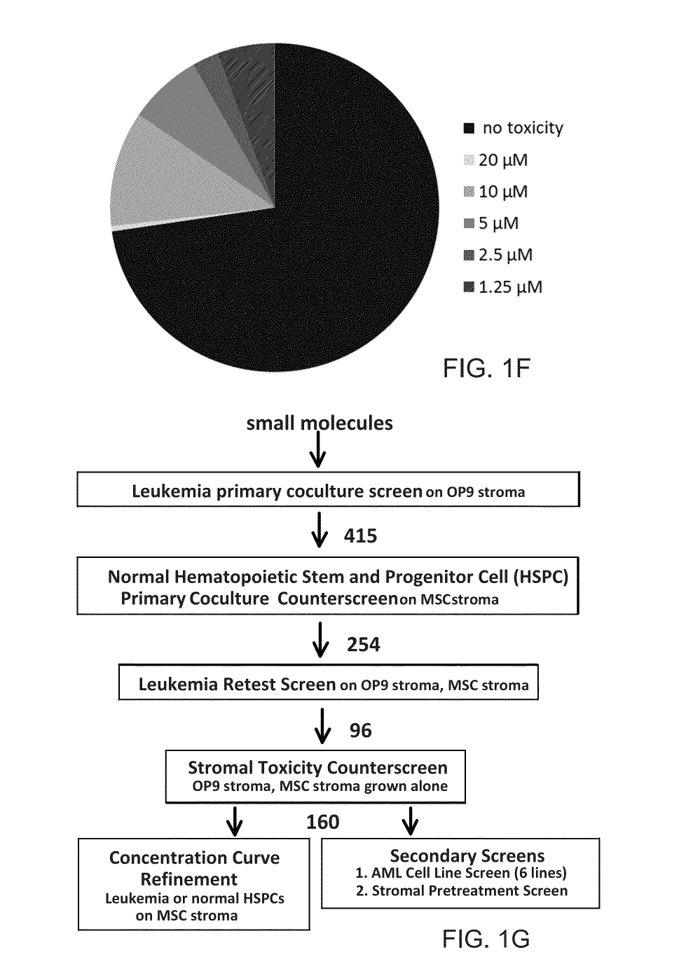

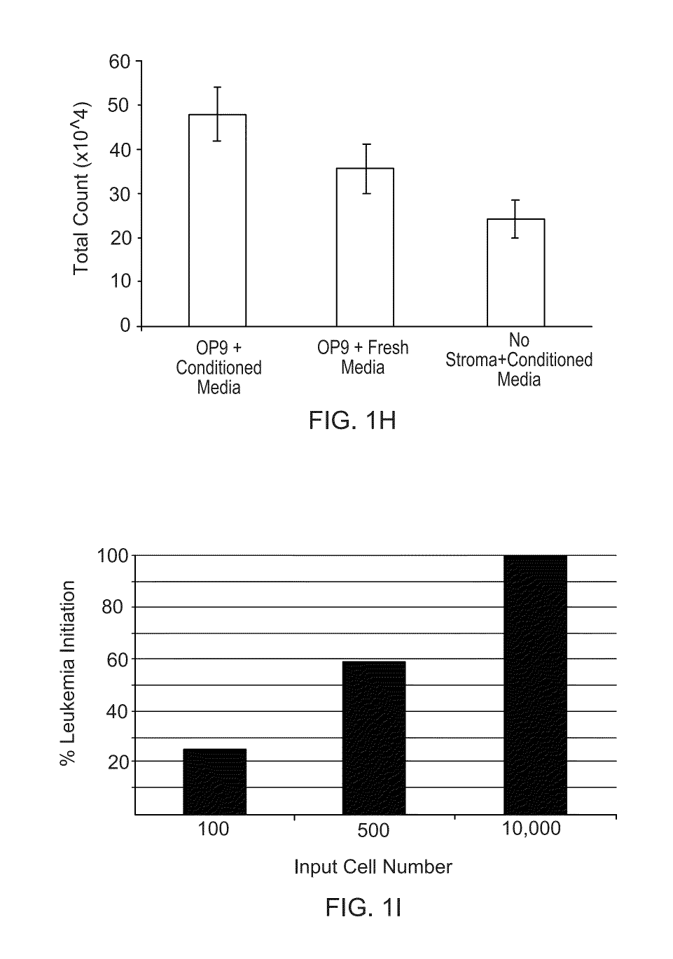

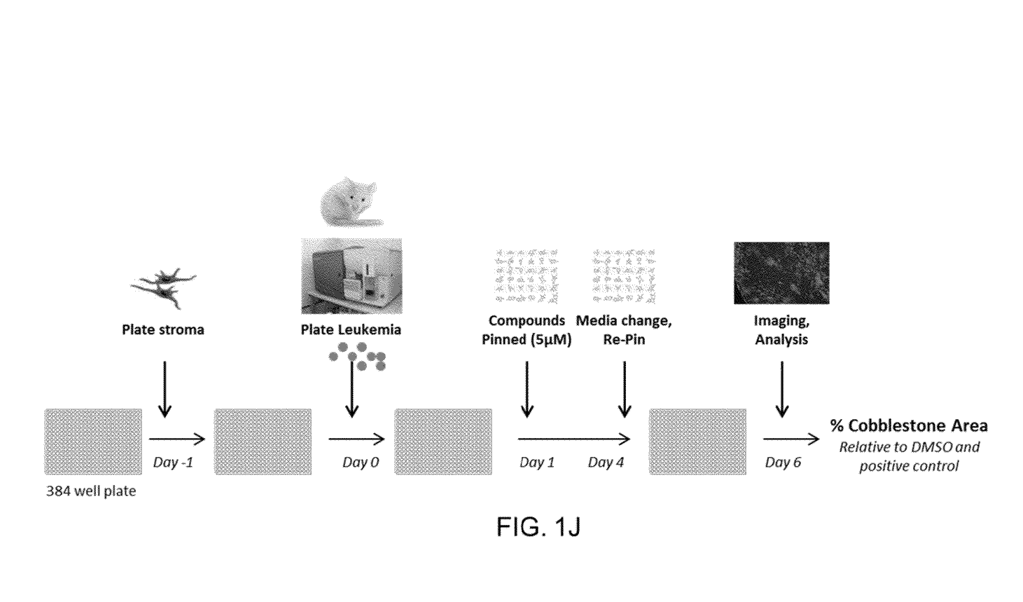

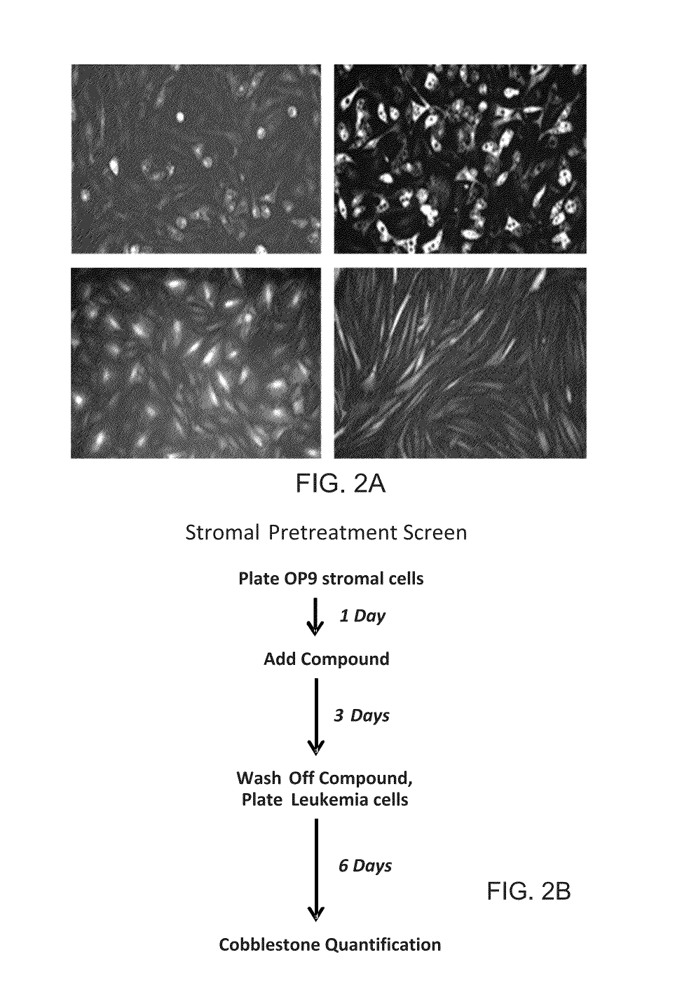

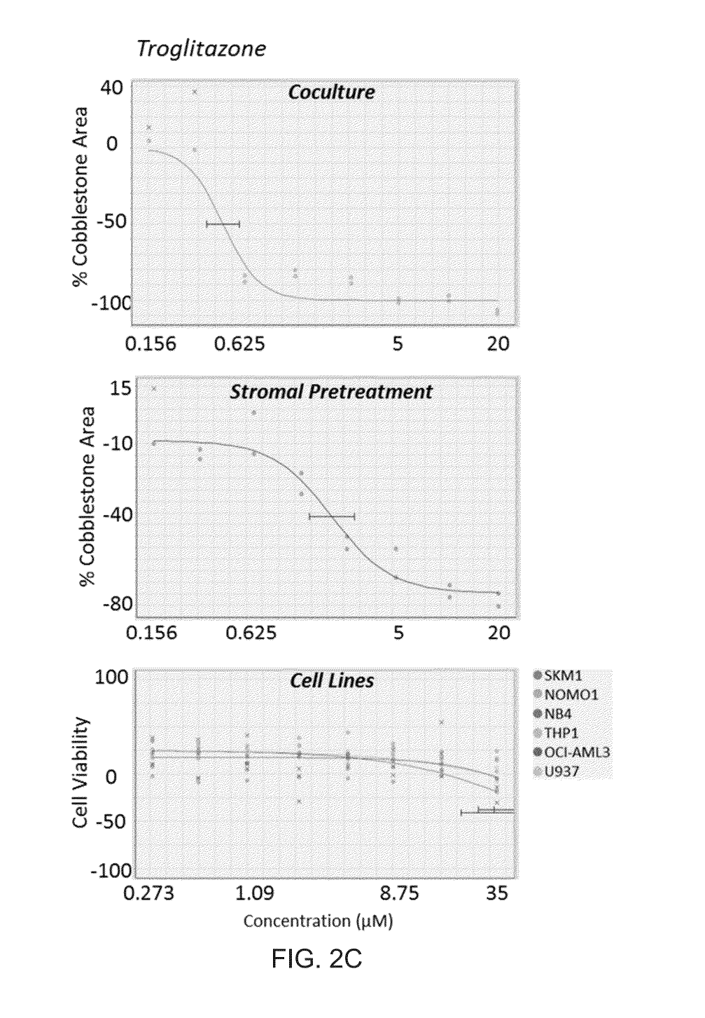

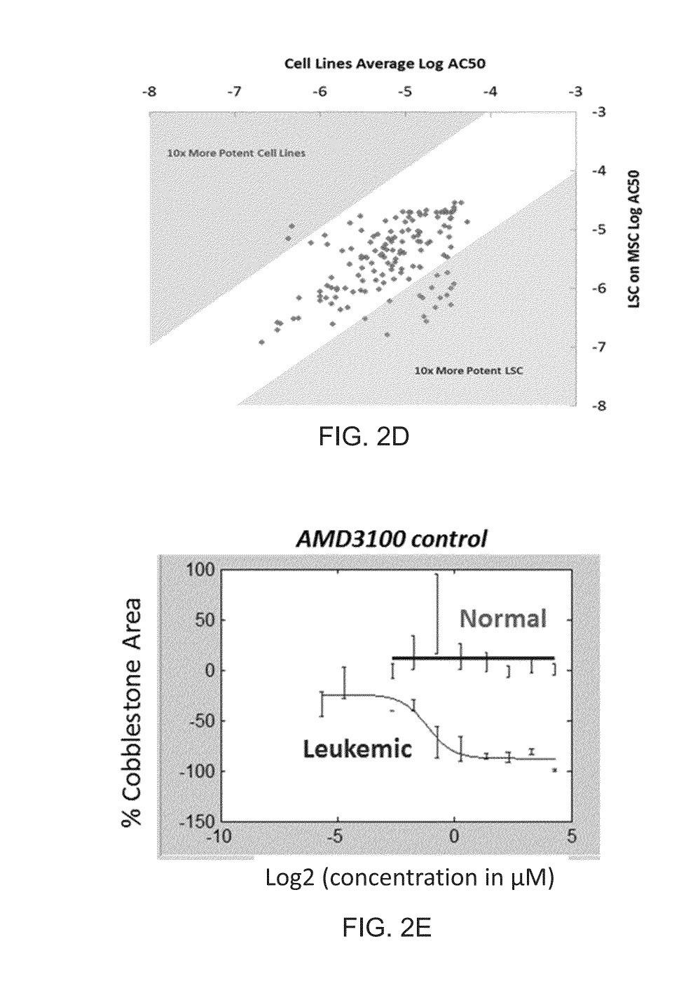

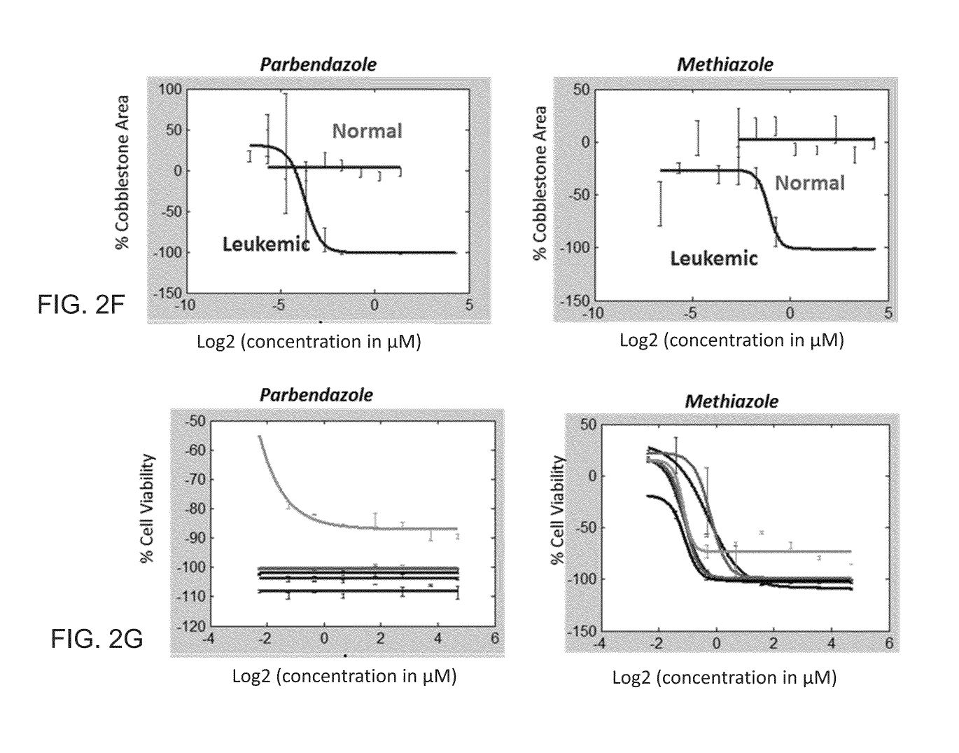

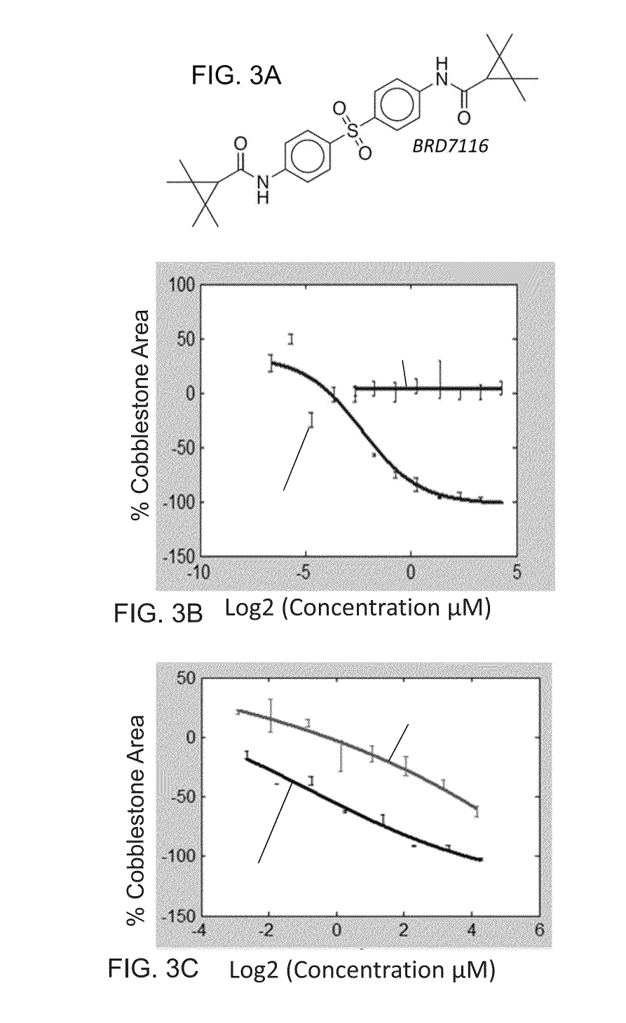

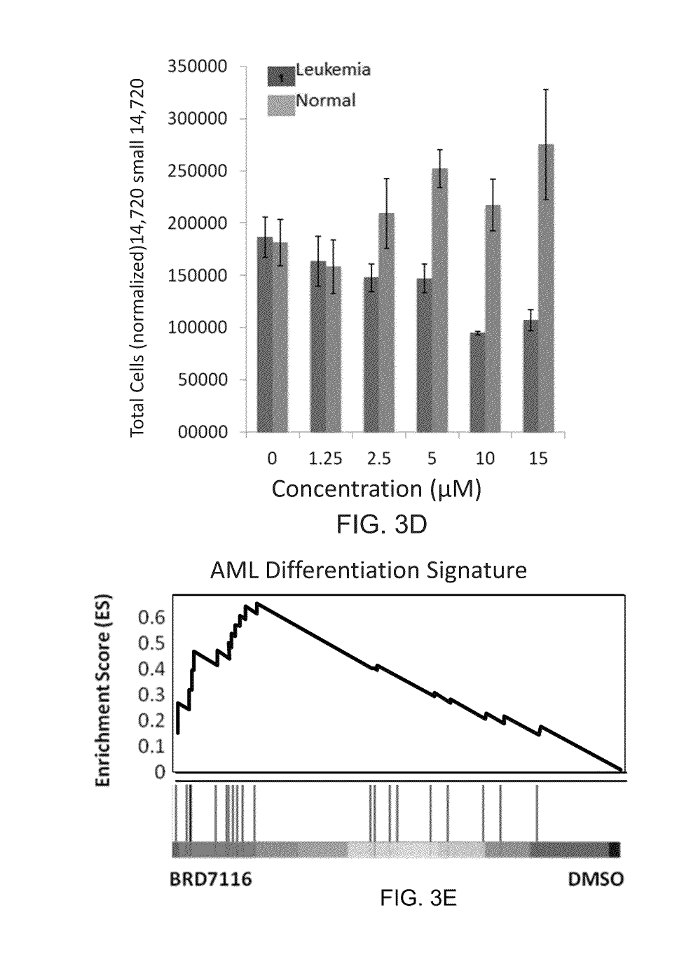

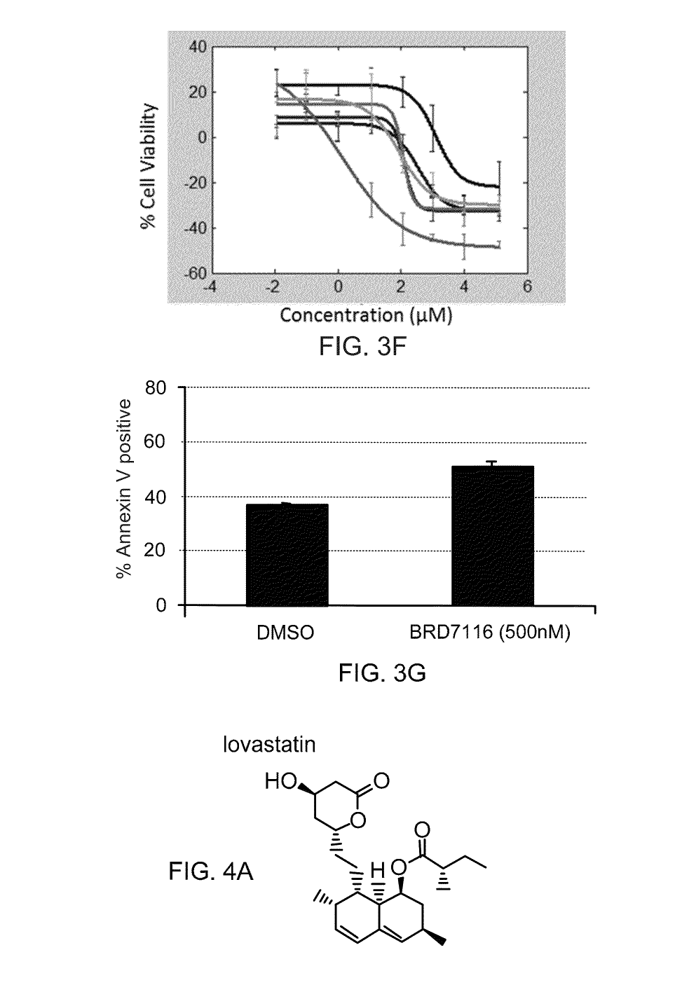

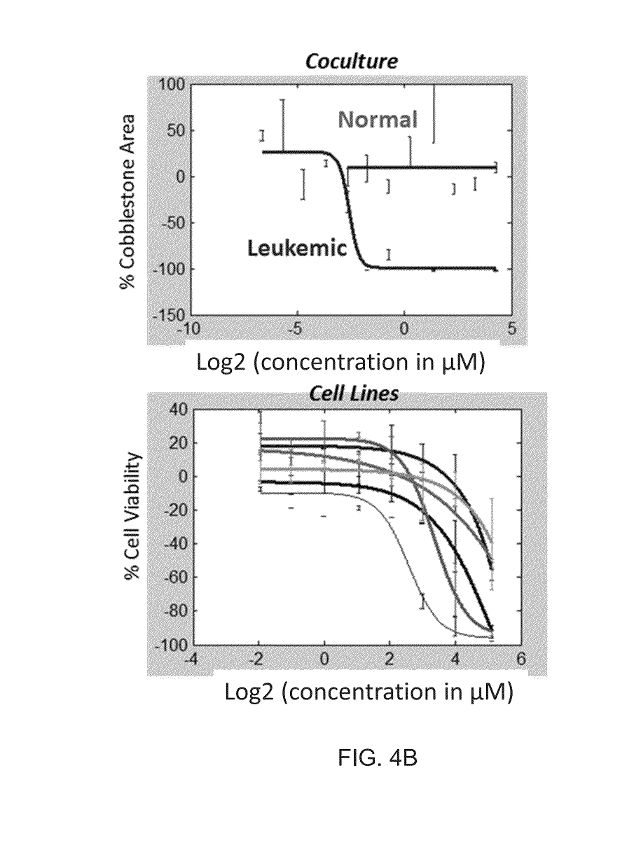

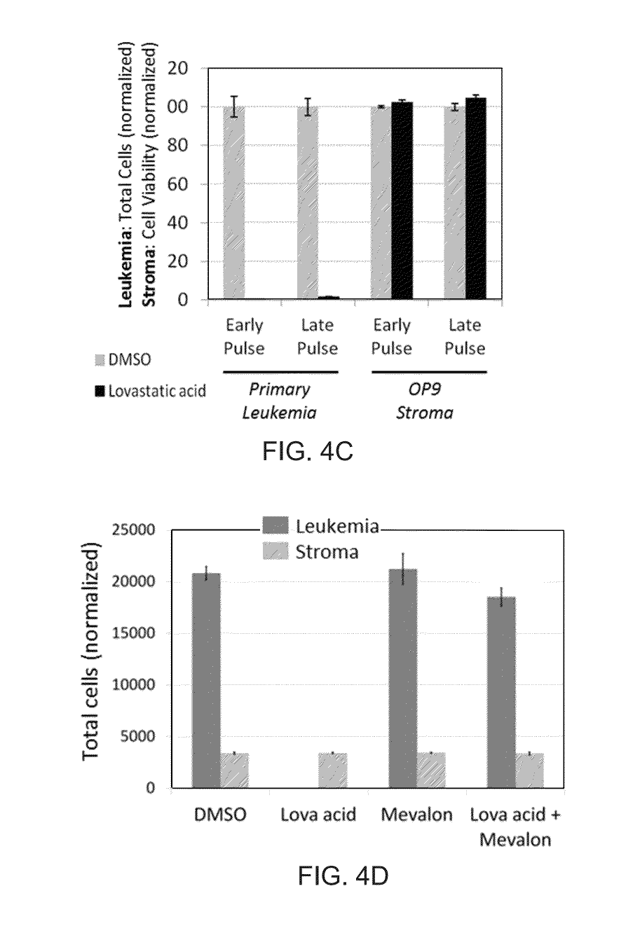

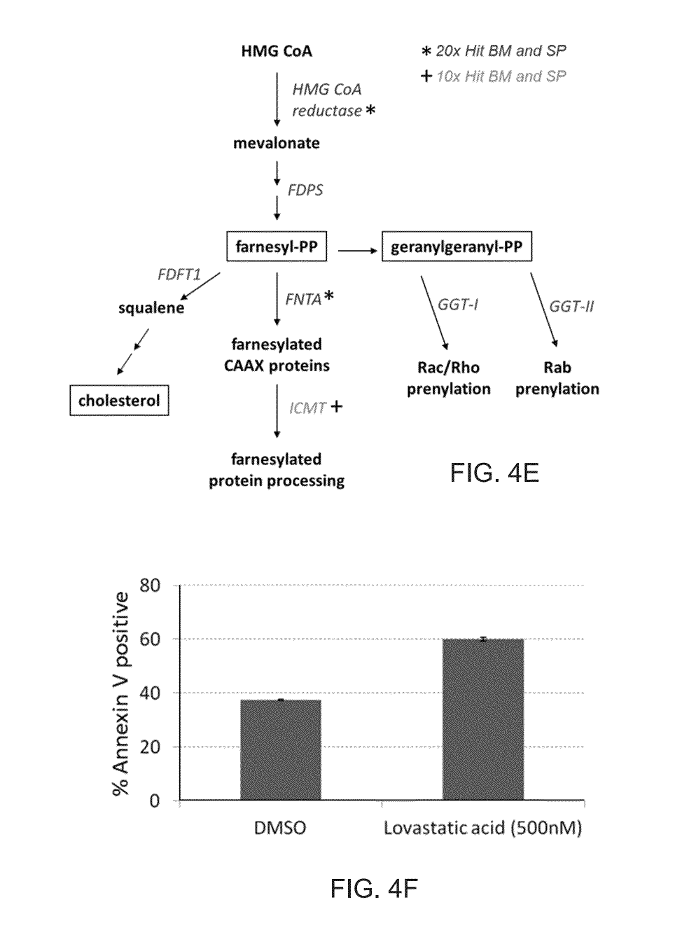

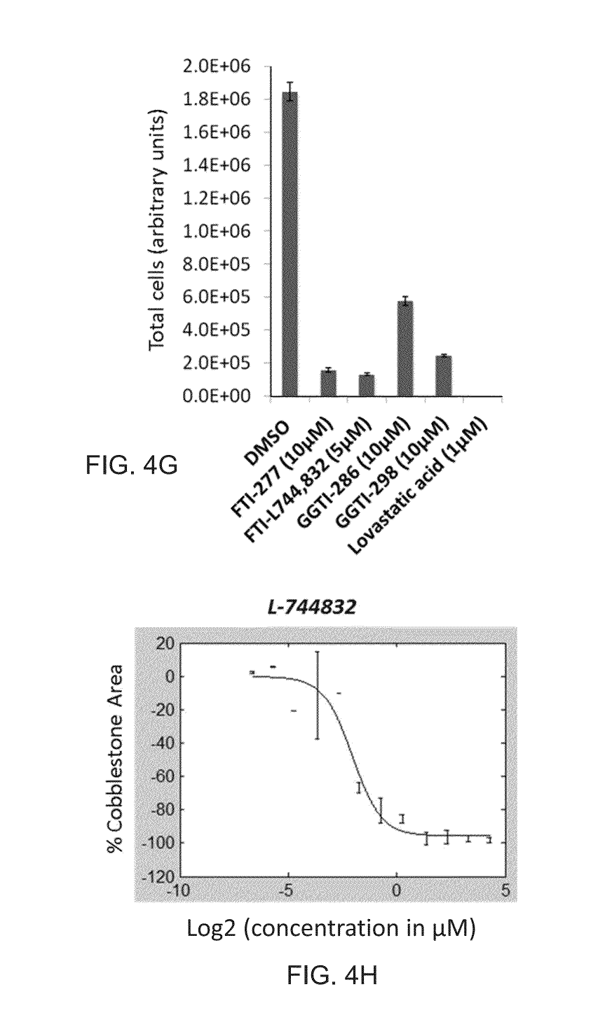

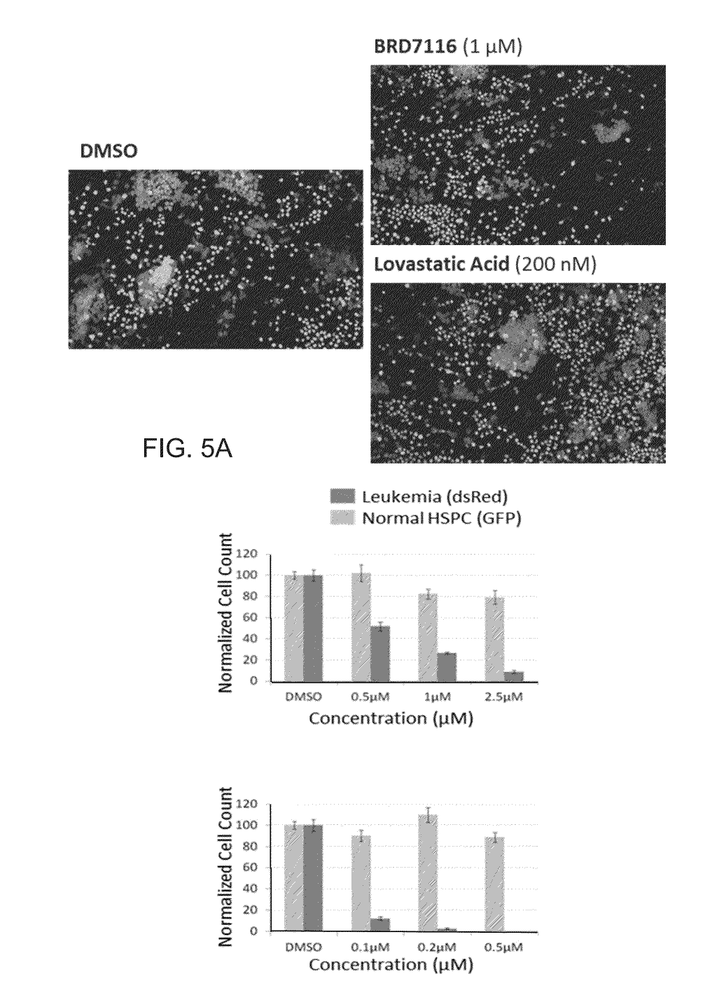

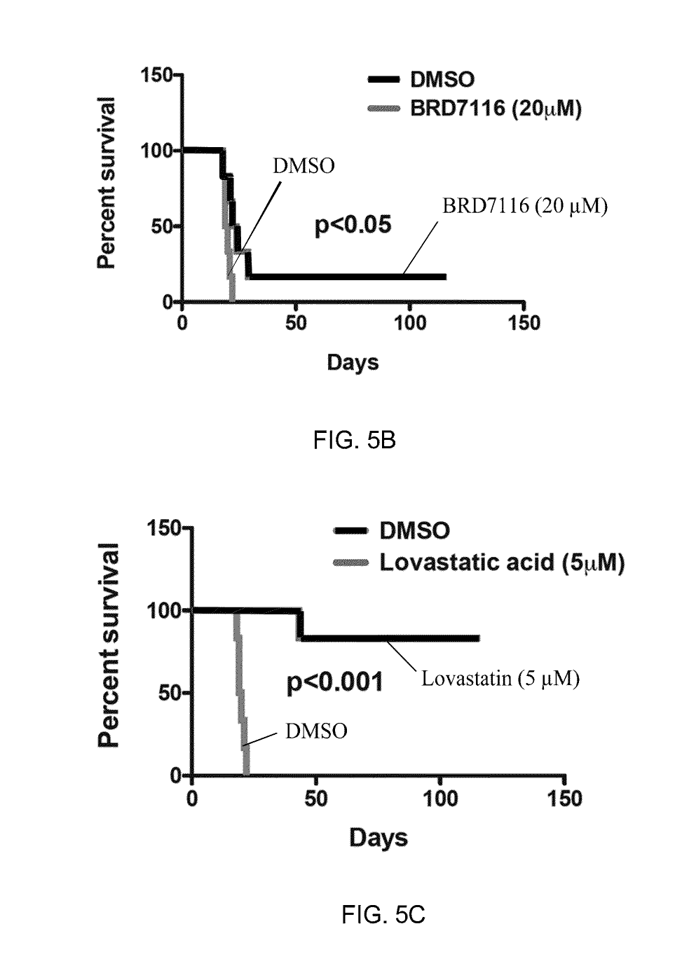

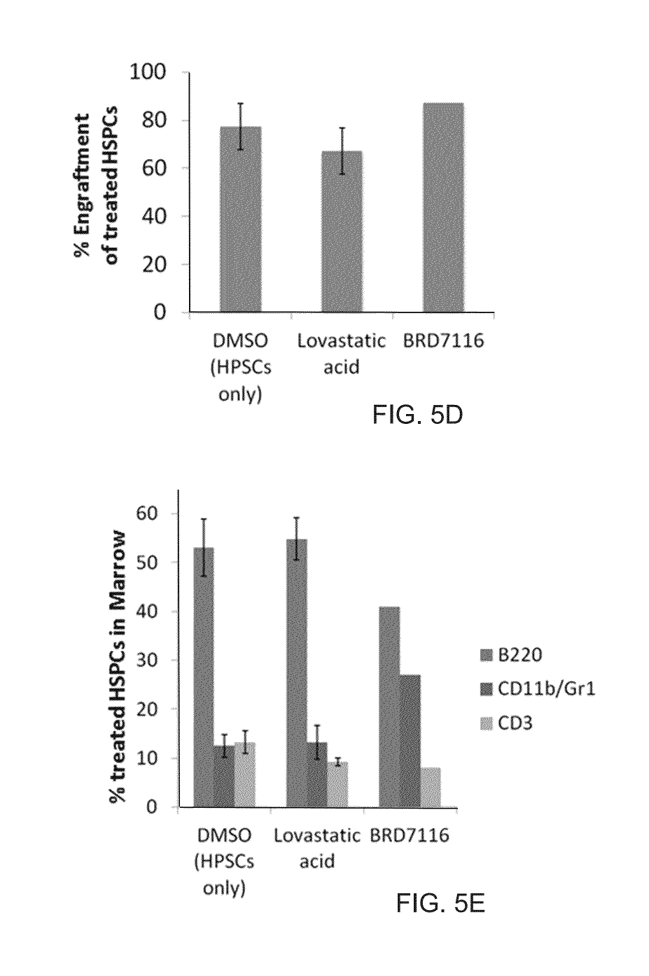

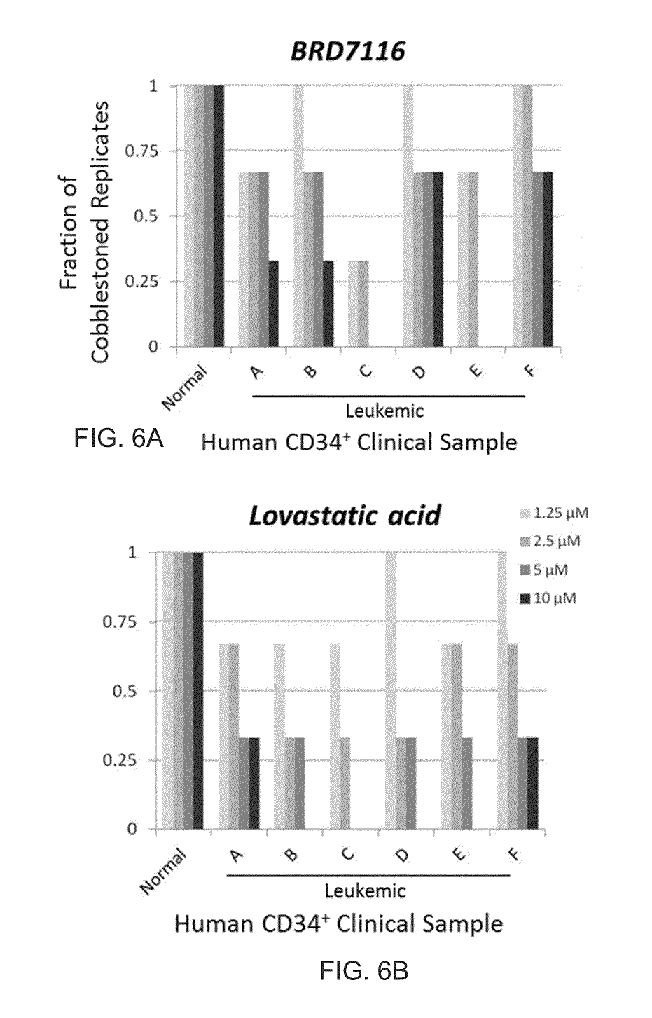

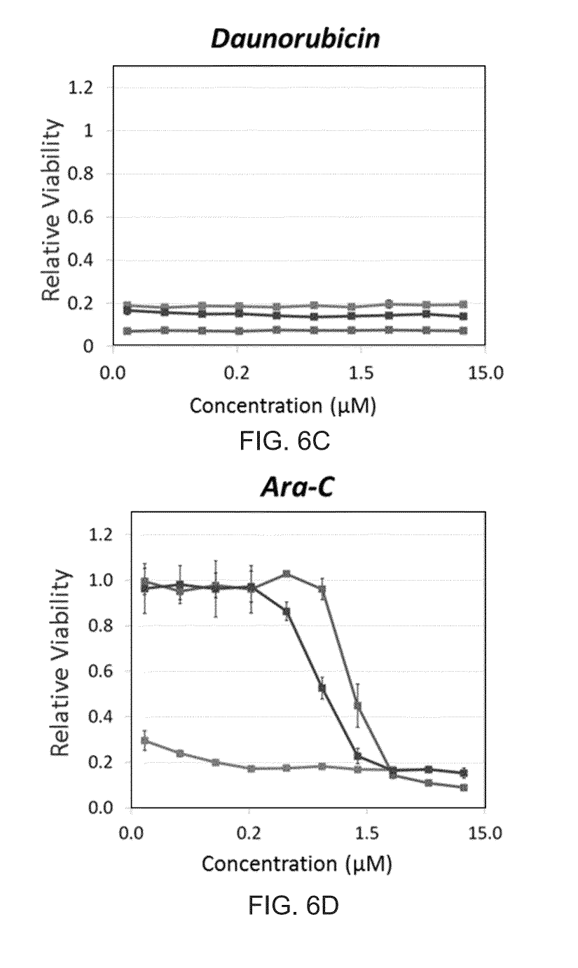

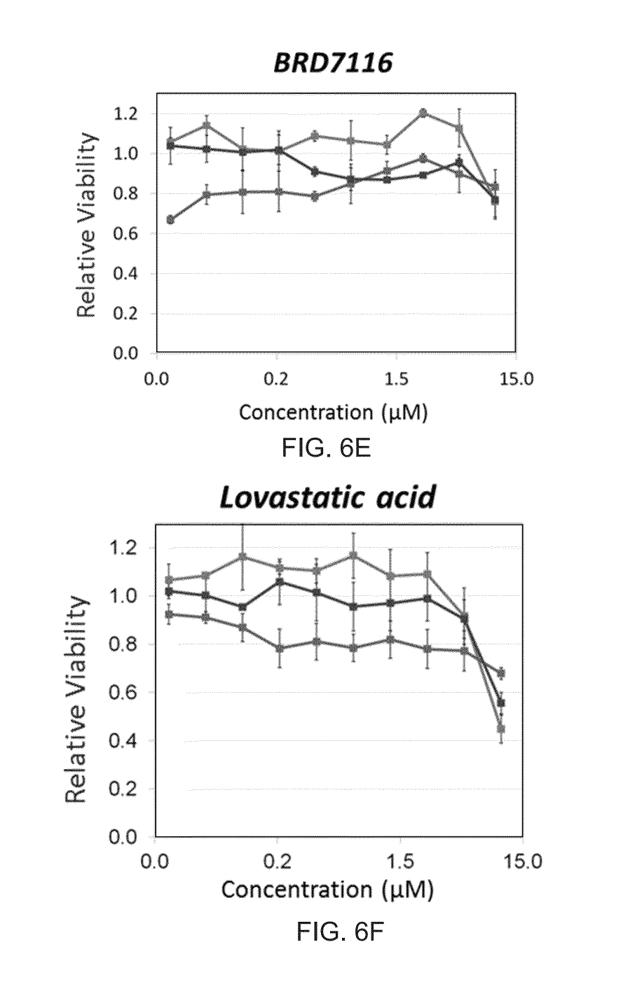

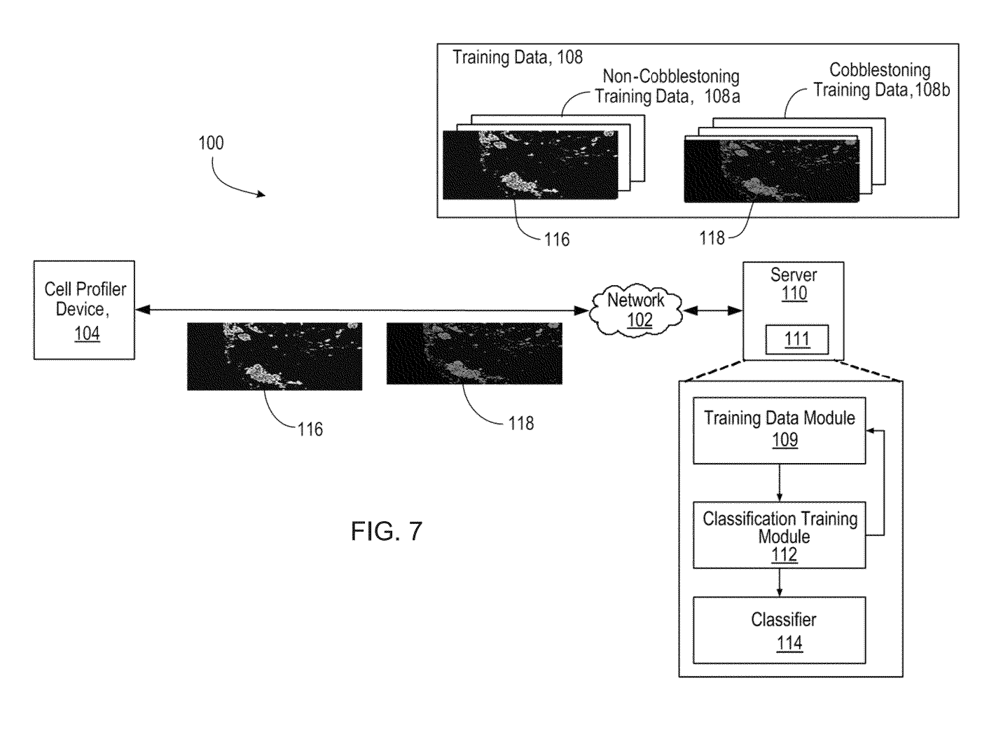

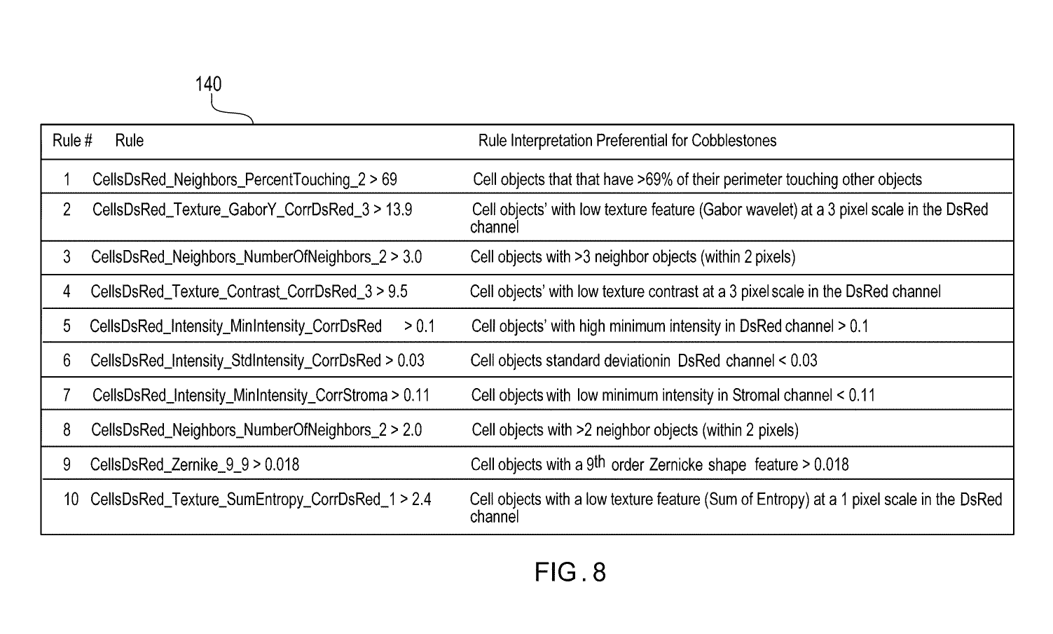

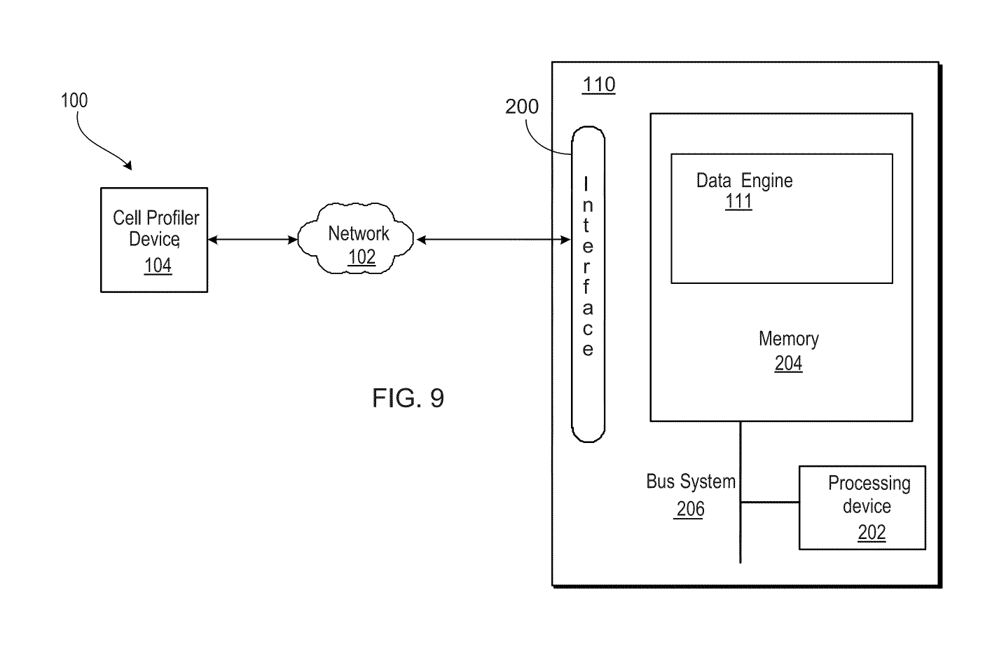

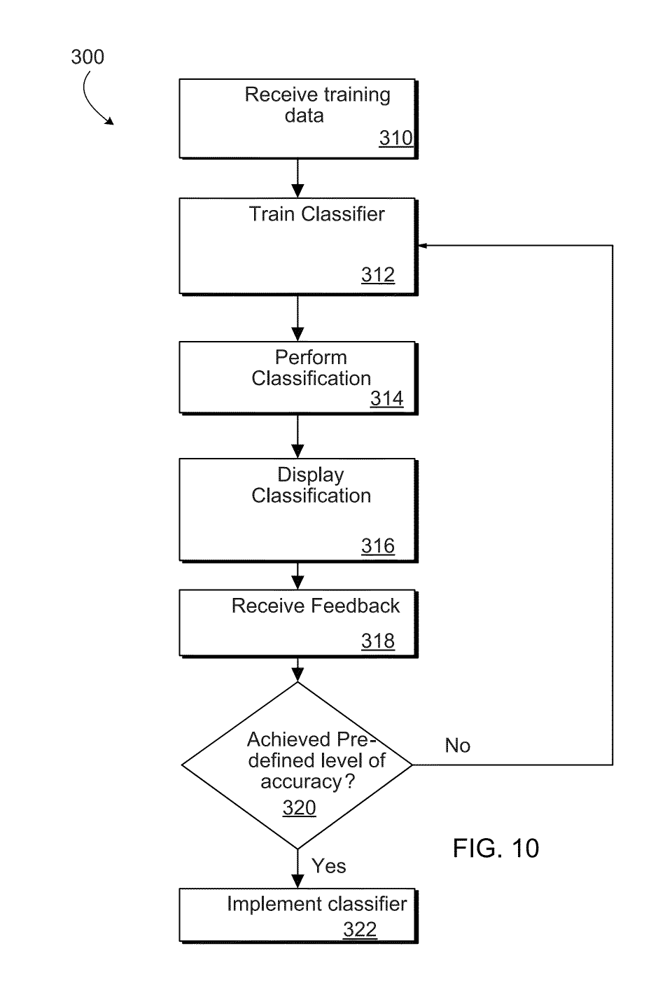

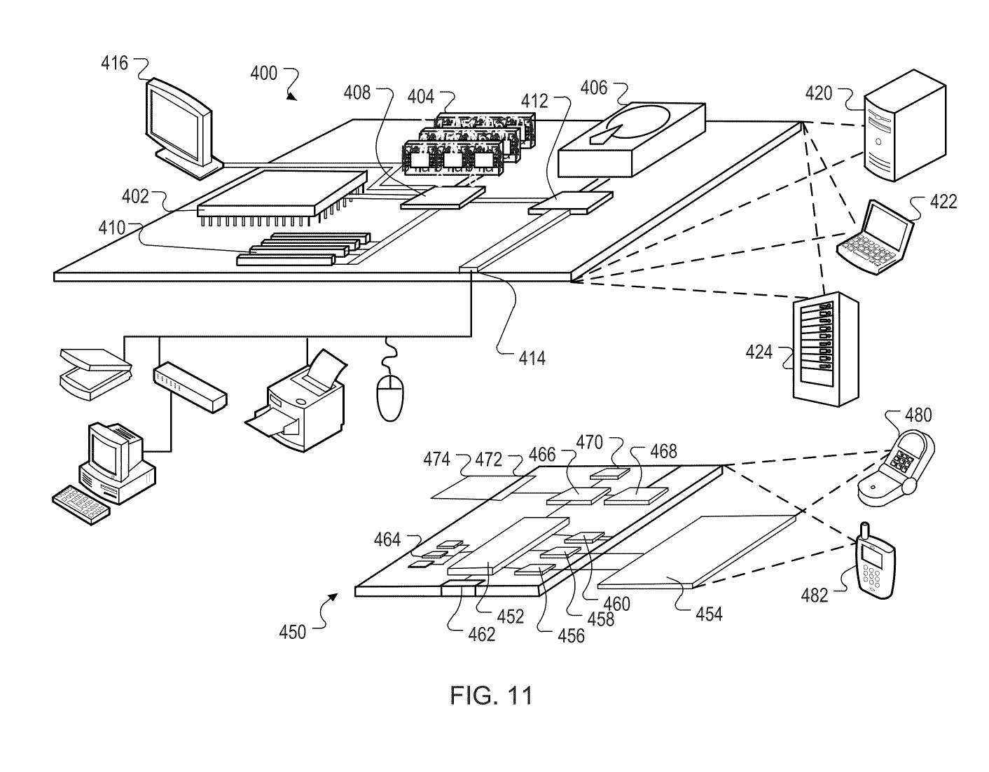

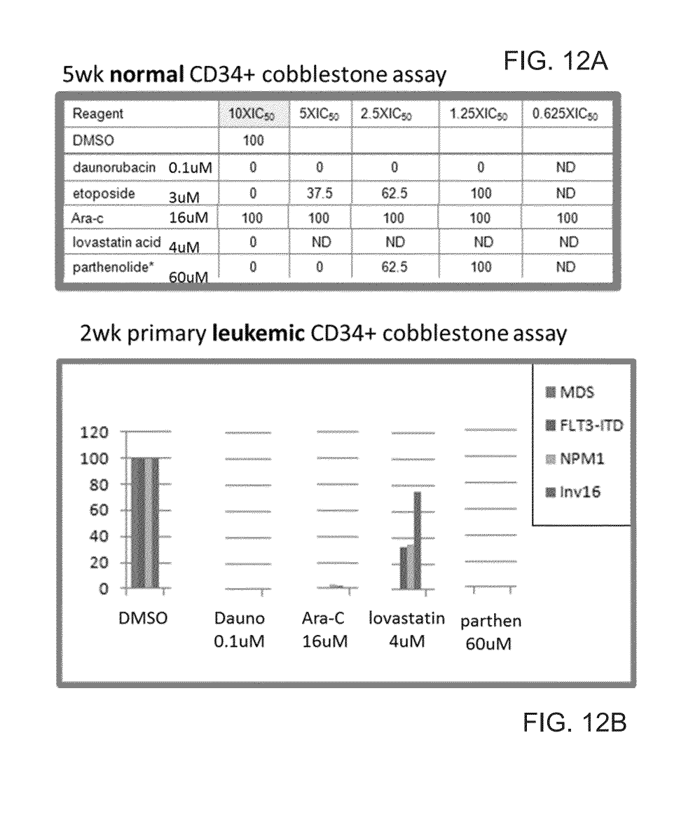

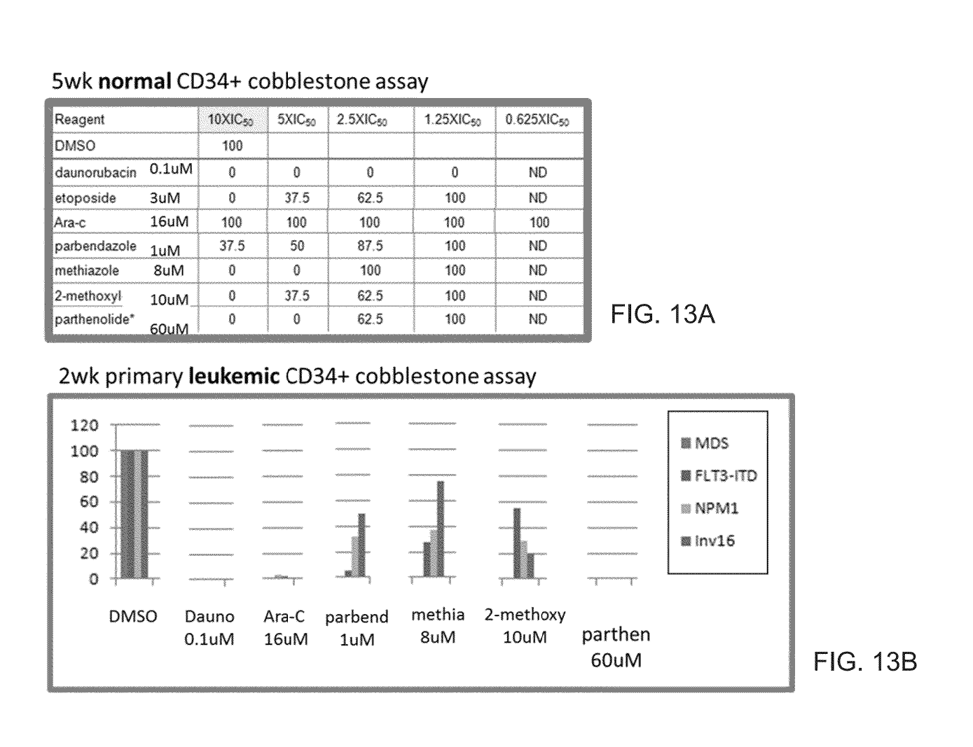

In general, the test samples are made by first plating the stromal cells, then later (e.g., 6, 12, 18, 24, or 26 hours later) plating the primary hematopoietic cells. In some embodiments, the cells are cultured together for a time before a test compound is added (see, e.g., FIG. 1J). Compounds that affect cobblestoning in this assay may be affecting either the stromal cells or the primary hematopoietic cells.

In some embodiments, after the stromal cells are plated, the test compound is added and the culture is maintained for some time before the test compound is washed off the stromal cells and the primary hematopoietic cells are added (see, e.g., FIG. 2B; this is referred to herein as a stromal pretreatment screen. Compounds that affect cobblestoning in this assay most likely affect the stromal cell support.

In some embodiments, the test and/or control samples include a number of cocultured cell populations, each of which is individually labelled with distinct fluorchromes, thus enabling individual evaluation. The effects on each individual cell population can be examined in its corresponding channel, e.g., using a cobblestone metric as described herein, or any other measure, e.g., cell proliferation, viability, cell cycle stage, confluence, inter alia.

The screening methods then include the detection of formation of cobblestoned areas, e.g., using the algorithms described herein. Those compounds that are present in a well that exhibits reduced cobblestone formation can be selected as candidate compounds for the treatment of leukemia. Since cobblestone formation, as discussed above, is associated with stem cell activity, those compounds have the potential to affect LSCs in vivo. In addition, the methods can be used to identify compounds that increase cobblestoning. Compounds that increase cobblestoning are useful in regenerative medicine (for example, compounds that increase cobblestoning in normal cell populations (e.g., control samples)).

The following describes one embodiment of a computer-implemented high-throughput screening method for identifying compounds that inhibit cobblestoning in this assay, e.g., from raw images of individual test samples (or portions thereof), and thus are candidate compounds for the treatment of leukemia.

FIG. 7 is a diagram of an example of a network environment 100 for training a classifier to identify compounds for the treatment of leukemia. Network environment 100 includes network 102, cell profiler device 104, and server 110.

Cell profiler device 104 can communicate with server 110 over network 102. Network environment 100 may include many thousands of data repositories and servers, which are not shown. Server 110 may include various data engines, including, e.g., data engine 111. Data engine 111 can exist as a single component or as one or more components, which can be distributed and coupled by network 102. In the example of FIG. 1, data engine 111 includes training data module 109, classification training module 112 and classifier 114.

In an example, cell profiler device 104 includes a device for receiving raw images of cells. From the raw images of the cells, cell profiler device 104 includes software for performing various techniques, including, e.g., performing illumination correction, measuring stromal coverage, identifying peaks, and identifying cell boundaries. Based on performance of these techniques, cell profiler device 104 measures features of the raw images, and subregions thereof. The measured features includes intensity, shape, neighbors, texture, and so forth.

In this example, the features are visually depicted in images, including, e.g., images 116, 118. Images 116, 118 include visual representation of features of cells to which a compound has been applied. A visualization of the features results from various masks and/or filters being applied to the combination of the compound and the cells, e.g., in a well.

In this example, various, different compounds are applied to the cells. Some of the compounds promote treatment of leukemia, e.g., as is evidenced by cobblestoning. As described in further detail below, data engine 111 identifies compounds that promote treatment of leukemia by generating classifier 114 to identify cobblestoning in images of features that result from various combinations of cells and compounds. In this example, an assay includes the combinations of cells and compounds.

In an example, cell profile device 104 transmits images 116, 118 to server 110. In response, server 110 generates a graphical user interface (not shown) for display of images 116, 118 to a user (not shown) of server 110. Following viewing of images 116, 118, the user inputs into server 110 data specifying whether each of images 116, 118 exhibits cobblestoning, including, e.g., an in vitro marker associated with leukemia cell health and self-renewal. Based on the input data, data engine 111 generates training data 108. Generally, training data includes data that is used in training a classifier.

In the example of FIG. 1, training data 108 includes non-cobblestoning training data 108a and cobblestoning training data 108b. Generally, non-cobblestoning training data 108a includes data (e.g., a set of images) in which cobblestoning is not exhibited. In this example, non-cobblestoning training data 108a includes image 116. Generally, cobblestoning training data 108b includes data (e.g., a set of images) in which cobblestoning is exhibited. In this example, cobblestoning training data 108b includes image 118.

In an example, training data module 109 is configured to obtain training data 108. Training data module 109 transmits training data 108 to classification training module 112. Classification training module 112 is configured to train classifier 114, e.g., using training data 108. Generally, classifier 114 is configured to classify items of data to a group associated with a feature. In an example, the feature may include cobblestoning. In this example, classifier 114 is configured to classify data into a cobblestoning group or into a non-cobblestoning group. Generally, a cobblestoning group includes a set of data associated with cobblestoning (e.g., a set of data that exhibits cobblestoning). Generally, a non-cobblestoning group includes a set of data not associated with cobblestoning. In the example of FIG. 1, classifier 114 includes a set of rules that are used in determining whether data exhibits cobblestoning.

Classification training module 112 develops classifier 114 based on an application of an interactive machine learning technique (e.g., an interactive machine learning algorithm) and a classification technique (e.g., a classification algorithm). Generally, an interactive machine learning technique includes a machine learning model that interactively queries an information source to obtain desired outputs at new data points.

In an example, classification training module 112 implements a classification technique in building classifier 114. Classification techniques include linear classifiers (e.g., a Naive Bayes classifier), quadratic classifiers, k-nearest neighbor classifiers, decision trees (e.g., random forests), neural networks, Bayesian networks, hidden Markov models, learning vector quantization classifiers, Boosting algorithms, and so forth.

In this example, classification training module 112 applies a classification technique to training data 108 to generate classifier 114 including one or more rules. In this example, training data 108 includes hundreds or thousands of images that have been classified, by users of server 110, as (i) exhibiting cobblestoning and belonging to a group of cobblestoning training data 108b, or (ii) not exhibiting cobblestoning and belonging to another group of non-cobblestoning training data 108a. In an example, data engine 111 identifies patterns in training data 108, including, e.g., patterns that are dependent on cobblestoning (e.g., patterns that are indicative of cobblestoning) and patterns that are independent of cobblestoning. Based on the patterns that are dependent on cobblestoning, data engine 111 generates one or more rules that characterize the patterns.

Based on the generated one or more rules, classification training module 112 presents the user with images that have been classified in accordance with the one or more rules. In this example, server 110 may be configured to access unclassified images, from cell profiler device 104, for use in testing an accuracy of classifier 114.

The user inputs, into server 110, additional information specifying an accuracy of the classifications based on the one or more rules. In an example, the user inputs information that is used by classification training model 112 to improve an accuracy of the one or more rules of classifier 114. In this example, the user corrects errors in classification of images.

The actions of applying classifier 114 to unclassified images, presenting results of the classification to the user, receiving feedback from the user, and using the feedback in re-training classifier 114 are repeated until classifier 114 achieves a pre-defined level of accuracy. For example, these actions may be repeated until classifier 114 achieves a ninety percent level of accuracy.

Once classifier 114 has achieved the pre-defined level of accuracy, server 110 applies classifier 114 to unclassified images (e.g., received from cell profiler device 104) to determine whether an image exhibits cobblestoning.

As previously described, classifier 114 includes various rules, as shown in table 140 in FIG. 8. In the example of FIG. 8, classifier 114 includes rules 1-10, e.g., which are based on features of cells in an image. In this example, the rules are based on features and/or patterns that are indicative of cobblestoning.

In an example, rule 1 specifies that data exhibits cobblestoning when cell objects have at least sixty-nine percent of a perimeter touching other objects in the data. Rule 2 specifies that data exhibits cobblestoning when cell objects exhibit a low texture feature. Rule 3 specifies that data exhibits cobblestoning when a cell object has more than a pre-defined number of neighbor objects within a particular proximity.

Rule 4 specifies that data exhibits cobblestoning when a cell object has low texture contrast at a three pixel scale, e.g., a channel marked by a particular dye—the DsRed channel. Rule 5 specifies that data exhibits cobblestoning when a cell object has a particular level of intensity in a DsRed channel. Rule 6 specifies that data exhibits cobblestoning when a cell object has a particular standard deviation in a DsRed channel. Rule 7 specifies that data exhibits cobblestoning when a cell object has low minimum intensity in a Stromal channel. Rule 8 specifies that data exhibits cobblestoning when a cell object has more than two neighbor objects within 2 pixels in an image. Rule 9 specifies that data exhibits cobblestoning when a cell object is associated with a pre-defined order Zernike shape feature that is greater than a pre-defined value. Rule 10 specifies that data exhibits cobblestoning when a cell object has a low texture feature at a one pixel scale in the DsRed channel.

FIG. 9 is a block diagram showing examples of components of network environment 100 for training classifier 114 to identify compounds for the treatment of leukemia. In the example of FIG. 9, images 116, 118, training data 108 and modules 109, 112, 114 of data engine 111 are not shown.

Network 102 can include a large computer network, including, e.g., a local area network (LAN), wide area network (WAN), the Internet, a cellular network, or a combination thereof connecting a number of mobile computing devices, fixed computing devices, and server systems. The network(s) may provide for communications under various modes or protocols, including, e.g., Transmission Control Protocol/Internet Protocol (TCP/IP), Global System for Mobile communication (GSM) voice calls, Short Message Service (SMS), Enhanced Messaging Service (EMS), or Multimedia Messaging Service (MMS) messaging, Code Division Multiple Access (CDMA), Time Division Multiple Access (TDMA), Personal Digital Cellular (PDC), Wideband Code Division Multiple Access (WCDMA), CDMA2000, or General Packet Radio System (GPRS), among others. Communication may occur through a radio-frequency transceiver. In addition, short-range communication may occur, including, e.g., using a Bluetooth, WiFi, or other such transceiver.

Server 110 can be a variety of computing devices capable of receiving data and running one or more services. In an example, server 110 can include a server, a distributed computing system, a desktop computer, a laptop, a cell phone, a rack-mounted server, and the like. Server 110 can be a single server or a group of servers that are at a same location or at different locations. Cell profiler device 104 and server 110 can run programs having a client-server relationship to each other. Although distinct modules are shown in the figures, in some examples, client and server programs can run on the same device.

Server 110 can receive data from cell profiler device 104 through input/output (I/O) interface 200. I/O interface 200 can be a type of interface capable of receiving data over a network, including, e.g., an Ethernet interface, a wireless networking interface, a fiber-optic networking interface, a modem, and the like. Server 110 also includes a processing device 202 and memory 204. A bus system 206, including, for example, a data bus and a motherboard, can be used to establish and to control data communication between the components of server 110.

Processing device 202 can include one or more microprocessors. Generally, processing device 202 can include an appropriate processor and/or logic that is capable of receiving and storing data, and of communicating over a network (not shown). Memory 204 can include a hard drive and a random access memory storage device, including, e.g., a dynamic random access memory, or other types of non-transitory machine-readable storage devices. As shown in FIG. 9, memory 204 stores computer programs that are executable by processing device 202. These computer programs include data engine 111. Data engine 111 can be implemented in software running on a computer device (e.g., server 110), hardware or a combination of software and hardware.

FIG. 10 is a flowchart showing an example process 300 for training classifier 114 to identify compounds for the treatment of leukemia. In FIG. 3, process 300 is performed on server 110 (and/or by data engine 111 on server 110).

In operation, training data module 109 receives (310) training data 108. As previously described, data engine 111 generates training data 108 based on a classification of images by the user. In this example, the user classifies images as exhibiting cobblestoning or as not exhibiting cobblestoning. Based on the user-specified classification, data engine 111 generates non-cobblestoning training data 108a and cobblestoning training data 108b.

Using training data 108, classification training module 112 trains (312) classifier 114. In an example, classification training module 112 applies a classification technique in generating classifier 114 from training data 108. Classification training module 112 tests an accuracy of classifier 114 by performing (314) classification on unclassified data. Classification training module 112 displays (316) for the user the classification. In an example, classification training module 112 generates a graphical user interface that when rendered on server 110 renders a visual representation of the classification.

In the example of FIG. 10, training data module 109 receives (318) feedback from the user. In this example, the feedback includes data indicative of a correctness of the classifications that were generated using classifier 114. Training data module 112 determines (320) whether classifier 114 has achieved a pre-defined level of accuracy, e.g., based on results of the feedback.

In an example, the pre-defined level of accuracy includes a predetermined, e.g., 70%, 80%, 90%, or greater, level of accuracy. In an example, training data module 109 determines that a level of accuracy of classifier 114 is less than the pre-defined level. In this example, actions 312, 314, 316, 318, 320 are repeated (e.g., periodically, iteratively, and so forth), until the level of accuracy of classifier 114 is equal to or greater than the pre-defined level. In another example, training data module 109 determines that a level of accuracy of classifier 114 exceeds the pre-defined level. In this example, data engine 111 implements (322) classifier 114. In an example, data engine 111 implements classifier 114 by applying classifier 114 to unclassified data. Based on application of classifier 114, data engine 111 classifies the data as belong to the cobblestoning group or as belonging to the non-cobblestoning group.

FIG. 11 shows an example of computer device 400 and mobile computer device 450, which can be used with the techniques described here. Computing device 400 is intended to represent various forms of digital computers, such as laptops, desktops, workstations, personal digital assistants, servers, blade servers, mainframes, and other appropriate computers. Computing device 450 is intended to represent various forms of mobile devices, such as personal digital assistants, cellular telephones, smartphones, and other similar computing devices. The components shown here, their connections and relationships, and their functions, are meant to be examples only, and are not meant to limit implementations of the techniques described and/or claimed in this document.

Computing device 400 includes processor 402, memory 404, storage device 406, high-speed interface 408 connecting to memory 404 and high-speed expansion ports 410, and low speed interface 412 connecting to low speed bus 414 and storage device 406. Each of components 402, 404, 406, 408, 410, and 412, are interconnected using various busses, and can be mounted on a common motherboard or in other manners as appropriate. Processor 402 can process instructions for execution within computing device 400, including instructions stored in memory 404 or on storage device 406 to display graphical data for a GUI on an external input/output device, such as display 416 coupled to high speed interface 408. In other implementations, multiple processors and/or multiple buses can be used, as appropriate, along with multiple memories and types of memory. Also, multiple computing devices 400 can be connected, with each device providing portions of the necessary operations (e.g., as a server bank, a group of blade servers, or a multi-processor system).

Memory 404 stores data within computing device 400. In one implementation, memory 404 is a volatile memory unit or units. In another implementation, memory 404 is a non-volatile memory unit or units. Memory 404 also can be another form of computer-readable medium, such as a magnetic or optical disk.

Storage device 406 is capable of providing mass storage for computing device 400. In one implementation, storage device 406 can be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in a data carrier. The computer program product also can contain instructions that, when executed, perform one or more methods, such as those described above. The data carrier is a computer- or machine-readable medium, such as memory 404, storage device 406, memory on processor 402, and the like.

High-speed controller 408 manages bandwidth-intensive operations for computing device 400, while low speed controller 412 manages lower bandwidth-intensive operations. Such allocation of functions is an example only. In one implementation, high-speed controller 408 is coupled to memory 404, display 416 (e.g., through a graphics processor or accelerator), and to high-speed expansion ports 410, which can accept various expansion cards (not shown). In the implementation, low-speed controller 412 is coupled to storage device 406 and low-speed expansion port 414. The low-speed expansion port, which can include various communication ports (e.g., USB, Bluetooth®, Ethernet, wireless Ethernet), can be coupled to one or more input/output devices, such as a keyboard, a pointing device, a scanner, or a networking device such as a switch or router, e.g., through a network adapter.

Computing device 400 can be implemented in a number of different forms, as shown in the figure. For example, it can be implemented as standard server 420, or multiple times in a group of such servers. It also can be implemented as part of rack server system 424. In addition or as an alternative, it can be implemented in a personal computer such as laptop computer 422. In some examples, components from computing device 400 can be combined with other components in a mobile device (not shown), such as device 450. Each of such devices can contain one or more of computing device 400, 450, and an entire system can be made up of multiple computing devices 400, 450 communicating with each other.

Computing device 450 includes processor 452, memory 464, an input/output device such as display 454, communication interface 466, and transceiver 468, among other components. Device 450 also can be provided with a storage device, such as a microdrive or other device, to provide additional storage. Each of components 450, 452, 464, 454, 466, and 468, are interconnected using various buses, and several of the components can be mounted on a common motherboard or in other manners as appropriate.

Processor 452 can execute instructions within computing device 450, including instructions stored in memory 464. The processor can be implemented as a chipset of chips that include separate and multiple analog and digital processors. The processor can provide, for example, for coordination of the other components of device 450, such as control of user interfaces, applications run by device 450, and wireless communication by device 450.

Processor 452 can communicate with a user through control interface 458 and display interface 456 coupled to display 454. Display 454 can be, for example, a TFT LCD (Thin-Film-Transistor Liquid Crystal Display) or an OLED (Organic Light Emitting Diode) display, or other appropriate display technology. Display interface 456 can comprise appropriate circuitry for driving display 454 to present graphical and other data to a user. Control interface 458 can receive commands from a user and convert them for submission to processor 452. In addition, external interface 462 can communicate with processor 442, so as to enable near area communication of device 450 with other devices. External interface 462 can provide, for example, for wired communication in some implementations, or for wireless communication in other implementations, and multiple interfaces also can be used.

Memory 464 stores data within computing device 450. Memory 464 can be implemented as one or more of a computer-readable medium or media, a volatile memory unit or units, or a non-volatile memory unit or units. Expansion memory 474 also can be provided and connected to device 450 through expansion interface 472, which can include, for example, a SIMM (Single In Line Memory Module) card interface. Such expansion memory 474 can provide extra storage space for device 450, or also can store applications or other data for device 450. Specifically, expansion memory 474 can include instructions to carry out or supplement the processes described above, and can include secure data also. Thus, for example, expansion memory 474 can be provide as a security module for device 450, and can be programmed with instructions that permit secure use of device 450. In addition, secure applications can be provided via the SIMM cards, along with additional data, such as placing identifying data on the SIMM card in a non-hackable manner.

The memory can include, for example, flash memory and/or NVRAM memory, as discussed below. In one implementation, a computer program product is tangibly embodied in a data carrier. The computer program product contains instructions that, when executed, perform one or more methods, such as those described above. The data carrier is a computer- or machine-readable medium, such as memory 464, expansion memory 474, and/or memory on processor 452, that can be received, for example, over transceiver 468 or external interface 462.

Device 450 can communicate wirelessly through communication interface 466, which can include digital signal processing circuitry where necessary. Communication interface 466 can provide for communications under various modes or protocols, such as GSM voice calls, SMS, EMS, or MMS messaging, CDMA, TDMA, PDC, WCDMA, CDMA2000, or GPRS, among others. Such communication can occur, for example, through radio-frequency transceiver 468. In addition, short-range communication can occur, such as using a Bluetooth®, WiFi, or other such transceiver (not shown). In addition, GPS (Global Positioning System) receiver module 470 can provide additional navigation- and location-related wireless data to device 450, which can be used as appropriate by applications running on device 450.

Device 450 also can communicate audibly using audio codec 460, which can receive spoken data from a user and convert it to usable digital data. Audio codec 460 can likewise generate audible sound for a user, such as through a speaker, e.g., in a handset of device 450. Such sound can include sound from voice telephone calls, can include recorded sound (e.g., voice messages, music files, and the like) and also can include sound generated by applications operating on device 450.

Computing device 450 can be implemented in a number of different forms, as shown in the figure. For example, it can be implemented as cellular telephone 480. It also can be implemented as part of smartphone 482, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions.

To provide for interaction with a user, the systems and techniques described here can be implemented on a computer having a display device (e.g., a CRT (cathode ray tube) or LCD (liquid crystal display) monitor) for displaying data to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

In some implementations, the engines described herein can be separated, combined or incorporated into a single or combined engine. The engines depicted in the figures are not intended to limit the systems described here to the software architectures shown in the figures.

In some embodiments, the methods include comparing the effect of the compound on cobblestoning in co-cultures (test samples) comprising leukemic primary hematopoietic cells cells to the effect on cobblestoning in co-cultures (test samples) comprising normal (i.e., non-leukemic) primary hematopoietic cells, and selecting those compounds that affect cobblestoning only in the leukemic samples, and do not substantially affect cobblestoning in the samples comprising normal primary hematopoietic cells.

A test compound that has been screened by a method described herein and determined to inhibit cobblestoning can be considered a candidate compound for the treatment of leukemia. Test compounds identified as candidate therapeutic compounds can be further screened by administration to an animal model of leukemia, as known in the art or described herein. The animal can be monitored for an improvement in a parameter of leukemia, e.g., a parameter related to clinical outcome such as the presence or level of abnormal cells associated with the leukemia, survival time, time to relapse, or severity of associated symptoms, can be considered a candidate therapeutic agent.

A candidate compound that has been screened, e.g., in an in vivo model of leukemia and determined to have a desirable effect on one or more parameters, e.g., a parameter related to clinical outcome such as the presence or level of abnormal cells associated with the leukemia, survival time, time to relapse, or severity of associated symptoms, can be considered a candidate therapeutic agent. Candidate therapeutic agents, once screened in a clinical setting (e.g., a clinical trial), are therapeutic agents. Candidate compounds, candidate therapeutic agents, and therapeutic agents can be optionally optimized and/or derivatized, and formulated with physiologically acceptable excipients to form pharmaceutical compositions.

Thus, test compounds identified as “hits” (e.g., test compounds that inhibit cobblestoning) in a first screen can be selected and systematically altered, e.g., using rational design, to optimize binding affinity, avidity, specificity, or other parameter. Such optimization can also be screened for using the methods described herein. Thus, in one embodiment, the invention includes screening a first library of compounds using a method known in the art and/or described herein, identifying one or more hits in that library, subjecting those hits to systematic structural alteration to create a second library of compounds structurally related to the hit, and screening the second library using the methods described herein.

Test compounds identified as hits can be considered candidate therapeutic compounds, useful in treating leukemia. A variety of techniques useful for determining the structures of “hits” can be used in the methods described herein, e.g., NMR, mass spectrometry, gas chromatography equipped with electron capture detectors, fluorescence and absorption spectroscopy. Thus, the invention also includes compounds identified as “hits” by the methods described herein, and methods for their administration and use in the treatment, prevention, or delay of development or progression of a disorder described herein.

Methods of Treating Leukemia

Provided herein are compounds useful for treating leukemia, for example, acute myelogenous leukemia (AML). Also provided herein are methods and materials for using such compounds to reduce the number of leukemia cells in a patient, e.g., a patient in remission. For example, a compound provided herein can be used to reduce the number of leukemia stem cells in a patient. In some cases, a patient, e.g., a patient in remission, can be treated with a compound provided herein (e.g., a statin or a compound of formula (I)-(VIII)). Further provided herein is a method of inhibiting growth of leukemia cells in a patient, e.g., a patient in remission, by administration of a compound provided herein.

The methods provided herein include methods for the treatment of leukemia in a patient. In some embodiments, the leukemia is designated as acute or chronic. Generally, the methods include administering a therapeutically effective amount of a compound (i.e., active ingredient) as described herein (e.g., a statin and/or a compound o of formulas (I)-(VIII)), to a patient who is in need of, or who has been determined to be in need of, such treatment. For example, a patient can be identified as actively suffering from leukemia or as being in remission. In some embodiments, e.g., where the patient has active disease (i.e., is not in remission), the methods include administering a compound described herein plus another treatment, e.g., a chemotherapy or radiation treatment as known in the art, e.g., as described herein.

As used in this context, to “treat” means to ameliorate at least one symptom of the leukemia. In some cases, treatment with a compound provided herein can result in a reduction of the number of leukemic cells in the patient. Administration of a therapeutically effective amount of a compound described herein for the treatment of leukemia can also result in an inhibition of the growth of leukemic cells in the patient.

Preferably, the compounds provided herein can exhibit a preference for inhibition in growth and the reduction of leukemic cells over other cells present near or in the environment surrounding the leukemic cells. For example, in some embodiments, a compound provided herein can exhibit a preference for killing (or inhibiting the growth of) leukemic stem cells (LSCs) over non-stem leukemic cells (e.g., differentiated leukemia cells). In some embodiments, a compound provided herein can exhibit a preference for killing or inhibiting the growth of leukemic stem cells over stromal support cells and/or normal, primary hematopoietic stem and progenitor cells (HSCs).

As described above, leukemia can be classified by how quickly it progresses. Acute leukemia is fast-growing and can overrun the body within a few weeks or months. By contrast, chronic leukemia is slow-growing and typically progressively worsens over years.

The blood-forming (hematopoietic) cells of acute leukemia remain in an immature state, so they reproduce and accumulate very rapidly. In chronic leukemia, the blood-forming cells eventually mature, or differentiate, but they are not “normal.” They remain in the bloodstream much longer than normal white blood cells, and they are unable to combat infection well. Leukemia cells include a number of white blood cells such as lymphocytes (immune system cells), granulocytes (bacteria-destroying cells), and monocytes (macrophage-forming cells). The type of cell that is multiplying contributes to the classification of the disease. For example, if the abnormal white blood cells are primarily granulocytes or monocytes, the leukemia is categorized as myelogenous, or myeloid, leukemia. On the other hand, if the abnormal blood cells arise from bone marrow lymphocytes, the cancer is called lymphocytic leukemia. Other cancers, known as lymphomas, develop from lymphocytes within the lymph nodes, spleen, and other organs. Such cancers do not originate in the bone marrow and have a biological behavior that is different from lymphocytic leukemia.

In some embodiments, a leukemia cell is a leukemia stem cell (LSC). These cells are believed to be responsible for disease progression and for resistance to chemotherapeutic drugs. LSCs have a phenotype similar to that of a hematopoietic progenitor cell, which differs from the normal progenitor cells in a number of ways; in some embodiments, e.g., the leukemia stem cell has acquired an activated β-catenin pathway. As a result, the LSCs have acquired the proliferative and self-renewal capacity that is normally restricted to hematopoietic stem cells. For example, in CML, the LSCs responsible for disease progression are phenotypically similar to granulocyte/macrophage progenitor cells.

The compounds provided herein can also be administered in combination with other known methods of treating leukemia, for example by chemotherapy or irradiation. Thus, there is further provided a method of treating leukemia comprising administering a therapeutically effective amount of a compound provided herein, or a pharmaceutically acceptable salt form thereof, to a patient in need of such treatment, wherein an effective amount of at least one further cancer chemotherapeutic agent is administered to the patient. In some embodiments, an additional chemotherapeutic agent can be useful for targeting and killing differentiated leukemia cells. Examples of suitable chemotherapeutic agents include any of the agents shown in Table A, as well as AMD3100, CD44 agonism, abarelix, aldesleukin, alemtuzumab, alitretinoin, allopurinol, altretamine, anastrozole, arsenic trioxide, asparaginase, azacitidine, bevacizumab, bexarotene, bleomycin, bortezombi, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, epirubicin, erlotinib, estramustine, etoposide phosphate, etoposide, exemestane, fentanyl citrate, filgrastim, floxuridine, fludarabine, fluorouracil, fulvestrant, gefitinib, gemcitabine, gemtuzumab ozogamicin, goserelin acetate, histrelin acetate, ibritumomab tiuxetan, idarubicin, ifosfamide, imatinib mesylate, interferon alfa 2a, irinotecan, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, oxaliplatin, paclitaxel, pamidronate, panitumumab, pegaspargase, pegfilgrastim, pemetrexed disodium, pentostatin, pipobroman, plicamycin, procarbazine, quinacrine, rasburicase, rituximab, sorafenib, streptozocin, sunitinib, sunitinib maleate, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, vorinostat, and zoledronate. In some embodiments, the additional treatment is as shown in Table A.

Also provided is a method of treating leukemia comprising administering a therapeutically effective amount of a compound provided herein, or a pharmaceutically acceptable salt thereof, to a patient in need of such treatment, wherein an effective amount of ionizing radiation is also administered to the patient. In these methods, the further cancer therapeutic agent and/or the ionizing radiation may be administered concomitantly and/or non-concomitantly with the compound provided herein.

A compound provided herein, including a pharmaceutically acceptable salt form thereof, can be purchased from commercial sources or can be prepared using methods known to those skilled in the art of organic synthesis. See, for example, Morton, D. et al., Angew. Chem. Int. Ed. 2009, 48, 104-109; Schrieber, S. L., Science 1964, 287, 1964-1969; and Marcaurelle, L. A. et al., JACS 2010, 132, 16962-16976.

Statins

A compound provided herein can be a statin, or a prodrug, acid, or salt form thereof. Statins are a class of medications that have been shown to be effective in lowering human total cholesterol (TC) and low density lipoprotein (LDL) levels in hyperlipidemic patients. By reducing the amount of cholesterol synthesized by the cell, through inhibition of the HMG Co-A Reductase gene (HMGCR), statins initiate a cycle of events that culminates in the increase of LDL uptake by liver cells.

The essential structural components of all statins are a dihydroxyheptanoic acid unit and a ring system with different substituents. The statin pharmacophore is a modified hydroxyglutaric acid component, which is structurally similar to the endogenous substrate HMG CoA and the mevaldyl CoA transition state intermediate:

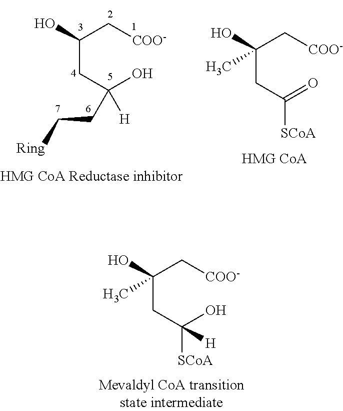

The statin pharmacophore binds to the same active site as the substrate HMG-CoA and inhibits the HMGCR enzyme. It has also been shown that the HMGCR is stereoselective and as a result statins have a 3R,5R stereochemistry.

Examples of statins include atorvastatin, cerivastatin, fluvastatin, lovastatin, mevastatin, pitavastatin, pravastatin, rosuvastatin, and simvastatin. In some embodiments, a statin provided herein is in an acid form. For example, an acid form of a statin can include atorvastatic acid, cerivastatic acid, fluvastatic acid, lovastatic acid, mevastatic acid, pitavastatic acid, pravastatic acid, rosuvastatic acid, and simvastatic acid. In some embodiments, a statin is provided in a pharmaceutically acceptable salt form. In some embodiments, a statin is cerivastatin, fluvastatin, or an acid form thereof. For example, a statin used in the methods provided herein can be fluvastatin.

The known statins differ structurally with respect to their ring structure and substituents. These differences in structure can affect the pharmacological properties of the statins, such as: affinity for the active site of the HMGR; rates of entry into hepatic and non-hepatic tissues; availability in the systemic circulation for uptake into non-hepatic tissues; and routes and modes of metabolic transformation and elimination.

Statins can be grouped into two groups according to their structure. Type 1 statins include a substituted decalin-ring structure. Examples of type 1 statins include mevastatin, lovastatin, pravastatin, and simvastatin. Type 2 statins, on the other hand, are fully synthetic and have larger groups linked to the HMG-like moiety. One of the main differences between the type 1 and type 2 statins is the replacement of the butyryl group of type 1 statins by the fluorophenyl group of type 2 statins. The fluorophenyl group is thought to be responsible for additional polar interactions that cause tighter binding to the HMGCR enzyme. Examples of type 2 statins include fluvastatin, cerivastatin, atorvastatin, and rosuvastatin.

Compounds of Formula (I)

In some embodiments, a compound provided herein can be a compound of formula (I):

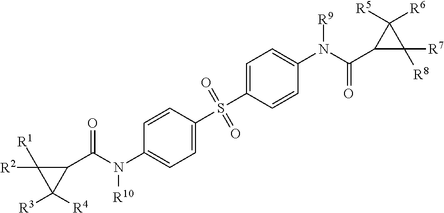

or a pharmaceutically acceptable salt form thereof,
wherein:
R¹, R², R³, R⁴, R⁵, R⁶, R⁷, and R⁸ are independently selected from the group consisting of: hydrogen, C₁₋₆ alkyl, C₁₋₆ alkenyl, and C₁₋₆ alkynyl; and
R⁹ and R¹⁰ are independently selected from the group consisting of: hydrogen and C₁₋₆ alkyl.

In some embodiments, R¹, R², R³, R⁴, R⁵, R⁶, R⁷, and R⁸ are independently a C₁₋₆ alkyl. For example, R¹, R², R³, R⁴, R⁵, R⁶, R⁷, and R⁸ can be CH₃. In some embodiments, R⁹ and R¹⁰ are hydrogen.

A non-limiting example of a compound of formula (I) includes:

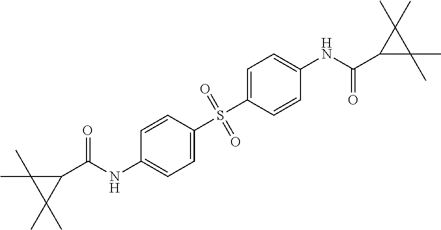

or a pharmaceutically acceptable salt form thereof.

Compounds of Formula (II)

Also provided herein are compounds of formula (II):

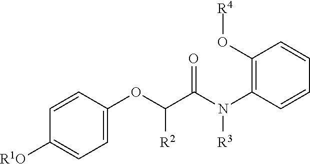

or a pharmaceutically acceptable salt form thereof,
wherein:
R¹, R³, and R⁴ are independently selected from the group consisting of: hydrogen and C₁₋₆ alkyl; and
R² is selected from the group consisting of: hydrogen, C₁₋₆ alkyl, C₁₋₆ alkenyl, and C₁₋₆ alkynyl.

In some embodiments, R¹ and R⁴ are independently a C₁₋₆ alkyl. For example, R¹ can be CH₃ and R⁴ can be CH₂CH₃. In some embodiments, R² is a C₁₋₆ alkyl. For example, R² can be CH₃. In some embodiments, R³ is hydrogen.

A non-limiting example of a compound of formula (II) is:

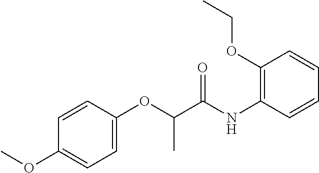 [SMILES: CCOc1ccccc1NC(=O)C(C)Oc1ccc(OC)cc1]

or a pharmaceutically acceptable salt form thereof.

Compounds of Formula (III)

Further provided herein are compounds of formula (III):

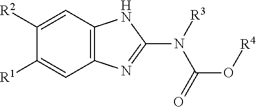

or a pharmaceutically acceptable salt form thereof,
wherein:
R¹ and R² are independently selected from the group consisting of hydrogen, C₁₋₆ alkyl,

C₁₋₆ alkenyl, C₁₋₆ alkynyl, OR⁵, C(O)R⁵, SR⁶, S(O)₂R⁵, carbocyclyl, heterocyclyl, aryl, and heteroaryl;
R³ and R⁴ are independently selected from the group consisting of: hydrogen and C₁₋₆ alkyl; and
each R⁵ is independently selected from the group consisting of: hydrogen, C₁₋₆ alkyl, C₁₋₆ alkenyl, C₁₋₆ alkynyl, carbocyclyl, heterocyclyl, aryl, and heteroaryl.

In some embodiments, one of R¹ and R² is hydrogen. In some embodiments, R⁴ is a C₁₋₆ alkyl. For example, R⁴ is CH₃. In some embodiments, R³ is hydrogen.

Non-limiting examples of a compound of formula (III) include:

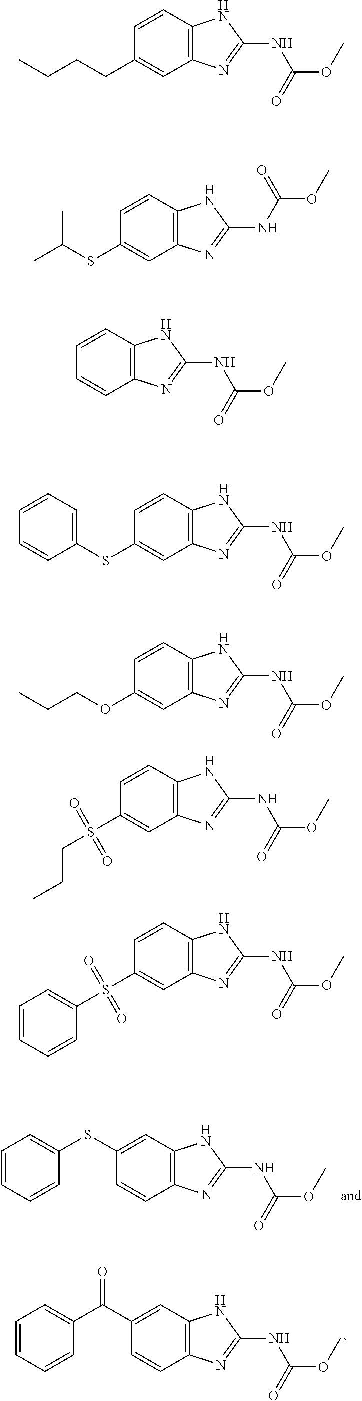

or a pharmaceutically acceptable salt form thereof.

Compounds of Formula (VI)

In some embodiments, a compound provided herein is a compound of formula (IV):

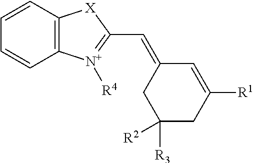

or a pharmaceutically acceptable salt form thereof,
wherein:
X is selected from S and O;
R¹, R² and R³ are independently selected from the group consisting of: hydrogen, C₁₋₆ alkyl, C₁₋₆ alkenyl, and C₁₋₆ alkynyl; and
R⁴ is selected from the group consisting of: hydrogen and C₁₋₆ alkyl.

In some embodiments, R¹ and R² are independently a C₁₋₆ alkyl. For example, R¹ and R² can be CH₃. In some embodiments, R³ is selected from the group consisting of hydrogen and C₁₋₆ alkyl (e.g., CH₃). In some embodiments, R⁴ is a C₁₋₆ alkyl. For example, R⁴ can be CH₂CH₃.

Non-limiting examples of a compound of formula (IV) include:

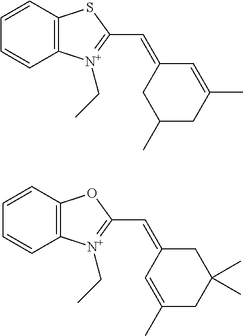 [SMILES: CC[n+]1c(/C=C2/C=C(C)CC(C)C2)sc2ccccc21 ; CC[n+]1c(/C=C2\C=C(C)CC(C)(C)C2)oc2ccccc21]

or a pharmaceutically acceptable salt form thereof.

Compounds of Formula (V)

Also provided herein are compounds of formula (V):

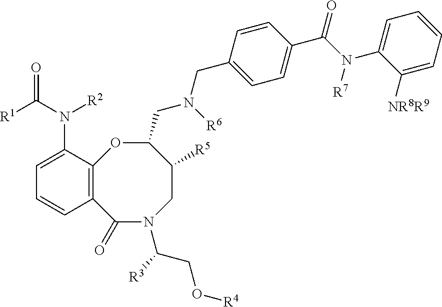

or a pharmaceutically acceptable salt form thereof,
wherein:
R¹ is selected from the group consisting of hydrogen, C₁₋₆ alkyl, C₁₋₆ alkenyl, C₁₋₆ alkynyl, NR¹⁰R¹¹, carbocyclyl, heterocyclyl, aryl, and heteroaryl;
R³ and R⁵ are independently selected from the group consisting of: hydrogen, C₁₋₆ alkyl, C₁₋₆ alkenyl, and C₁₋₆ alkynyl;
R², R⁴, R⁶, R⁷, R⁸, and R⁹ are independently selected from the group consisting of: hydrogen and C₁₋₆ alkyl; and
R¹⁰ and R¹¹ are independently selected from the group consisting of: hydrogen, C₁₋₆ alkyl, C₁₋₆ alkenyl, C₁₋₆ alkynyl, carbocyclyl, heterocyclyl, aryl, and heteroaryl.

In some embodiments, R¹ is selected from the group consisting of NR¹⁰R¹¹ and carbocyclyl. In some embodiments, R¹⁰ is hydrogen and R¹¹ is an aryl. In some embodiments, R³ and R⁵ are independently a C₁₋₆ alkyl. For example, R³ and R⁵ are CH₃. In some embodiments, R², R⁴, R⁷, R⁸, and R⁹ are hydrogen. In some embodiments, R⁶ is a C₁₋₆ alkyl, such as CH₃.

Non-limiting examples of a compound of formula (V) include:

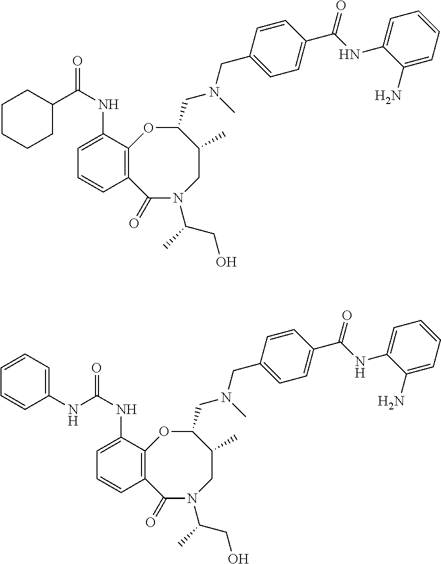 [SMILES: C[C@@H]1CN([C@@H](C)CO)C(=O)c2cccc(NC(=O)C3CCCCC3)c2O[C@@H]1CN(C)Cc1ccc(C(=O)Nc2ccccc2N)cc1 ; C[C@@H]1CN([C@@H](C)CO)C(=O)c2cccc(NC(=O)Nc3ccccc3)c2O[C@@H]1CN(C)Cc1ccc(C(=O)Nc2ccccc2N)cc1]

or a pharmaceutically acceptable salt form thereof.

Compounds of Formula (VI)

Further provided herein are compounds of formula (VI):

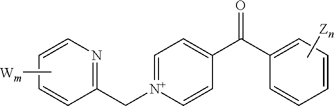

or a pharmaceutically acceptable salt form thereof,
wherein:
W and Z are independently selected from the group consisting of: halogen, OR¹, NR¹R², CN, NO₂, C₁₋₆ alkyl, C₁₋₆ alkenyl, and C₁₋₆ alkynyl;
R¹ and R² are independently selected from the group consisting of: hydrogen and C₁₋₆ alkyl;
m is an integer from 0 to 4; and
n is an integer from 0 to 5.

In some embodiments, m is 0. In some embodiments, n is 0.

A non-limiting example of a compound of formula (VI) includes:

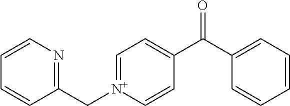 [SMILES: O=C(c1ccccc1)c1cc[n+](Cc2ccccn2)cc1]

or a pharmaceutically acceptable salt form thereof.

Compounds of Formula (VII)

In some embodiments, a compound provided herein is a compound of formula (VII):

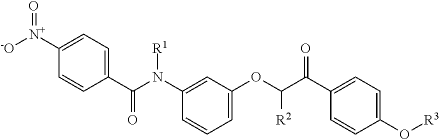

or a pharmaceutically acceptable salt form thereof,
wherein:
R¹ and R³ are independently selected from the group consisting of: hydrogen and C₁₋₆ alkyl; and
R² is selected from the group consisting of: hydrogen, C₁₋₆ alkyl, C₁₋₆ alkenyl, and C₁₋₆ alkynyl.

In some embodiments, R¹ is hydrogen. In some embodiments, R² is a C₁₋₆ alkyl. For example, R² can be CH₃. In some embodiments, R³ is a C₁₋₆ alkyl, such as CH₃.

A non-limiting example of a compound of formula (VII) includes:

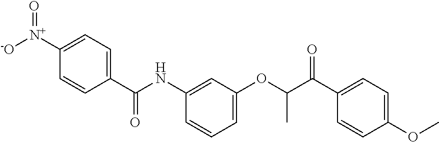

or a pharmaceutically acceptable salt form thereof.

Compounds of Formula (VIII)

Also provided herein are compounds of formula (VIII):

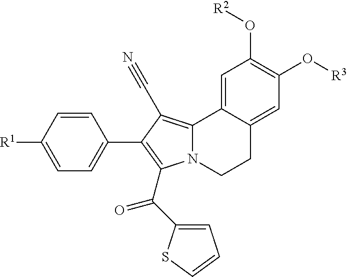

or a pharmaceutically acceptable salt form thereof,
wherein:
R¹ is selected from the group consisting of: hydrogen, C₁₋₆ alkyl, C₁₋₆ alkenyl, and C₁₋₆ alkynyl; and
R² and R³ are independently selected from the group consisting of: hydrogen and C₁₋₆ alkyl.

In some embodiments, R¹ is a C₁₋₆ alkyl. For example, R¹ can be CH₃. In some embodiments, R² and R³ are independently a C₁₋₆ alkyl. For example, R² and R³ can be CH₃.

A non-limiting example of a compound of formula (VIII) includes:

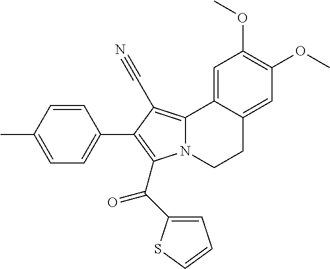

or a pharmaceutically acceptable salt form thereof.

As used herein, an “effective amount” is an amount sufficient to effect beneficial or desired results. For example, a therapeutic amount is one that achieves the desired therapeutic effect. This amount can be the same or different from a prophylactically effective amount, which is an amount necessary to delay or reduce risk of onset of disease or disease symptoms. An effective amount can be administered in one or more administrations, applications or dosages. A therapeutically effective amount of a therapeutic compound (i.e., an effective dosage) depends on the therapeutic compounds selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the 1050 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Pharmaceutical Compositions

The methods provided herein include the manufacture and use of pharmaceutical compositions, which include compounds identified by a method provided herein as active ingredients. Also included are the pharmaceutical compositions themselves.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration.

A pharmaceutical composition is typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol, or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates, or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes, or multiple dose vials made of glass or plastic.

Pharmaceutical compositions suitable for injection can include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. The composition should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The pharmaceutical compositions can also be prepared in the form of suppositories (e.g., with conventional suppository bases such as cocoa butter and other glycerides) or retention enemas for rectal delivery.

Additionally, intranasal delivery is possible, as described in, inter alia, Hamajima et al., Clin. Immunol. Immunopathol., 88(2), 205-10 (1998). Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

The pharmaceutical composition may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular patient, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

Dosage forms or compositions containing a compound as described herein in the range of 0.005% to 100% with the balance made up from non-toxic carrier may be prepared. Methods for preparation of these compositions are known to those skilled in the art. The contemplated compositions may contain 0.001%-100% active ingredient, in one embodiment 0.1-95%, in another embodiment 75-85%.

The pharmaceutical compositions can be included in a container, pack, or dispenser together with instructions for administration.

### DEFINITIONS

For the terms “for example” and “such as,” and grammatical equivalences thereof, the phrase “and without limitation” is understood to follow unless explicitly stated otherwise. As used herein, the term “about” is meant to account for variations due to experimental error. All measurements reported herein are understood to be modified by the term “about,” whether or not the term is explicitly used, unless explicitly stated otherwise. As used herein, the singular forms “a,” “an,” and “the” include plural referents unless the context clearly dictates otherwise.

A “patient,” as used herein, includes both humans and other animals, particularly mammals. Thus, the methods are applicable to both human therapy and veterinary applications. In some embodiments, the patient is a mammal, for example, a primate. In some embodiments, the patient is a human.

A “therapeutically effective” amount of a compound provided herein is typically one which is sufficient to achieve the desired effect and may vary according to the nature and severity of the disease condition, and the potency of the compound. It will be appreciated that different concentrations may be employed for prophylaxis than for treatment of an active disease.

The phrase “in combination” refers to the use of more than one therapeutic agents simultaneously or sequentially and in a manner that their respective effects are additive or synergistic.

The term “prodrug,” as used herein, refers to a compound which, upon administration to a subject, undergoes chemical conversion by metabolic or chemical processes to yield, e.g., the compounds described herein, and/or a salt and/or solvate thereof. The term “prodrugs” can include lactones for the statins provided herein. For example, simvastatin and lovastatin compounds can be administered in their inactive lactone form and are metabolized to their active hydroxy-acid forms in vivo. Such prodrugs can be administered orally since hydrolysis in many instances occurs under the influence of the digestive enzymes. Parenteral administration may also be used, e.g., in situations where hydrolysis occurs in the blood. See, e.g., Yang, D-J. and Hqang L. S., Journal of Chromatography A, 2006, 1119(1-2): 277-294; Prueksaritanont, T. et al., Drug Metab Dispos, 2002, 30(5): 505-12; and Garcia, M. J. et al., Methods Find Exp Clin Pharmacol, 2003, 25(6): 457-81.

“A pharmaceutically acceptable salt” is intended to mean a salt that retains the biological effectiveness of the free acids and bases of the specified compound and that is not biologically or otherwise undesirable. A compound provided herein may possess a sufficiently acidic, a sufficiently basic, or both functional groups, and accordingly react with any of a number of inorganic or organic bases, and inorganic and organic acids, to form a pharmaceutically acceptable salt. A person skilled in the art will know how to prepare and select suitable salt forms for example, as described in Handbook of Pharmaceutical Salts Properties, Selection, and Use By P. H. Stahl and C. G. Wermuth (Wiley-VCH 2002).

If a compound is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an α-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If a compound is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

The term, “compound,” as used herein is meant to include all stereoisomers, geometric isomers, and tautomers of the structures depicted. Compounds herein identified by name or structure as one particular tautomeric form are intended to include other tautomeric forms unless otherwise specified.

In some embodiments, a compound provided herein, or salt thereof, is substantially isolated. By “substantially isolated” is meant that the compound is at least partially or substantially separated from the environment in which it was formed or detected. Partial separation can include, for example, a composition enriched in the compound provided herein. Substantial separation can include compositions containing at least about 50%, at least about 60%, at least about 70%, at least about 80%, at least about 90%, at least about 95%, at least about 97%, or at least about 99% by weight of the compound provided herein, or salt thereof. Methods for isolating compounds and their salts are routine in the art.

The phrase “pharmaceutically acceptable” is used herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “alkyl” includes straight-chain alkyl groups (e.g., methyl, ethyl, propyl, butyl, pentyl, hexyl, heptyl, octyl, nonyl, and decyl) and branched-chain alkyl groups (isopropyl, tert-butyl, isobutyl, and sec-butyl), cycloalkyl (alicyclic) groups (cyclopropyl, cyclopentyl, cyclohexyl, cycloheptyl, and cyclooctyl), alkyl substituted cycloalkyl groups, and cycloalkyl substituted alkyl groups. In certain embodiments, a straight chain or branched chain alkyl has six or fewer carbon atoms in its backbone (e.g., C₁-C₆ for straight chain; C₃-C₆ for branched chain). The term C₁-C₆ includes alkyl groups containing 1 to 6 carbon atoms.

The term “alkenyl” includes aliphatic groups that may or may not be substituted, as described above for alkyls, containing at least one double bond and at least two carbon atoms. For example, the term “alkenyl” includes straight-chain alkenyl groups (e.g., ethylenyl, propenyl, butenyl, pentenyl, hexenyl, heptenyl, octenyl, nonenyl, and decenyl) and branched-chain alkenyl groups. The term alkenyl further includes alkenyl groups that include oxygen, nitrogen, sulfur or phosphorous atoms replacing one or more carbons of the hydrocarbon backbone. In certain embodiments, a straight chain or branched chain alkenyl group has 6 or fewer carbon atoms in its backbone (e.g., C₂₋₆ for straight chain, C₃₋₆ for branched chain). The term C₂₋₆ includes alkenyl groups containing 2 to 6 carbon atoms.

The term “alkynyl” includes unsaturated aliphatic groups analogous in length and possible substitution to the alkyls described above, but which contain at least one triple bond and two carbon atoms. For example, the term “alkynyl” includes straight-chain alkynyl groups (e.g., ethynyl, propynyl, butynyl, pentynyl, hexynyl, heptynyl, octynyl, nonynyl, and decynyl) and branched-chain alkynyl groups. The term alkynyl further includes alkynyl groups that include oxygen, nitrogen, sulfur or phosphorous atoms replacing one or more carbons of the hydrocarbon backbone. In certain embodiments, a straight chain or branched chain alkynyl group has 6 or fewer carbon atoms in its backbone (e.g., C₂₋₆ for straight chain, C₃₋₆ for branched chain). The term C₂₋₆ includes alkynyl groups containing 2 to 6 carbon atoms.

The term “carbocyclyl,” as used herein, unless otherwise indicated refers to a non-aromatic, saturated or partially saturated, monocyclic or fused, spiro or unfused bicyclic or tricyclic hydrocarbon ring referred to herein as containing a total of from 3 to 10 carbon atoms (e.g., 5-8 ring carbon atoms). Exemplary carbocyclyls include monocyclic rings having from 3-7, e.g, 3-6, carbon atoms, such as cyclopropyl, cyclobutyl, cyclopentyl, cyclohexyl, cycloheptyl and the like.

The term “aryl,” as used herein, unless otherwise indicated, includes an organic radical derived from an aromatic hydrocarbon by removal of one hydrogen, such as phenyl or naphthyl.

The term “heterocyclyl,” as used herein, unless otherwise indicated, includes a stable, mono- or multi-cyclic non-aromatic heterocyclic ring system which consists of carbon atoms and at least one heteroatom selected from the group consisting of N, O, and S, wherein the nitrogen and sulfur heteroatoms may be optionally oxidized, and the nitrogen atom may be optionally quaternized. For example, the ring can have 1, 2, 3 or 4 N, or 1, 2 or 3 O or S atoms. The heterocyclic system may be attached, unless otherwise stated, at any heteroatom or carbon atom which affords a stable structure. Examples of non-aromatic heterocycles include monocyclic groups such as: aziridine, oxirane, thiirane, azetidine, oxetane, thietane, pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin and hexamethyleneoxide. Examples of polycyclic heterocycles include: indolinyl, quinolyl, tetrahydroquinolyl, isoquinolyl, particularly 1- and 5-isoquinolyl, 1,2,3,4-tetrahydroisoquinolyl, cinnolinyl, quinoxalinyl, particularly 2- and 5-quinoxalinyl, quinazolinyl, phthalazinyl, 1,5-naphthyridinyl, 1,8-naphthyridinyl, 1,4-benzodioxanyl, dihydrocoumarin, 2,3-dihydrobenzofuryl, 1,2-benzisoxazolyl, benzothienyl, particularly 3-, 4-, 5-, 6-, and 7-benzothienyl, benzoxazolyl, benzthiazolyl, particularly 2-benzothiazolyl and 5-benzothiazolyl, purinyl, benzimidazolyl, particularly 2-benzimidazolyl, benztriazolyl, thioxanthinyl, carbazolyl, carbolinyl, acridinyl, pyrrolizidinyl, and quinolizidinyl.

The term “heteroaryl” as used herein, unless otherwise indicated, refers to a heterocycle having aromatic character. A polycyclic heteroaryl may include one or more rings which are partially saturated. Examples include tetrahydroquinoline and 2,3-dihydrobenzofuryl. Examples of heteroaryl groups include: pyridyl, pyrazinyl, pyrimidinyl, particularly 2- and 4-pyrimidinyl, pyridazinyl, thienyl, furyl, pyrrolyl, particularly 2-pyrrolyl, imidazolyl, thiazolyl, oxazolyl, pyrazolyl, particularly 3- and 5-pyrazolyl, isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

The term “halogen” includes chloro, bromo, iodo, and fluoro.

### EXAMPLES

The invention is further described in the following examples, which do not limit the scope of the invention described in the claims.

Methods.

The following methods were used in the studies described herein.

Generation of dsRed+ Leukemic Mice

Whole bone marrow were harvested from C57/B16 actin-dsRed mice (006051, Jackson Labs), subjected to red blood cell lysis (Qiagen), then granulocyte-macrophage progenitors (GMPs) were isolated by Fluorescence-activated cell sorting (FACsAria, Becton Dickinson). GMPs were spinfected twice (2500 rpm, 90 min, 37 C) in the presence of mIL3 (10 ng/ml, Peprotech), mIL6 (10 ng/ml, Peprotech), and mSCF (10 ng/ml, Peprotech) with pMSCV-MLL-AF9-Neo virus then transplanted into lethally irradiated C57/B16 recipients. After disease onset, the spleens were harvested and transplanted into sublethally irradiated secondary recipients. Subsequent transplantation of bulk spleen cells from leukemic secondary mice was repeated twice to generate leukemic GMPs from quaternary transplant leukemic mouse bone marrow.

Mouse Maintenance and Transplant Experiments

All mouse experiments were conducted with an IUCAC-approved animal protocol at respective institutions. Mice strains used in this study include C57BL/6 (Taconic), C57BL/6 actin-dsRed (Jackson Labs), B6.5JL (Taconic). Recipient mice were either sublethally (1×5.5 Gy [550 rads]) or lethally irradiated (2×5.5 Gy [550 rads]) prior to tail vein transplant. Unless otherwise noted, all transplanted cells were resuspended in 300 μl HBSS (Lonza) and loaded in 271/2 gauge syringes (309623, Becton Dickinson) for transplant.

Isolation of LSC-Enriched Murine Leukemia Cells

Long bones and hips from moribund leukemic mice were harvested, cleaned, crushed and sequentially passed through 100 and 70 μM filters (Falcon). The cells were RBC lysed (Qiagen) and stained in PBS+2% FBS (Omega). Antibodies included lineage markers CD4, CD8, Gr1, Mac1, IL-7R, and Ter119 (Caltag), c-kit (17-1172-83, eBioscience), Sca-1 (11-5981-82, eBioscience), CD34 (11-0341-85, eBioscience), and CD16/32 (25-0161-82, eBioscience). Hoechst 33258 (H21491, Invitrogen) was also used, to identify live cells. Cells were sorted on a FacsAria II (Becton Dickinson).

Isolation of Normal Murine HSPC Hematopoietic Cells

The humerii, tibiae, ilia, and femurs were isolated from actin-DsRed mice (Vintersten K, Monetti C, Gertsenstein M, et al. Genesis. 2004; 40:241-246.) that had been fully backcrossed to the C57BL/6J background. The material was cleaned, crushed and sequentially passed through 100 and 70 μM filters (Falcon). The cells were RBC lysed (ACK lysing buffer) and stained in PBS+0.5% FBS (Omega) with biotin-conjugated lineage anti-mouse antibodies CD4, CD8, CD3, B220, Gr-1, Mac-1, and Ter-119 (BD Biosciences) and the SLAM antibody CD48. The biotin-labeled cells were spun down, resuspended in 0.5% FBS in PBS, and incubated for 15-30 minutes at 4° C. with agitation with 1 mL of Dynabead M-280 streptavidin-linked magnetic beads per 4 mice. The bead-linked cells were depleted using magnetic separation and rinsed once. The lineage- and CD48-depleted cell fraction was labeled with streptavidin-APC-Cy7 antibody from BD Biosciences and c-kit-APC, Sca-1-FITC, CD48-Pacific Blue, and CD150 PE-Cy7 antibodies from eBioscience. DsRed-positive lineage-Sca-1+c-kit+CD48− HSCs were sorted using a FACS DiVa or FACS ARIA (BD Biosciences).

Isolation and Maintenance of Primary GFP+Mesenchymal Stem Cells (MSCs)

The humerii, tibiae, ilia, femurs, and spine were isolated from actin-GFP mice (000329, Jackson Lab) crushed using a mortar and pestle, washed in phosphate-buffered saline (PBS) without magnesium and calcium (Gibco) with 0.5% fetal bovine serum (FBS) (HyClone) and filtered through a 70-μm filter. Red blood cell lysis was performed with ammonium-chloride/potassium-chloride (ACK) lysing buffer (Lonza), the cells were resuspended in α-MEM (StemCell Technologies), 20% FBS (HyClone) and 1× Pen-Strep (CellGro), plated in 25 ml in 150-cm² tissue-culture flasks (three per mouse), and incubated at 33° C. with 5% CO₂. After 2-3 days, the medium was replaced with fresh α-MEM with 20% FBS. After 8-14 days, the cells were rinsed, split by trypsinization (CellGro), pooled, filtered through a 70 μm filter, replated at 3-4 million cells per 150-cm² tissue-culture flask, and grown at 33° C. with 5% CO₂ for 3-4 days until nearly confluent. The cells were trypsinized, filtered, and resuspended in 0.5% FBS in PBS with biotin-conjugated anti-mouse CD105 antibody (eBioscience) for 15-30 minutes at room temperature. The CD105-labeled cells were incubated with Dynabead M-280 streptavidin-linked magnetic beads (Invitrogen) for 15-30 minutes at 4° C. with agitation and isolated using magnetic separation and rinsed once. The CD105⁺ cell fraction was replated at 1-2 million cells per 150-cm² tissue-culture flask and incubated at 33° C. with 5% CO₂ for 2-3 days.

The MSCs were trypsinized, filtered, diluted in phenol-red-free alpha-MEM with 20% FBS, and plated (30 μl for a total of 2000 cells per well) on 384-well clear-bottomed black tissue-culture treated plate (3712, Corning) pretreated with fibronectin (Millipore). Plate covers (VWR) were added, the plates were spun and at 500 with slow braking, and the cells were incubated at room temperature for 60-90 minutes before incubation at 33° C. with 5% CO₂ for 3 days prior to hematopoietic cell plating.

HSPC Primary Screen Protocol

A total of 20 μL of 20 μg/mL fibronectin (Millipore) in PBS was added to each well of a 384-well clear-bottomed black tissue-culture treated plate (Corning) and incubated for 30-120 minutes at 33° C. or 37° C. During this time the BMSC were trypsinized, filtered, and diluted to 66,700 cells/mL in phenol-red-free alpha-MEM with 20% FBS. The fibronectin solution was removed from each well using a 24-channel wand aspirator; then 30 μL of BMSC solution was added to plate 2000 BMSCs/well. Liquid addition was either made using a multichannel pipettor or liquid-dispensing system, which were determined to be equivalent in terms of reproducibility. Each plate was covered with a sterile rayon breathable membrane (VWR) and plastic lid, and then spun at 500 rpm (approximately 60× gravity) with slow braking. The plates were incubated at room temperature for 60-90 minutes before incubation at 33° C. with 5% CO2 for 3 days prior to HSC addition.

The sorted HSCs, SLAMs and progenitor cells were resuspended in phenol-red free alpha-MEM with 20% FBS and diluted to 10,000 cells/mL. A total of 20 μL of cell solution containing 200 hematopoietic cells was added to each well using a multichannel pipettor or liquid-dispensing system. Each plate was covered with a sterile rayon breathable membrane (VWR) and plastic lid, and then spun at 500 rpm (approximately 60×gravity) with slow braking. The plates were incubated at room temperature for 60-90 minutes before incubation at 33° C. with 5% CO2 overnight prior to compound or cytokine addition.

Co-cultured cells in 384-well plates were imaged using a TexasRed filter centered at 559 nM and GFP filter centered at 469 nM at 40-x total magnification using the ImageXpress Micro from Molecular Devices Corporation (MDC). Image analysis was performed using MetaXpress software from MDC and CellProfiler software from the Broad Institute of Harvard and MIT.

Compound addition and incubation was performed as described for the LSC coculture screen above, but without a media change or readdition of the compound.

For the final IC50 experiment with HSCs and MSCs, where a total of 400 HSCs were plated in each well, there were a total of six replicates and the LSC positive control, XK469, (Sigma-Aldrich) was used for comparison.

Cell Culture

OP9 stromal cells (ATCC) transduced with a GFP+ lentiviral construct by standard procedures were cultured in α-MEM (36453, Stem Cell Technologies) with Sodium Bicarbonate (25080-094, Gibco), L-glutamine (071000, StemCell Technologies), β-mercaptoethanol (ES-007-E, Chemicon International), 20% FBS, and 1× Pen-Strep. For optimal stromal support of primary leukemia cells, these cells were not employed until several weeks post thaw and serial batches were tested in 384-well format for supportiveness before use. Where cultured briefly in vitro in suspension, primary murine leukemia cells were plated in IMDM (12440053, Invitrogen) with 10% FBS, 10 ng/ml mIL3, and 1× Pen-Strep.

Statistics

Kaplan Meyer analysis was done using Prism 5 (GraphPad) software. All other statistical analysis was done with R (www.r-project.org) or Excel (Microsoft) software. Curve fitting was performed by standard procedures using Cook's distance with MatLab software. Averages were calculated as means unless otherwise noted and error bars represent standard error of the means.

Leukemia Coculture Assay Screening Protocol

Using an automated liquid dispenser (Multidrop Combi, 5840300, Thermo Scientific), 10 μl of 0.1% gelatin (ES006B, Chemicon International) was added to each well of a black, barcoded, clear-bottom 384 well plate (3712, Corning) and incubated at room temperature for 15 minutes. After a wash step, 6,750 OP9 cells were added in 50 μl of OP9 media (500 ml α-MEM (36453, Stem Cell Technologies) with 14.6 ml Sodium Bicarbonate (25080-094, Gibco), 5 ml L-glutamine (071000, StemCell Technologies), 2.5 ml Beta-mercaptoethanol (ES-007-E, Chemicon International), 20% FBS (10082-147, Gibco), and 1% Pen-Strep (15140-122, Gibco)) to each well of the gelatin-coated plates, plate covers (B90112, VWR) were added, and the plates were placed in the incubator for 24 hours. The plate covers were removed, the media was aspirated from each well using a Microplate washer (ELx405, BioTek), and 300 flow-sorted leukemia cells were added in 50 μl of 50% conditioned OP9 media (3 days) and 50% Coculture media (500 ml DMEM (11965-092, Gibco), 10% Horse Serum (26050-088, Gibco), 1:100 Hydrocortisone (07904, StemCell Technologies), 2.5 ml Beta-mercaptoethanol (ES-007-s E, Chemicon International), 10% FBS (10082-147, Gibco) and 1% Pen-Strep (15140-122, Gibco)) to each well. Plate covers were re-added and the plates were placed in the incubator for 24 hours. The plate covers were removed, 100 nl of compound in dimethyl sulfoxide (DMSO) (final concentration 5 μM in 0.2% DMSO) was added to the appropriate wells using a CyBi-Well Vario (CyBio) the plate covers were replaced, and the plates were placed in the incubator for 3 days. Plate covers were removed, the media was aspirated from each plate, 50 μl of fresh media (50/50 coculture and OP9 conditioned media mix) was added and another 100 nl of compound was added to the appropriate wells. The plates were re-covered and placed in the incubator for 2 days. At this point, the plates were imaged using a IX Microscope (Molecular Devices) at 10× magnification at 9 sites per well in both the GFP and dsRed channels. Images were stored on the Broad Institute server for future analysis.

Identification of Primary Coculture Screen Hits

To choose compounds for further retesting, a two-point normalization between mean of DMSO (set at zero) and positive control cytotoxic XK469 (set at −100% DMSO kill) was performed within each plate. Each experimental compound was thus represented as a percent effect within this normalized range. The lowest (in magnitude) % inhibition required to achieve statistical significance in both replicates on any one plate within a screening run was identified. This cutoff was then used to permissively identify hits across all plates that achieved this degree of inhibition, regardless of whether a z-score of less than −3 was observed.

Stromal Toxicity Screen

OP9 or MSCs were plated in white, 384 well plates (3570, Corning) and 24 hours later compounds were added in 8-point dose as described. Three days later the plates were put out to cool to room temperature, the cultures were aspirated, and 50 μl of CellTiterGlo reagent (G7570, Promega) diluted 1:3 in PBS was added to each well. The plates were covered and placed on an orbital shaker for 20 minutes then analyzed using either an LJL Analyst (LJL) or Envision (Perkin Elmer). For each compound the concentration at which each compound resulted in statistically significant killing of MSCs or OP9s was determined. The compounds were ranked and only those that exhibited toxicity at ≧10 uM were retained for further selectivity testing.

AML Cell Line Screen

Nomo-1, THP-1, SKM-1, NB4, and U937 cell lines were grown in RPMI (12-702F, BioWhittaker), 10% FBS (10082-147, Gibco), and 1% Pen-Strep (15140-122, Gibco) and OCI-AML3 was grown in α-MEM (36453, Stem Cell Technologies), 10% FBS (10082-147, Gibco), and 1% Pen-Strep (15140-122, Gibco). 3000 cells were plated in each well in 30 μl in white, 384 well plates (3570, Corning) and 16 hours later 100 nl of the appropriate compound was added to each well. 72 hours later the plates were cooled to room temperature and 30 μl of CellTiterGlo reagent (G7570, Promega) diluted 1:1 in PBS was added to each well and the plates were analyzed using an Envision (Perkin Elmer).

Stromal Pretreatment Screen

OP9 cells were plated as in the primary screen. 24 hours later compounds were added to the stromal cultures and the plates were incubated for three days. The wells were aspirated and washed twice with PBS after which flow-sorted leukemia cells were plated as described. 3 days later the media was changed and 2 days after that the plates were imaged and analyzed as in the primary screen. Importantly, potency did not correlate with presence of an effect.

In the context of primary MSC stroma (uncolored), both HSPCs (from actin-GFP mice (000329, Jackson Lab)) and LSC (dsRed+) populations were comingled together in a ratio of 2:1 within the same 384format wells. As in the stromal pretreatment screen with OP9 stroma, BRD7116 was added for three days to the stroma prior to the addition of the hematopoietic populations. 5 days after compound addition, wells were imaged in the red and green channels and total cells were counted using MetaXpress software from MDC.

RNA Isolation, Gene Expression Profiling, and Data Analysis

To elucidate potential cell-autonomous effects of BRD7116, primary leukemia cells were exposed to either 5 μM BRD7116 or DMSO vehicle for 6 hours in suspension in IMDM (12440053, Invitrogen) with 10% FBS, 10 ng/ml mIL3, and 1× Pen-Strep.

RNA was isolated using a trizol-chloroform protocol or with a Qiagen RNeasy kit (74104, Qiagen). Total RNA from the samples was normalized to 20 ng/μl and the Illumina® TotalPrep™-96 RNA Amplification Kit (Applied Biosystems, PN #4393543) protocol was used for amplification in a semi automated process. The total RNA underwent reverse transcription to synthesize first-strand cDNA. This cDNA was then converted into a double-stranded DNA template for transcription. In vitro transcription synthesized aRNA and incorporated a biotin-conjugated nucleotide. The aRNA was then purified to remove unincorporated NTPs, salts, enzymes, and inorganic phosphate. Labeled cRNA was normalized to 150 ng/μl and hybridized to Illumina's Illumina's MouseRef-8 v2.0 Expression BeadChip. The labeled RNA strand was hybridized to the bead on the BeadChip containing the complementary gene-specific sequence After a 16 hour hybridization, the beadchips were washed and stained using a Cy3 streptavidin conjugate. Illumina's BeadArray Reader was used to measure the fluorescence intensity at each addressed bead location.

Gene-expression profiles were generated by using mouse ref-8 DNA microarray (Illumina) according to manufacturer's instruction. Raw data were normalized by cubic spline method implemented in Illumina Normalizer module of GenePattern analysis tool kit (www.broadinstitute.org/genepattern), and converted into human gene symbols based on the orthologous gene mapping table provided by Jackson laboratory. For each compound, a ranked list of genes was created by comparing the treated samples to DMSO control samples. The genes were ordered using the signal-to-noise statistic (the difference of means in each group scaled by the sum of standard deviations computed over 3 treatment replicates). The resulting data was analyzed using Gene Set Enrichment Analysis method (Subramanian, A., Tamayo, P., Mootha, V. K., Mukherjee, S., Ebert, B. L., Gillette, M. A., Paulovich, A., Pomeroy, S. L., Golub, T. R., Lander, E. S., et al. (2005). Proc Natl Acad Sci USA 102, 15545-15550.).

Coculture Pulse Treatment Experiment

13,500 GFP+ OP9 cells were plated onto gelatin coated, 96 well black, clear bottom plates (3904, Corning). 24 hours later, 2,000 sorted leukemia cells were plated onto the monolayer. Compounds (5 μM) were added either 1 (early pulse) or 4 (late pulse) days later at the same 5 μM working concentration of main coculture screen. In both cases, compounds were removed after 24 hours of treatment, the wells were washed, and fresh media without compound was added. The plates were imaged and analyzed 6 days after leukemia cell plating. The viability of the stroma was assessed using stromal monolayers grown alone and treated in parallel, with the same readout described in the stromal toxicity screen above.

Annexin V Apoptosis Assay

1.5 million primary leukemia cells were plated in 96-well plates in IMDM with 10% FBS and 10 ng/ml mIL3 (Peprotech). Compounds were added to a final DMSO concentration of 0.02%. 24 hours later the cells were harvested, spun down, resuspended in PBS with 2% FBS and stained with Annexin V antibody (88-8007-72, eBioscience) for 30 minutes. The cells were analyzed by flow cytometry on a FacsCanto II (Becton Dickinson).

In Vivo Pooled shRNA screen

Viral packaging protocols known in the art were used for the arrayed virus for subsequent pools. Briefly, 100 ng of lentiviral plasmid, 100 ng of packaging plasmid (psPAX2) and 10 ng of envelope plasmid (VSV-G) were used to transfect packaging cells (293T) with TransIT-LT1 (Minis Bio). Virus was harvested 48 and 70 hours post-transfection. The two harvests were combined and assessed for titer. Viruses targeting enzymes in the HMG-CoA pathway were generated. These arrayed viruses were then combined at equal titers to generate a pool of shRNA-lentiviruses.

Flow sorted L-GMPs were resuspended to 5M per ml in IMDM, 10% FBS, 10 ng/ml mIL-3 (Peprotech), 10 ng/ml mIL-6 (Peprotech), 20 ng/ml mSCF (Peprotech), and 5 μg/ml polybrene (Sigma Aldrich). 400 μl of cell material and 400 μl of pooled virus was added to 5 wells of a 12 well plate. The cells were spun at 2500 rpm, 37° C., for 90 minutes. Two hours later an additional 1.25 ml (primary screen) or 800 μl (additional screens) of fresh IMDM, 10% FBS, 10 ng/ml mIL-3, 10 ng/ml mIL-6, 20 ng/ml mSCF was added to each well and the plates were put in the incubator for 24 hours. After incubation each well was split with half the cells frozen for processing (see below) and the other half transplanted into 5 sublethally irradiated recipients. After 2 weeks, the recipient mice were sacrificed and the bone marrow was harvested for processing.

Harvested cells were resuspended in 1 ml PBS and lysed according to the QIAamp Blood Mini kit (Qiagen). The hairpin region was PCR amplified from the purified gDNA using the following conditions. 5 μL primary PCR primer mix, 4 μL dNTP mix, 1×Ex Taq buffer, 0.75 μL of Ex TaqDNA polymerase (TaKaRa), and 6 μg genomic DNA in a total reaction volume of 50 pt. Thermal cycler PCR conditions consisted of heating samples to 95° C. for 5 min; 15 cycles of 94° C. for 30 sec, 65° C. for 30 sec, and 72° C. for 20 sec; and 72° C. for 5 min. PCR reactions were them pooled per sample. A secondary PCR step was performed containing 5 μM of common barcoded 3′ primer, 8 μL dNTP mix, 1×Ex Taq buffer, 1.5 μL Ex TaqDNA polymerase, and 30 μL of the primary PCR mix for a total volume of 90 μL. 10 μL of independent 5′ barcoded primers are then added into each reaction, after which the 100 μL total volume is divided into two 50 μL final reactions. Thermal cycler conditions for secondary PCR are as follows: 95° C. for 5 min; 15 cycles of 94° C. for 30 sec, 58° C. for 30 sec, and 72° C. for 20 sec; and 72° C. for 5 min. Individual 50 μL reactions are then re-pooled. Reactions are then run on a 2% agarose gel and intensity-normalized. Equal amounts of samples are then mixed and gel-purified using a 2% agarose gel. Samples were sequence using a custom sequencing primer using standard Illumina conditions.

The raw sequencing data was normalized independently for each replicate. The raw read counts for each shRNA were normalized to the total reads and the calculated fold change of normalized reads between two time points was divided by the mean fold change of all the control shRNAs over the same time points. A gene was considered a hit if two shRNAs had greater than a fold change of 10.

Alamar Blue Assay for Human Progenitor Cell Toxicity

Normal G-CSF mobilized CD34+ cells (AllCells, LLC, Emeryville, Calif.), normal BM CD34 cells (MSKCC, New York, N.Y.) and cord blood CD34+ cells (New York Blood Center, New York) were previously frozen and thawed out before the proliferation assay was set up in 384-well plates. Briefly, 50 μl of IMDM medium containing 20% FBS (Gemini Bio-Product, West Sacramento, Calif.), 20 ng/ml of recombinant human Kit Ligand (rhKL), 20 ng/ml of rh-Interleukin-3 (rhIL-3), 20 ng/ml of rhG-CSF, 6 units/ml of rhEPO, 10-42-mercaptoethanol, 2 mM glutamine, 50 u/ml penicillin, 50 μg/ml streptomycin, 1,000 viable CD34+ cells in the presence of various doses of Broad compounds (drug-treated group) or 0.1% DMSO (control group) were incubated at 37° C. After 6 days, 5 μl of AlamaBlue (AbD Serotec, Raleigh, N.C.) was added to each well and further incubated overnight. The fluorescence signal was then measured with GeminiXS microplate reader (Molecular Devices, Sunnyvale, Calif.). Effect of small molecules to proliferation of CD34+ cells was reported as Relative fluorescent intensity (Relative Fluorescent Intensity=Fluorescent Intensity (drug-treated)/Fluorescent Intensity (Control).

Human CD34⁺ CAFC Assays

Primary samples were obtained from peripheral blood and/or bone marrow of consented AML patients during initial presentation or relapse. Samples were centrifuged over Ficoll-Paque PLUS (GE Heathcare) step gradient (2000×g for 30 min in 50 ml tubes) to obtain mononuclear cells and then CD34⁺ cells were selected with immunomagnetic beads (MACS Cell Separation, Miltenyi Biotec, Auburn, Calif.), except in one case, NPM1 mutation, which was CD34⁻ by flow cytometry and mononuclear cells were used. 50,000 cells were placed into 12.5 cm² flasks (Becton Dickinson Labware, Franklin Lakes, N.J.) in co-culture with the MS-5 mouse bone marrow-derived stromal cell line (kindly provided by Dr. Itoh, Dept. of Biology, Niigata University, Japan) and DMSO control and the number of final cobblestones was later read. The number of cells needed to form one cobblestone was then placed in triplicate for each sample with each drug at serial drug dilutions from 10 μM to 0.63 μM as well as with DMSO 0.5% for control purposes and incubated overnight with Iscove's modified Dulbecco's medium (IMDM, in house media preparation laboratory)+20% fetal calf serum (FCS, Atlanta Biologicals, Lawrenceville, Ga.) with glutamine (2 mM/ml, in house media preparation laboratory), monothioglycerol (MTG, 10 nM/ml, Sigma Cell Culture, St. Louis Mo.), c-Kit ligand (20 ng/ml, Amgen, Thousand Oaks, Calif.), Flt ligand (20 ng/ml, Imclone, Bridgewater, N.J.), TPO (20 ng/ml, Amgen) and IL-3 (20 ng/ml, Amgen). Cells were then washed to remove the drugs and placed into 96 well cell culture plates (Becton Dickinson) with α-MEM (in house media preparation laboratory)+12.5% FCS+12.5% horse serum (Gemini Bio-products, West Sacramento, Calif.) with glutamine, MTG, hydrocortisone (1 nM/ml, Sigma) and IL-3 in co-culture with MS-5 stromal cells. After five weeks (except for the FLT3-ITD sample, for which two weeks has been shown to be final), the wells were read as positive or negative for cobblestone formation. The same procedure was followed with normal CD34+ cells obtained from cord blood.

Automated Image Analysis

Image analysis was performed using CellProfiler software (Carpenter et al., Genome Biology, 7(10):R100 (2006); Carpenter et al., Proc Nat Acad Sci USA 106(6):1826-1831 (2009)) (Broad Institute, Inc). An image analysis pipeline, or a serial set of image analysis algorithms, was constructed to measure dozens of features in the dsRed-labeled foreground objects and GFP-labeled stromal cell layer. Each of nine sites per well was analyzed independently, and the image processing was parallelized by sending small batches of images to the Broad Institute's computing cluster. The analyzed data was merged and stored in a MySQL (Oracle, Inc.) database.

Each site was processed as follows. First, the well boundary (if present) was identified and masked within the image. Illumination correction was performed to correct for persistent illumination variations across each image (due to many possible sources, including optical hardware irregularities, illumination patterns, or shading). Illumination functions were created by smoothing raw each channel independently with a large median filter (350×350 pixels), respecting the well boundary. Each channel's raw image is then divided by its respective illumination function before subsequent processing. Next, dsRed objects were segmented by thresholding at 1.3 times the mode of the image intensity histogram. To identify the center peaks of objects, especially dim, low-contrast cobblestone-like objects, we employed a Laplacian of Gaussian (LoG) morphological operator with a Gaussian width of 7 pixels. These LoG centers were masked by the dsRed-positive regions described above, to exclude spurious well-edge artifacts. The filtered LoG objects were morphologically expanded to segment individual dsRed objects, and multiple measurements were performed on these objects (including intensity, area and shape, object neighbors, and texture). In addition, GFP stromal coverage was measured using a threshold of 1.2 times the image intensity histogram mode, and this was used as a metric of stromal survival.

The above per-object measurements were used in a supervised machine learning method to distinguish cobblestone objects from differentiated cells. Gentle Boosting classifiers were trained and iterative feedback was used to refine the high-dimensional decision boundary between the two dsRed phenotypes. When satisfied with the classification, every object in every image was scored as either cobblestone or differentiated with the set of rules returned from the classifier. The cobblestone objects were nearly impossible to segment with a high degree of certainty, even by close visual inspection, so cobblestone area per well was used as a suitable proxy for cobblestone cell count, and was the primary readout of the assay.

Syngeneic Transplantation of Comingled HSPCs and LSCs in Coculture

Heterotypic cocultures containing dsRed+ LSCs, CD45.1+ HSPCs, and GFP+ MSCs were exposed to compounds for 48 hours, then transplanted en masse post trypsinization with untreated wild-type helper splenocytes (CD45.1+CD45.2+) into lethally irradiated, wildtype recipient animals (CD45.2+). Latency of leukemia onset was compared for mice receiving cocultures treated with compound compared to DMSO control treated cocultures. The engraftment of the normal HSPCs treated and injected along with the leukemia cells was quantified by FACs analysis of the bone marrow of mice alive at the 16 week endpoint across treatments.

Mevalonolactone Rescue Experiment

Primary leukemia cocultures were plated as described, and treated with 2 mM mevalanolactone and/or 1 μM lovastatin as shown, for 5 days. Total leukemia cells were counted using MetaXpress software from MDC. Carriers were controlled for across all treatments. The viability of the stroma was assessed using stromal monolayers grown alone and treated in parallel, with the same readout described in the stromal toxicity screen here.

### Example 1

### An Ex Vivo System to Probe Primary Leukemia Cells in the Context of Microenvironmental Support

A systematic exploration of leukemia stem cell (LSC) sensitivities aids in the development of novel therapeutics that favorably impact long-term clinical outcomes. Described herein is a method to identify small molecules that selectively target LSCs.

Generation of Murine AML Model and Isolation of Stem Cell Enriched Leukemia Fraction

In order to consistently generate sufficient quantities of primary LSCs for screening, the well-characterized MLL-AF9 retroviral murine model of acute myeloid leukemia (AML) (Krivtsov, A. V., Twomey, D., Feng, Z., Stubbs, M. C., Wang, Y., Faber, J., Levine, J. E., Wang, J., Hahn, W. C., Gilliland, D. G., et al. (2006). Nature 442, 818-822.) generated in a 13-actin-dsRed transgenic background (Vintersten, K., Monetti, C., Gertsenstein, M., Zhang, P., Laszlo, L., Biechele, S., and Nagy, A. (2004). Genesis 40, 241-246.) was employed, allowing for rapid identification of leukemia cells (dsRed⁺) within heterotypic cell cultures. MLL-AF9 transforms non-self-renewing granulocyte-monocyte progenitors (GMPs) into an aggressive myelomonocytic leukemia, and, in this murine model, the functionally-defined LSCs display a defined immunophenotype shared with normal GMPs (Lin^{lo}, Sca-1⁻, c-kit⁺, FcYRII^{hi}, CD34^{hi}) (Krivtsov, A. V., Twomey, D., Feng, Z., Stubbs, M. C., Wang, Y., Faber, J., Levine, J. E., Wang, J., Hahn, W. C., Gilliland, D. G., et al. (2006). Nature 442, 818-822. In the primary transplant of this model the reported frequency of LSCs ranges from 1 in 6 in the GMP gate by flow cytometry analysis (Krivtsov, A. V., Twomey, D., Feng, Z., Stubbs, M. C., Wang, Y., Faber, J., Levine, J. E., Wang, J., Hahn, W. C., Gilliland, D. G., et al. (2006). Nature 442, 818-822.) to as few as 1 in 4 myeloid cells carrying the translocation (Somervaille, T. C., and Cleary, M. L. (2006). Cancer Cell 10, 257-268.). MLL translocations occur in over 70% of infant leukemias (Biondi, A., Cimino, G., Pieters, R., and Pui, C. H. (2000). Blood 96, 24-33.) and 10% of leukemias overall (Huret, J. L., Dessen, P., and Bernheim, A. (2001). Leukemia: official journal of the Leukemia Society of America, Leukemia Research Fund, UK 15, 987-989.) and are associated with aggressive disease (Pajuelo-Gamez, J. C., Cervera, J., Garcia-Casado, Z., Mena-Duran, A. V., Valencia, A., Barragan, E., Such, E., Bolufer, P., and Sanz, M. A. (2007). Cancer Genet Cytogenet 174, 127-131.). To generate leukemia with a short and predictable latency while further enriching for stem cell activity, the primary transplant leukemia cells were serially transplanted through secondary, tertiary, and quaternary murine recipients. Briefly, Granulocyte-Monocyte Progenitors (GMPs) were sorted from β-actin dsRed mice, transduced with retrovirus carrying the MLL-AF9 oncogenic fusion gene, and transplanted into lethally-irradiated wild type recipient mice. At disease onset, splenocytes were subsequently harvested and transplanted through 3 additional rounds of recipient animals to generate quaternary leukemic mice. Whole bone marrow was harvested from quaternary animals at disease onset, and the LSC-enriched population was isolated by fluorescence activated cell sorting (FACS) using predefined immunophenotypic markers (dsRed⁺ c-kit^{hi} CD16/32^{hi} CD34^{hi}). All of the dsRed⁺ leukemia cells in the quaternary transplants were Lin^{lo} and that nearly all of the dsRed⁺ c-kit^{hi} cells fell into the GMP (FcYRII^{hi} CD34^{hi}) gate. All primary leukemia cells used for secondary experiments were therefore isolated by flow cytometry using these cell surface markers, whereas full GMP gating (Krivtsov, A. V., Twomey, D., Feng, Z., Stubbs, M. C., Wang, Y., Faber, J., Levine, J. E., Wang, J., Hahn, W. C., Gilliland, D. G., et al. (2006). Nature 442, 818-822.) was used during assay development, primary screening and retest screening using the top 5-10% of c-kit^{hi} cells.

Maintaining Primary Leukemia Cells In Vitro Using Heterotypic Coculture

As with other primary cell populations, the LSCs derived from this murine model do not grow well in isolation in vitro and required cytokine support for short term suspension culture (Langdon, S. P. (2004). (Totowa, N.J., Humana Press). Krivtsov, A. V., Twomey, D., Feng, Z., Stubbs, M. C., Wang, Y., Faber, J., Levine, J. E., Wang, J., Hahn, W. C., Gilliland, D. G., et al. (2006). Nature 442, 818-822.). Therefore a system was sought for growing these cells in vitro in a way that allowed the monitoring of a marker associated with self-renewal while maintaining disease re-initiation potential, defining properties of leukemia stem cells. It was hypothesized that an ex vivo recapitulation of the in vivo bone marrow niche would allow these challenges to be addressed, and would allow both cell-autonomous and non-cell-autonomous mechanisms of LSC support to be probed. Historically, the in vitro monitoring of normal HSPC frequency has been best achieved by coculturing primary bone marrow (or stem enriched) cells on supportive stromal monolayers, and using either cobblestone formation in the cobblestone area forming culture (CAFC) assay or colony formation in the LTC-IC assay as a readout of stem cell activity (Breems, D. A., Blokland, E. A., Neben, S., and Ploemacher, R. E. (1994). Leukemia 8, 1095-1104.; Ploemacher, R. E., van der Sluijs, J. P., van Beurden, C. A., Baert, M. R., and Chan, P. L. (1991). Blood 78, 2527-2533.; Bock, T. A. (1997). Stem Cells 15 Suppl 1, 185-195.). Leukemia stem cell studies have also made use of these assays (Terpstra, W., Ploemacher, R. E., Prins, A., van Lom, K., Pouwels, K., Wognum, A. W., Wagemaker, G., Lowenberg, B., and Wielenga, J. J. (1996a). Blood 88, 1944-1950.; Terpstra, W., Prins, A., Ploemacher, R. E., Wognum, B. W., Wagemaker, G., Lowenberg, B., and Wielenga, J. J. (1996b). Blood 87, 2187-2194.). A number of supportive stromal cell populations have been described, including the mouse bone marrow derived OP9 cell line that can maintain HSCs in culture for many weeks (Nakano, T., Kodama, H., and Honjo, T. (1994). Generation of lymphohematopoietic cells from embryonic stem cells in culture. Science 265, 1098-1101.; Roecklein, B. A., and Torok-Storb, B. (1995). Functionally distinct human marrow stromal cell lines immortalized by transduction with the human papilloma virus E6/E7 genes. Blood 85, 997-1005.; Mendez-Ferrer, S., Michurina, T. V., Ferraro, F., Mazloom, A. R., Macarthur, B. D., Lira, S. A., Scadden, D. T., Ma'ayan, A., Enikolopov, G. N., and Frenette, P. S. (2010). Nature 466, 829-834.). Thus the primary leukemia cells were cultured on bone marrow stroma to support the LSCs in vitro and enable the monitoring of the cobblestone cellular morphology associated with self-renewal in the CAFC assay, at high throughput. Bone marrow stromal cell populations expressing green fluorescent protein (GFP) were generated, allowing for the rapid identification and analysis of both the leukemia cells (dsRed') and the stroma (GFP⁺). Two populations of bone marrow-derived murine cells were employed-primary mesenchymal stem cells (MSCs) from actin-GFP mice (Schaefer, B. C., Schaefer, M. L., Kappler, J. W., Marrack, P., and Kedl, R. M. (2001). Cell Immunol 214, 110-122), and GFP-expressing OP9 cells.

The primary leukemia cells had a number of interesting features when cocultured on OP9 or primary MSC monolayers. First, the leukemia cells grew robustly on both types of stroma in the absence of cytokine supplementation (FIGS. 1A and 1B). Interestingly, the leukemia cells did not appear to be randomly distributed across the stroma. Rather, some leukemia cells grew under a subset of cells in the stromal monolayer, forming morphologically distinct cellular aggregates reminiscent of cobblestones in the classic CAFC assay. Moreover, cell culture media that had been conditioned on stromal cells for 3 days increased the frequency of cobblestoned leukemia cells, reflecting the supportive nature of secreted stromal factors (FIG. 1H). Consistent with the utility of cobblestoning as an in vitro marker of LSC health and self-renewal, c-kit^{hi} leukemia cells formed dramatically more cobblestones than c-kit^{lo} leukemia cells obtained from the same sick mouse, consistent with the known enrichment of LSCs in the c-kit^{hi} population (FIG. 1B) (Krivtsov, A. V., Twomey, D., Feng, Z., Stubbs, M. C., Wang, Y., Faber, J., Levine, J. E., Wang, J., Hahn, W. C., Gilliland, D. G., et al. (2006). Nature 442, 818-822.). Leukemia cells cultured on stroma for 4 weeks were also able to re-initiate disease in lethally irradiated mouse recipients (FIG. 1I), reflecting a preservation of LSC function under these coculturing conditions.

An Imaging Analysis Algorithm to Quantify Cobblestone Formation in Coculture at Scale

While leukemic cells could generally be distinguished from stromal cells in coculture, by virtue of the dsRed and GFP channels, manual identification and quantification of cobblestones was not feasible at high throughput scale. Therefore the development of an automated image analysis algorithm able to recognize the subtle morphology of cells within a cobblestone was required (Carpenter, A. E., Jones, T. R., Lamprecht, M. R., Clarke, C., Kang, I. H., Friman, O., Guertin, D. A., Chang, J. H., Lindquist, R. A., Moffat, J., et al. (2006). Genome Biol 7, R100.). The CellProfiler software used as a platform is freely available on the internet at cellprofiler.org and is described in (Carpenter, A. E., Jones, T. R., Lamprecht, M. R., Clarke, C., Kang, I. H., Friman, O., Guertin, D. A., Chang, J. H., Lindquist, R. A., Moffat, J., et al. (2006). Genome Biol 7, R100.; Jones, T. R., Carpenter, A. E., Lamprecht, M. R., Moffat, J., Silver, S. J., Grenier, J. K., Castoreno, A. B., Eggert, U.S., Root, D. E., Golland, P., et al. (2009). Proc Natl Acad Sci USA 106, 1826-1831.)

Examples of cobblestoned and non-cobblestoned cells (captured in the dsRed wavelength) were used to train a machine-learning algorithm to utilize image-based measurements of the cells to recognize and quantify the total levels of cobblestoned cell area per image. The software defined 50 ‘rules’ based on a variety of features, including shape, intensity, texture, and cell neighbor relationships that delineated cobblestoned cells from non-cobblestoned cells (FIG. 1C). The rules were identified from a larger set of possible rules, and the most important rules could vary from experiment to experiment, as they were automatically selected by the program.

In one set of experiments, the most important rules were as shown in FIG. 8.

An exemplary set of 50 rules follows:

IF (CellsGFP_Texture_GaborY_CorrGFP_3 > 12.5014, [0.79889772144625182,
−0.79889772144625182], [−0.58185660421140362, 0.58185660421140362])
IF (CellsGFP_Neighbors_PercentTouching_2 > 46.153799999999997,
[−0.30067284091552265, 0.30067284091552265], [1.0, −1.0])
IF (CellsGFP_Intensity_StdIntensity_CorrGFP > 0.022568000000000001,
[0.82336373685976005, −0.82336373685976005], [−0.24309844747124817,
0.24309844747124817])
IF (CellsGFP_Intensity_MinIntensity_CorrGFP > 0.077369400000000005,
[− 0.4728142633216722, 0.4728142633216722], [0.6235737415380187,
−0.6235737415380187])
IF (CellsGFP_Neighbors_NumberOfNeighbors_2 > 2.0,
[−0.37709022810074727, 0.37709022810074727], [0.66150956054333987,
−0.66150956054333987])
IF (CellsGFP_Texture_DifferenceEntropy_CorrGFP_3 > 1.8080499999999999,
[0.85839068549325914, −0.85839068549325914], [−0.21779020163125526,
0.21779020163125526])
IF (CellsGFP_Zernike_4_0 > 0.051085600000000002, [0.74393701366150489,
−0.74393701366150489], [−0.33049000031214143, 0.33049000031214143])
IF (CellsGFP_Texture_GaborX_CorrGFP_3 > 10.3916, [0.4716635009171678,
−0.4716635009171678], [−0.52963951747162075, 0.52963951747162075])
IF (CellsGFP_Texture_InverseDifferenceMoment_CorrGFP_3 > 0.199321,
[−0.27407376258219901, 0.27407376258219901], [0.83557559199430187,
−0.83557559199430187])
IF (CellsGFP_Neighbors_FirstClosestYVector_2 > 5.2201000000000004,
[0.98361867671572201, −0.98361867671572201], [−0.15791207397379678,
0.15791207397379678])
IF (CellsGFP_Intensity_StdIntensity_CorrGFP > 0.022568000000000001,
[0.67363456612513362, −0.67363456612513362], [−0.19809389250680284,
0.19809389250680284])
IF (CellsGFP_Texture_SumAverage_CorrGFP_1 > 9.3404299999999996,
[−0.33785820043175446, 0.33785820043175446], [0.63529161362004238,
−0.63529161362004238])
IF (CellsGFP_Texture_Variance_CorrGFP_1 > 6.6745599999999996,
[−0.98455546384131687, 0.98455546384131687], [0.2252504369321246,
−0.2252504369321246])
IF (CellsGFP_Neighbors_PercentTouching_2 > 46.153799999999997,
[−0.21134074543733844, 0.21134074543733844], [1.0, −1.0])
IF (CellsGFP_Intensity_StdIntensity_CorrGFP > 0.023512700000000001,
[0.75778341587667009, −0.75778341587667009], [−0.19639457576010935,
0.19639457576010935])
IF (CellsGFP_Zernike_7_7 > 0.017016300000000002, [0.34729403205146309,
−0.34729403205146309], [−0.68147841561087541, 0.68147841561087541])
IF (CellsGFP_Zernike_3_1 > 0.135487, [0.59362724228569397,
−0.59362724228569397], [−0.35003360423953461, 0.35003360423953461])
IF (CellsGFP_Location_Center_X > 615.82600000000002,
[0.63245520542616951, −0.63245520542616951], [−0.33104046636425605,
0.33104046636425605])
IF (CellsGFP_Intensity_StdIntensity_CorrGFP > 0.022568000000000001,
[0.77154312068397524, −0.77154312068397524], [−0.24156341189882483,
0.24156341189882483])
IF (CellsGFP_Neighbors_SecondClosestXVector_2 > −5.9005700000000001,
[−0.16351692276327784, 0.16351692276327784], [0.91068329991958963,
−0.91068329991958963])
IF (CellsGFP_Texture_InverseDifferenceMoment_CorrGFP_3 >
0.23797499999999999, [−0.26620186041244748, 0.26620186041244748],
[0.60053150551975265, −0.60053150551975265])
IF (CellsGFP_Neighbors_PercentTouching_2 > 74.1935,
[−0.32737190533715838, 0.32737190533715838], [0.57865450431561294,
−0.57865450431561294])
IF (CellsGFP_Neighbors_SecondClosestObjectNumber_2 > 457.0,
[−0.37283567125228462, 0.37283567125228462], [0.54274712417741344,
−0.54274712417741344])
IF (CellsGFP_Location_Center_X > 613.17100000000005,
[0.59210947783983714, −0.59210947783983714], [−0.31092592917583661,
0.31092592917583661])
IF (CellsGFP_Texture_GaborY_CorrGFP_3 > 3.4533700000000001,
[0.17931758887426547, −0.17931758887426547], [−1.0, 1.0])
IF (CellsGFP_Zernike_6_0 > 0.13461500000000001, [0.6762308966820314,
−0.6762308966820314], [−0.27484522125494837, 0.27484522125494837])
IF (CellsGFP_Intensity_StdIntensity_CorrGFP > 0.022568000000000001,
[0.73806096273961785, −0.73806096273961785], [−0.27993435719751664,
0.27993435719751664])
IF (CellsGFP_Zernike_8_0 > 0.0072755399999999996, [0.23236285809834142,
−0.23236285809834142], [−0.82618077340261853, 0.82618077340261853])
IF (CellsGFP_Texture_SumEntropy_CorrGFP_3 > 2.0000900000000001,
[0.63645202890693542, −0.63645202890693542], [−0.33356491049875309,
0.33356491049875309])
IF (CellsGFP_Texture_SumAverage_CorrGFP_3 > 9.875,
[−0.47380303659303985, 0.47380303659303985], [0.40024972429313754,
−0.40024972429313754])
IF (CellsGFP_Texture_DifferenceEntropy_CorrGFP_3 > 1.8080499999999999,
[0.93503856954238485, −0.93503856954238485], [−0.19941722947039434,
0.19941722947039434])
IF (CellsGFP_Intensity_MeanIntensityEdge_CorrGFP >
0.093252500000000002, [−0.43752812363058691, 0.43752812363058691],
[0.42301342711812201, −0.42301342711812201])
IF (CellsGFP_Texture_GaborY_CorrGFP_3 > 9.9892299999999992,
[0.44905110121765107, −0.44905110121765107], [−0.40825680170421735,
0.40825680170421735])
IF (CellsGFP_Texture_SumVariance_CorrGFP_1 > 7.50413,
[−0.31913015096215785, 0.31913015096215785], [0.74686062194612168,
−0.74686062194612168])
IF (CellsGFP_AreaShape_Solidity > 0.86956500000000003,
[0.19921146328582284, −0.19921146328582284], [−0.91574708628408719,
0.91574708628408719])
IF (CellsGFP_Texture_GaborY_CorrGFP_3 > 12.5014, [0.54603748183180034,
−0.54603748183180034], [−0.31435279930349264, 0.31435279930349264])
IF (CellsGFP_Texture_DifferenceVariance_CorrGFP_3 > 0.25,
[−0.27414328750902306, 0.27414328750902306], [0.84917541698746135,
−0.84917541698746135])
IF (CellsGFP_Zernike_5_1 > 0.023795, [−0.19050013983911987,
0.19050013983911987], [0.78407440703416453, −0.78407440703416453])
IF (CellsGFP_Texture_InfoMeas2_CorrGFP_1 > 0.88842600000000005,
[−0.28296920589845131, 0.28296920589845131], [0.52525943069440173,
−0.52525943069440173])
IF (CellsGFP_Intensity_StdIntensity_CorrGFP > 0.023512700000000001,
[0.78939216000591017, −0.78939216000591017], [−0.21383384577458389,
0.21383384577458389])
IF (CellsGFP_Neighbors_FirstClosestYVector_2 > 2.9338600000000001,
[0.69515017327666628, −0.69515017327666628], [−0.27172807446565267,
0.27172807446565267])
IF (CellsGFP_Texture_SumAverage_CorrGFP_1 > 8.5999999999999996,
[−0.20201660528251084, 0.20201660528251084], [0.73859696790148355,
−0.73859696790148355])
IF (CellsGFP_Texture_GaborY_CorrGFP_3 > 9.9892299999999992,
[0.39688375157078121, −0.39688375157078121], [−0.50471551369836298,
0.50471551369836298])
IF (CellsGFP_Zernike_4_0 > 0.048531299999999999, [0.70324723055261407,
−0.70324723055261407] , [−0.37101216618504573, 0.37101216618504573])
IF (CellsGFP_Texture_Variance_CorrGFP_1 > 6.6745599999999996,
[−0.99889885214737162, 0.99889885214737162], [0.2148031828208545,
−0.2148031828208545])
IF (CellsGFP_Intensity_StdIntensity_CorrGFP > 0.023512700000000001,
[0.76846550314448991, −0.76846550314448991], [−0.29187603552275571,
0.29187603552275571])
IF (CellsGFP_Neighbors_PercentTouching_2 > 46.153799999999997,
[−0.15799954526209009, 0.15799954526209009], [1.0, −1.0])
IF (CellsGFP_Texture_InverseDifferenceMoment_CorrGFP_3 >
0.23797499999999999, [−0.23454806781627119, 0.23454806781627119],
[0.65290241056027143, −0.65290241056027143])
IF (CellsGFP_AreaShape_FormFactor > 0.86067800000000005,
[0.37983435274178351, −0.37983435274178351], [−0.51014599300620067,
0.51014599300620067])
IF (CellsGFP_Neighbors_FirstClosestYVector_2 > −3.46373,
[0.19616214527928263, −0.19616214527928263], [−0.66642319457830657,
0.66642319457830657])

Adaptation of the Coculture System to a 384-Well High Throughput Format

High throughput screening required adaptation of the heterotypic culture system to a 384-well plate format. Numerous assay parameters were optimized, most notably employing gelatin pre-coating of wells to prevent stromal monolayer peeling, optimizing the number of stromal cells plated per well while minimizing the time spent in suspension at plating, attaching porous plate covers to prevent irregular evaporation, and including media pre-conditioned by OP9 cells at LSC plating. Automated liquid handling equipment and high throughput microscopy were also employed, allowing the imaging of 384-well plates in both the dsRed and GFP channels. Ultimately, the 384-well coculturing system demonstrated a sensitivity of 85%, with a z-prime factor of 0.27, yielding a system suitable for high-throughput screening of heterotypic cultures.

### Example 2

### A High-Throughput Small Molecule Screen to Identify Mediators of Leukemia Biology within the Niche

Having defined a platform to examine complex, primary leukemia cell biology within the context of the bone marrow microenvironment, a small molecule screen was performed to identify LSC sensitivities otherwise inaccessible by traditional cell-based assays and biochemical, target-based screens.

Primary Screening Identifies Compounds that Inhibit Leukemic Cobblestoning in Coculture

A primary screen was performed in duplicate in 384-well plates with 14,720 compounds selected from a series of commercially available and proprietary libraries (see Table A). Two of the libraries included compounds generated via diversity oriented synthesis (DOS) (Schreiber, S. L. (2000). Science 287, 1964-1969.).

Briefly, primary dsRed⁺ leukemia cells enriched for LSCs were isolated by flow cytometry and cocultured on GFP⁺ OP9 stromal monolayers, treated with 5 μM of compound (to a final concentration of 0.2% DMSO), and imaged 5 days later by automated microscopy (dsRed channel for leukemia, and GFP channel for stroma) (FIG. 1). The stored images were analyzed for levels of cobblestoning as described herein. All data was normalized to both negative control wells containing 0.2% DMSO and to positive control wells containing XK469, a topoisomerase II inhibitor (Gao, H., Huang, K. C., Yamasaki, E. F., Chan, K. K., Chohan, L., and Snapka, R. M. (1999). Proc Natl Acad Sci USA 96, 12168-12173.). 415 compounds were identified that decreased leukemic cobblestoned morphology by at least 3 standard deviations from the negative control in both replicates, with essentially no overlap observed between the positive and negative control wells (FIG. 1D).

TABLE A
Composition of the libraries used in the primary screen of
14,720 compounds.

| Library Plate | Compounds | Description |
|---|---|---|
| Bioactives | 1920 | Commercial libraries; known |
| | | biological activity |
| Chromatin Biased | 1920 | Broad Institute; Chromatin modifying |
| (DOS) | | (e.g. HDAC) enzyme inhibitors |
| Commercial A | 2240 | Commercial libraries (TimTec, |
| | | Maybridge and ChemDiv) |
| Kinase Biased | 6080 | Computationally selected for ATP |
| | | binding site interactors |
| Natural Products | 1600 | Natural products with fully charac- |
| | | terized structure |
| Stereo Diverse | 960 | Broad Institute; multiple stereo- |
| (DOS) | | chemistries represented |

Counterscreening Further Defines Potent, Leukemia Selective Compounds

Three counterscreens were executed to exclude compounds that inhibited normal HSPCs in coculture, to prioritize compounds with the most potent and reproducible dose-dependent anti-leukemic activity, and to exclude compounds that scored as hits merely by causing direct stromal toxicity.

First, data generated in a related parallel screen of normal dsRed⁺ HSPCs (Lin^{lo} Sca-1⁺ c-Kit⁺ CD48^{low}, from β-actin-dsRed mice) grown on primary GFP⁺ MSCs was examined. These cocultures were exposed to 20 μM of compounds within the same libraries of compound for 5 days, then total dsRed+ cell numbers were quantified using an automated image-based readout. Notably, HSPCs cocultured in this fashion and then transplanted into mice are able to engraft and reconstitute all lineages in recipient animals at 16 weeks. By this cross-comparison, hits from the leukemia primary screen that were found to reproducibly decrease normal HSPC growth by at least 80% compared to DMSO controls were discarded, as were hits showing overt toxicity to the stromal cells (assessed by visual inspection in the GFP channel). Note that this filtering served as a first, coarse filter of non-selective cytotoxics.

Next, to identify compounds that reproducibly and potently inhibited leukemic cobblestoning in a dose-dependent manner, a retest of the remaining 254 compounds was performed on the leukemia cocultures using OP9 stroma (8-point dose) and using primary MSCs (4-point dose). 60% of the compounds had an IC₅₀ at or below 5 μM on both types of stroma (considered a positive retest), on both types of stroma, confirming the robustness and reproducibility of the assay (FIG. 1E). Additionally, the potency of the compounds in the retest correlated with the degree of activity in the primary screen. 196 compounds that exhibited activity on either stromal cell type were carried forward.

Finally, to exclude compounds that inhibited leukemia cell cobblestoning by directly killing the stroma, the remaining 196 compounds were added in 8-point dose to OP9 and MSC stromal monolayers in the absence of leukemia cells. Under the same assay conditions as in the original screen, the viability of each well was determined by CellTiter-Glo analysis at the assay endpoint. 36 compounds displayed stromal toxicity at multiple doses (FIG. 1F) and were removed.

### Example 3

### Secondary Screens Identify Leukemia-Selective Compounds with Distinct Activity Profiles

The assay described herein was then used to identify two classes of leukemia-selective compounds within the 160: those that would not have been hits in traditional cell line screens and those that likely inhibit leukemia cobblestoning by modifying the biology of the niche. These compounds may highlight new opportunities for biological and therapeutic investigation beyond what traditional screening approaches reveal. These compounds were identified using three secondary screens: a traditional human AML cell line screen, a stromal pretreatment screen in which only the stromal cells were exposed to compound, prior to the addition of leukemia cells, and additional LSC and HSPC coculture retesting for dose curve refinement of selectivity.

First, a traditional small molecule screen was performed on six human AML cell lines (U937, THP-1, NOMO-1, SKM-1, NB4, and OCI-AML3), two of which (NOMO-1, THP-1) contain the same oncogene (MLL-AF9) used to generate the primary leukemia cells utilized in our coculture system. The cell lines were grown in isolation under standard conditions, treated with the 160 compounds at 8-point dose, and three days later the viability of each well was quantified using CellTiter-Glo reagent. The IC50s from these AML cell line screens were then compared to the IC50s from the coculture screens. A set of compounds were identified that demonstrated at least 10-fold more potent on primary leukemia cells in coculture compared to the average potency observed across the AML cell lines (FIG. 2D). As discussed, the existence of such differentially-active compounds is consistent with our hypothesis that coculturing can expand the pool of therapeutically promising compounds identified in high throughput format. Importantly, a lack of activity against cell lines does not discount the therapeutic potential of given hits from the screening system, as the biologically complex system in the present assays may be more predictive of therapeutic relevance. A set of compounds were also identified that were 10-fold more potent on the cell lines, decreasing the likelihood that primary cells are simply more sensitive (FIG. 2D).

Next, having noted in the primary screen that leukemia inhibition is sometimes accompanied by stromal morphological changes, suggesting that effects on the niche might contribute to this inhibition (FIG. 2A), a stromal pretreatment screen was performed. The 160 compounds were added to OP9 stromal monolayers in 8-point dose for 3 days, after which the stromal layers were washed thoroughly, and the primary leukemia cells were added (FIG. 2B). As before, the plates were imaged after 6 days of coculture, and the level of cobblestoning was quantified. Any inhibition of cobblestoning observed under these modified assay conditions would presumably be due to the activity of the compound on the niche, and would allow the analysis of cell non-cell-autonomous mechanisms of leukemia growth inhibition.

Troglitazone, a peroxisome proliferator-activated receptor-γ (PPAR-γ) agonist previously approved for the treatment of diabetes (Knowler, W. C., Hamman, R. F., Edelstein, S. L., Barrett-Connor, E., Ehrmann, D. A., Walker, E. A., Fowler, S. E., Nathan, D. M., and Kahn, S. E. (2005). Diabetes 54, 1150-1156.; Memon, R. A., Tecott, L. H., Nonogaki, K., Beigneux, A., Moser, A. H., Grunfeld, C., and Feingold, K. R. (2000). Endocrinology 141, 4021-4031.), illustrated the utility of this stromal pretreatment secondary screen. This compound inhibited leukemia cobblestoning in the stromal pretreatment screen yielding a dose response curve very similar to that from the coculture retest screen in which both leukemia and stroma were exposed to compound (FIG. 2C). This decrease in leukemic cobblestoning was accompanied by what appeared to be a dose-dependent adipocytic change in the stroma. Consistent with this observation, PPAR-γ agonists are known to induce adipocytic change (Gimble, J. M., Robinson, C. E., Wu, X., Kelly, K. A., Rodriguez, B. R., Kliewer, S. A., Lehmann, J. M., and Morris, D. C. (1996). MolPharmacol 50, 1087-1094.), OP9 stromal cells can readily differentiate into adipocytes (Wolins, N. E., Quaynor, B. K., Skinner, J. R., Tzekov, A., Park, C., Choi, K., and Bickel, P. E. (2006). J Lipid Res 47, 450-460.), and adipocytes are known to antagonize hematopoietic cell self-renewal (Naveiras, O., Nardi, V., Wenzel, P. L., Hauschka, P. V., Fahey, F., and Daley, G. Q. (2009). Nature 460, 259-263.). In contrast, sensitivity to troglitazone was not observed in any of the cell lines (FIG. 2C). Additional compounds were identified that appeared to act via stromal modification, consistent with the hypothesis that the present complex screening system described herein identifies leukemia cell dependencies that could not be explored in the absence of stromal coculture.

Finally, the LSC selectivity of the identified compounds relative to normal HSPCs was more rigorously characterized by additional coculture retesting. Given the limited HSPC cell number in wildtype mouse bone marrow, a subset of compounds selected for novelty, biological interest, and therapeutic potential was more rigorously examined. Structural analogues of some of these high-interest compounds were also obtained. The selected set was screened in 8-point dose against both normal HSPCs with a high number of replicates (6) and LSC-enriched leukemia cells, each cocultured on primary MSCs. Confirming the previous selection criteria, these compounds were differentially active against leukemia cells compared to HSPCs, with some exhibiting more than a 100-fold leukemia selectivity (Table 1). Importantly, small molecules already known to have preferential activity against LSCs compared to normal HSPCs were identified in the assay, underscoring the biological robustness and relevance of this multidimensional screening approach. For example, parthenolide, a sesquiterpene lactone reported to selectively kill LSCs (Guzman, M. L., Rossi, R. M., Karnischky, L., Li, X., Peterson, D. R., Howard, D. S., and Jordan, C. T. (2005). Blood 105, 4163-4169.), was a hit in the screening system, as was celastrol, a molecule identified by gene expression analysis to act via a parthenolide-like mechanism (Hassane, D. C., Guzman, M. L., Corbett, C., Li, X., Abboud, R., Young, F., Liesveld, J. L., Carroll, M., and Jordan, C. T. (2008). Blood 111, 5654-5662.). 2-methoxy-estradiol, a microtubule inhibitor known to lack myelosuppressive side effects (Escuin, D., Burke, P. A., McMahon-Tobin, G., Hembrough, T., Wang, Y., Alcaraz, A. A., Leandro-Garcia, L. J., Rodriguez-Antona, C., Snyder, J. P., Lavallee, T. M., et al. (2009). Cell Cycle 8, 3914-3924.), also displayed leukemia selectivity. Additionally, AMD3100, an antagonist of SDF-1/CXCR4 signaling between hematopoietic and stromal cells that increases leukemia sensitivity to chemotherapy (Nervi, B., Ramirez, P., Rettig, M. P., Uy, G. L., Holt, M. S., Ritchey, J. K., Prior, J. L., Piwnica-Worms, D., Bridger, G., Ley, T. J., et al. (2009). Blood 113, 6206-6214.), while not examined by way of screening, showed selectivity for LSCs over HSPCs when tested (FIG. 2E), further reflecting the ability of our assay to probe relevant stromal sensitivities.

By combining the results from these secondary screens, classes of leukemia-selective compounds with distinct activity profiles were identified. Some compounds, such as two benzimidazole carbonates, parbendazole and methiazole, independently demonstrated potent, selective activity against primary leukemia in coculture and also showed potent activity in the AML cell line screens (IC50s<0.625 μM across 6 cell lines, Table 1 and FIGS. 2F-2G). Another set of compounds potently killed primary leukemia cells in coculture without having pronounced effects on the leukemia cell lines, while others acted by modifying the biology of the niche.

Two compounds illustrative of these latter two groups, BRD7116 and lovastatic acid, were chosen to further explore the nature of what the screening system described herein can find beyond traditional approaches.

TABLE 1
Prioritized Screening Hits Display LSC Selectivity
Compared to HSPCs under Identical Coculture Conditions

| Prioritized | | Leukemia IC₅₀ | Normal IC₅₀ |
|---|---|---|---|
| Hit | Structure | (nM) | (nM) |
| Parthenolide | 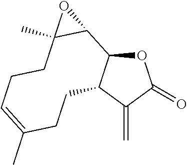 [SMILES: C=C1C(=O)O[C@@H]2[C@H]3O[C@]3(C)CC/C=C(/C)CC[C@@H]12] | 7,500 | >20,000 |
| Celastrol | 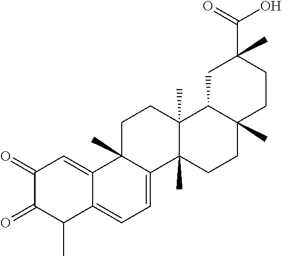 [SMILES: CC1C(=O)C(=O)C=C2C1=CC=C1[C@@]2(C)CC[C@@]2(C)[C@@H]3C[C@](C)(C(=O)O)CC[C@]3(C)CC[C@]12C] | 102 | >20,000 |
| Piperlongumine | 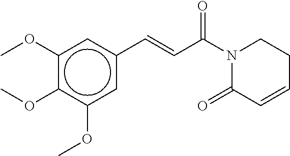 | 379 | ~20,000 |
| 2-Methoxy estradiol | 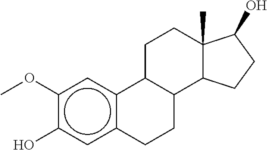 | 77 | >20,000 |
| BRD7116 | 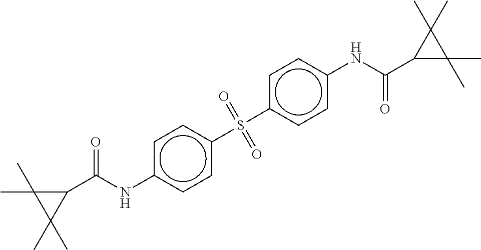 | 204 | >20,000 |
| Lovastatic acid | 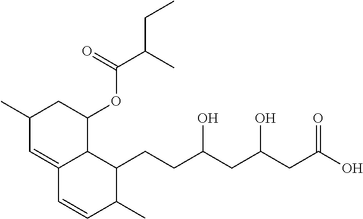 [SMILES: CCC(C)C(=O)OC1CC(C)C=C2C=CC(C)C(CCC(O)CC(O)CC(=O)O)C21] | 167 | >20,000 |
| Parbendazole | 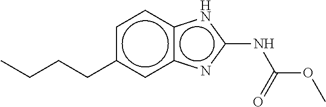 | 76 | >20,000 |
| Methiazole | 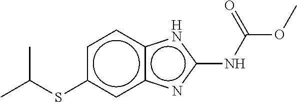 | 466 | >20,000 |
| BRD1686 | 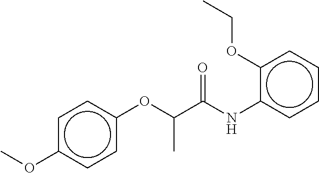 | 1,420 | >20,000 |
| BRD9608 | 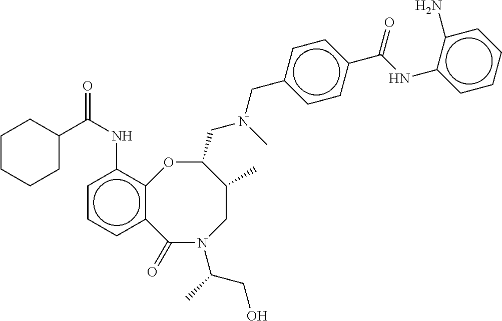 | 699 | ~20,000 |
| BRD6708 | 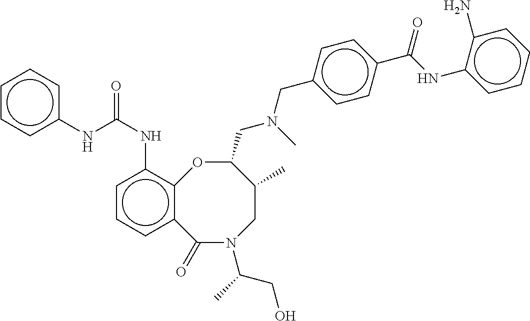 | 2,139 | >20,000 |
| BRD1319 | 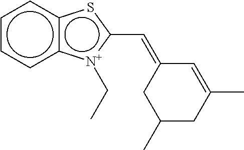 | ≦10 | >20,000 |
| BRD0638 | 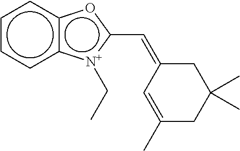 | ≦10 | >20,000 |
| BRD1856 | 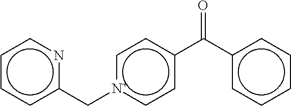 | 813 | >20,000 |
| BRD6491 | 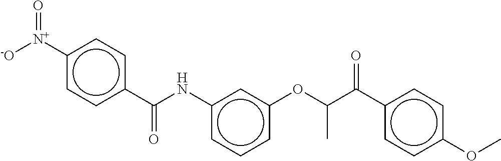 | 236 | >20,000 |
| BRD8404 | 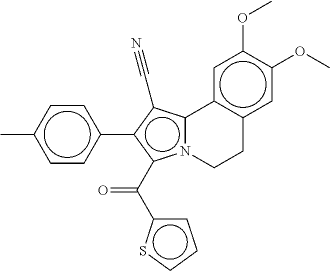 | 149 | >20,000 |

The 16 most robust and conceptually interesting small molecule hits are shown with chemical structures. Four of these compounds have been previously established as leukemia-selective (Parthenolide, Celastrol, Piperlongumine, 2-Methoxy-Estradiol), and thus serve to validate the screening approach.
The IC₅₀ values (8-point dose) for each compound, against both LSCs and HSPCs cultured under identical conditions on primary MSC stroma, are also shown.

### Example 4

### A Bis-Arylsulfone, BRD7116, Modifies the Stromal Niche and Induces Myeloid Differentiation

Compounds that act by altering the biology of the niche may uncover new leukemia selective dependencies that are non-cell-autonomous. One such compound identified, BRD7116, is a bis-arylsulfone (FIG. 3A). BRD7116 was only weakly active against AML cell lines (roughly 50% inhibition relative to DMSO control) at concentrations greater than 30 μM (FIG. 3F), and normal, primary HSPCs in coculture were not inhibited even at 20 μM, the maximum dose tested (FIG. 3B). In contrast, the IC₅₀ for the inhibition of primary leukemia in coculture was 204 nM (FIG. 3B). Furthermore, pretreatment of the stroma alone partially recapitulated the leukemia cobblestone inhibition observed when both the stroma and leukemia cells were treated (FIG. 3C). This niche-based effect was not merely a result of drug-induced stromal toxicity, as confirmed by additional stromal viability testing and stromal morphology assessments. Importantly, the observed anti-leukemia effect of stromal pretreatment likely underestimated the potency of the compound on the niche for two reasons. First, compound was only present for three days prior to LSC plating and then removed for the subsequent 6 days of coculture in order to not directly expose the LSCs. Second, media conditioned by untreated OP9 cells was added after stromal drug exposure to support the leukemia cells as described, potentially restoring stromally secreted factors antagonized by the pretreatment.

This experiment was also performed in the context of primary MSC stroma. Both HSPCs and LSC populations were comingled together in the same wells, as a “triple” coculture. As in the stromal pretreatment screen with OP9 stroma, when BRD7116 was added for three days to the stroma prior to the addition of the hematopoietic populations, an inhibition of the leukemia cells was observed (FIG. 3D). In contrast, a decrease in the numbers of comingled HSPCs was not observed, consistent with a non-cell-autonomous mechanism of leukemia inhibition that is selective.

To elucidate potential cell-autonomous effects of this compound, primary leukemia cells were exposed to either 5 μM BRD7116 or DMSO vehicle for 6 hours in suspension, harvested, and processed for gene expression analysis. Comparison of the BRD7116 and DMSO expression profiles using gene set enrichment analysis (GSEA) (Subramanian, A., Tamayo, P., Mootha, V. K., Mukherjee, S., Ebert, B. L., Gillette, M. A., Paulovich, A., Pomeroy, S. L., Golub, T. R., Lander, E. S., et al. (2005). Proc Natl Acad Sci USA 102, 15545-15550.; Mootha, V. K., Lindgren, C. M., Eriksson, K. F., Subramanian, A., Sihag, S., Lehar, J., Puigserver, P., Carlsson, E., Ridderstrale, M., Laurila, E., et al. (2003). Nat Genet. 34, 267-273.) revealed the induction of an AML differentiation program (as seen with addition of all-trans retinoic acid (ATRA) to ATRA-sensitive human AML cells) (Park, D. J., Vuong, P. T., de Vos, S., Douer, D., and Koeffler, H. P. (2003). Blood 102, 3727-3736.) in leukemia cells treated with BRD7116 (FIG. 3E). This result was consistent with the observed decrease of an in vitro marker associated with self-renewal, leukemic cobblestoning, as self-renewal is lost when stem cells undergo differentiation. Compared to DMSO control, a mild induction of apoptosis was also observed in primary leukemia cells exposed to BRD7116 in suspension for 22 hours (FIG. 3G).

In summary, BRD7116, a bis-arylsulfone, selectively inhibits leukemic cobblestoning in both a cell-autonomous and non-cell-autonomous fashion.

### Example 5

### Lovastatic Acid is a Leukemia Stem Cell Selective Agent not Revealed by Traditional Cell Line Screening

Another compound lacking a pronounced efficacy in traditional cell line screens, but showing potent, selective activity against primary leukemia cobblestoning within the stromal niche, was Lovastatic acid (FIG. 4A). This compound was one of the most differentially toxic compounds found. Lovastatic acid inhibited the primary cocultured leukemia cells with an IC₅₀ of less than 200 nM (FIG. 4B) compared to an IC₅₀ of greater than 10,000 nM across the AML cell lines (FIG. 4B), and showed minimal toxicity against normal HSPCs in coculture (FIG. 4B). Furthermore, when the treatment of the primary leukemia cells in coculture was shortened to 24 hours, and administered either at Day 1 or Day 4 post LSC plating, a strong inhibition of leukemia cells remained apparent at the Day 6 assay endpoint (FIG. 4C). Consistent with these results, compared to DMSO control, leukemia cells exposed to lovastatic acid for 22 hours in suspension underwent apoptosis as assessed by annexin V staining (FIG. 4A).

Lovastatic acid is the activated derivative of lovastatin, an FDA-approved statin in widespread clinical use for hypercholesterolemia. Statins inhibit HMG-CoA reductase (HMGCR), the enzyme catalyzing the rate-limiting step of cholesterol biosynthesis. Consistent with this mechanism, we found that addition of mevalonolactone, the metabolite immediately downstream of HMGCR, rescued the anti-leukemia effects of lovastatic acid in coculture (FIG. 4D). Further confirming this mechanism, 5 additional statins (simvastatin, fluvastatin, cerivastatin, rosuvistatin, atorvastatin) selectively inhibited leukemia cobblestoning in coculture compared to normal HSPCs (Table B).

TABLE B
IC₅₀ values for statins on LSCs and HSPCs cocultured on
primary MSC stroma

| | Leukemia IC₅₀ | Normal IC₅₀ |
|---|---|---|
| Statin (acid form) | (nM) | (nM) |
| Lovastatin | <200 | >10,000 |
| Cerivastatin | <10 | >20,000 |
| Simvastatin | ≦15 | >20,000 |
| Fluvastatin | 27.9 | >20,000 |
| Rosuvistatin | 1,222 | >20,000 |
| Atorvastatin | ≦1,900 | >20,000 |

To confirm the in vivo importance of this mechanism within the context of a true hematopoietic niche and to dissect the pathway further, a pooled in vivo short hairpin RNA (shRNA) interference screen was performed. In 5 replicates, primary, murine leukemia cells were transduced with a lentiviral shRNA pool consisting of shRNAs targeting 9 genes central to the mevalonate metabolism pathway (at least 5 shRNAs per gene) and 7 control shRNAs that do not target any murine gene. After 24 hours, half of the cells were harvested and half were transplanted into sublethally irradiated mice. After two weeks, the bone marrow and spleen of the resulting leukemic mice were harvested. Massively parallel sequencing of genomic DNA was used to determine the representation of each shRNA in leukemia cells at the time of injection (aka at 24 hours) and at 2 weeks in vivo. As previously described, genes for which multiple shRNAs comparatively deplete are required for leukemia growth in vivo (Bric, A., Miething, C., Bialucha, C. U., Scuoppo, C., Zender, L., Krasnitz, A., Xuan, Z., Zuber, J., Wigler, M., Hicks, J., et al. (2009). Cancer Cell 16, 324-335.; Meacham, C. E., Ho, E. E., Dubrovsky, E., Gertler, F. B., and Hemann, M. T. (2009). Nat Genet. 41, 1133-1137.). Consistent with the in vitro findings, multiple shRNAs targeting the Hmgcr gene were powerfully depleted at 2 weeks (FIG. 4E).

Importantly, the results of this in vivo shRNA screen serve not only to confirm the essentiality of HMGCR in an AML leukemia model, but also address the physiological relevance of our ex vivo assay approach. That the same mechanistic dependency was essential to leukemia both in a genetic screen within the bona fide bone marrow niche in vivo, and in a small molecule screen within a simulated niche ex vivo, serves to further validate the screening system described herein.

In addition to Hmgcr, multiple shRNAs targeting Farnesyltransferase (Fnta) and Isoprenylcysteine Carboxyl Methyltransferase (Icmt), enzymes further downstream in the mevalonate pathway, were also significantly depleted in vivo. As these genes code for proteins that assemble and stabilize, respectively, prenylation modifications on target proteins, the pooled screen results highlight the potential importance of protein prenylation as a key functional consequence of HMGCR inhibition in LSCs. Experiments confirmed that various farnesylation transferase inhibitors (FTIs) and geranylgeranyl transferase inhibitors (GGTIs), compounds that antagonize protein prenylation downstream of mevalonate, also inhibited leukemia cobblestoning in our coculture system (FIG. 4G). Moreover, L-744, 832, an FTI (Kohl, N. E., Omer, C. A., Conner, M. W., Anthony, N.J., Davide, J. P., deSolms, S. J., Giuliani, E. A., Gomez, R. P., Graham, S. L., Hamilton, K., et al. (1995). Nat Med 1, 792-797.), independently scored in our primary screen and passed initial selectivity filtering steps (FIG. 4H).

### Example 6

### Triple Cocultures and Syngeneic Transplantation Toward Further Validation of BRD7116 and Lovastatic Acid Selectivity

To further validate the leukemia-selective effects of these compounds, heterotypic cultures consisting of three primary cell populations were treated. dsRed⁺ LSCs and GFP⁺ HSPCs (from Ubiquitin C-GFP mice) were plated onto uncolored primary MSCs, allowing for image based analysis of normal and leukemic hematopoietic cells under admixed conditions. As in the original assay approach, treatment of these triple cocultures began one day after hematopoietic cell plating and was assessed 5 days later. Compared to DMSO control, exposure to 200 nM of lovastatic acid or 1 μM of BRD7116 selectively cleared the dsRed⁺ leukemic cells from the mixed cultures while the normal HSPCs continued to display healthy, cobblestoned morphologies (FIG. 5A).

As in vivo readouts may better reflect hematopoietic stem cell function compared to in vitro readouts (Purton, L. E., and Scadden, D. T. (2007). Cell Stem Cell 1, 263-270.; Bock, T. A. (1997). Stem Cells 15 Suppl 1, 185-195.), our results were confirmed using murine transplantation studies. Assays have been described in which primary cells enriched for LSCs are treated with compounds in suspension cultures, then transplanted into recipient mice for in vivo testing of disease re-initiation, a defining property of stem cells (Guzman, M. L., Rossi, R. M., Karnischky, L., Li, X., Peterson, D. R., Howard, D. S., and Jordan, C. T. (2005). Blood 105, 4163-4169.; Nguyen, L. V., Vanner, R., Dirks, P., and Eaves, C. J. (2012). Nat Rev Cancer 12, 133-143.). Compounds that increase disease latency relative to DMSO control are considered active. The present experiments were designed to examine not only the in vivo functional effects of treatment on cocultured leukemia stem cells, but also on normal HSPCs treated alongside the leukemia cells under identical conditions. Bone marrow repopulating ability is a functional measure of normal HSPCs, assessed by quantifying long-term engraftment and differentiation patterns in recipient mice (Bock, T. A. (1997). Stem Cells 15 Suppl 1, 185-195.; Nguyen, L. V., Vanner, R., Dirks, P., and Eaves, C. J. (2012). Nat Rev Cancer 12, 133-143.). To this end, heterotypic cocultures containing dsRed⁺ LSCs, CD45.1⁺ HSPCs, and GFP⁺ MSCs were exposed to compounds for 48 hours, then transplanted en masse post trypsinization with untreated wild-type helper splenocytes (CD45.1⁺ CD45.2⁺) into lethally irradiated, wildtype recipient animals (CD45.2⁺). Compared to DMSO control, a compound that selectively impaired leukemia cell growth by brief coculture treatment should result in both prolonged survival (i.e. extended latency of leukemia onset) of recipient mice and high levels of HSPC engraftment.

Treatment with BRD7116 resulted in a mild, but statistically significant prolonged latency of leukemia onset (FIG. 5B). Strikingly, mice that received lovastatic acid treated mixed cultures survived substantially longer than the DMSO control group (FIG. 5C). Furthermore, the engraftment of the normal HSPCs treated and injected along with the leukemia cells was not impaired, as evidenced by equivalent numbers of such cells present in the bone marrow of mice alive at the 16 week endpoint across treatments (FIG. 5D), with normal differentiation patterns also evidenced for lovastatic acid compared to DMSO-treated controls (FIG. 5E).

### Example 7

### Effects of BRD7116 and Lovastatic Acid on Primary Human CD34⁺ Leukemic and Normal Hematopoietic Cells

As compounds of therapeutic and biological interest require validation in human tissue, BRD7116 and lovastatic acid were tested in a series of primary, human cell assays. A CAFC assay was first performed using primary CD34⁺ cells isolated from normal human cord blood and CD34⁺ cells from six genetically distinct primary human leukemia samples (Table 2). The cells were treated with compound or DMSO carrier control at four doses (ranging from 1.25 μM to 10 μM) in triplicate for 18 hours, washed thoroughly, then plated onto human stromal MS-5 monolayers (Itoh, K., Tezuka, H., Sakoda, H., Konno, M., Nagata, K., Uchiyama, T., Uchino, H., and Mori, K. J. (1989). Exp Hematol 17, 145-153.) and maintained in coculture with one subsequent half media change. After 5 weeks (2 weeks for the FLT3-ITD sample (Moore, M. A., Dorn, D. C., Schuring a, J. J., Chung, K. Y., and Morrone, G. (2007). Exp Hematol 35, 105-116.)), cobblestone formation was determined, and dose-response curves against the 7 cell types were generated for each compound. Notably, this setup of pulse pretreatment for 18 hours in the absence of stroma probably underestimated the potency of these compounds, particularly in the case of BRD7116 which likely has non-cell-autonomous activity as discussed. Nevertheless, compared to DMSO controls, leukemia cobblestone inhibition was observed across all six primary leukemia samples tested for both BRD7116 and lovastatic acid (FIG. 6A). Strikingly, neither compound showed toxicity against the normal, primary CD34⁺ cells at any of the doses tested (FIG. 6B). The results of these human cobblestone studies are consistent with leukemia-selective activity at the stem cell level, and mirror our findings from the murine coculture system.

Finally, experiments were performed to examine whether BRD7116 or lovastatic acid were likely to cause myelosuppression, a type of deleterious toxicity in patients resulting from harm to normal human hematopoietic progenitor cells. This consideration is especially important as conventional therapies currently used in the clinic have myelosuppressive toxicities that are dose-limiting. To this end, a hematopoietic progenitor assay was employed to compare the effects of BRD7116 and lovastatic acid to effects given by such known myelosuppressive compounds. In this assay, normal primary human CD34⁺ cord blood cells were plated with cytokines in suspension, treated with compound for 7 days, and then examined for viability relative to DMSO treated controls. Daunorubicin and Ara-C, two conventional chemotherapeutics and frontline AML treatments, displayed toxicity to the hematopoietic progenitor cells (FIGS. 6C and 6D), consistent with their known myelosuppressive effects in the clinic. In contrast, both BRD7116 and lovastatic acid exhibited minimal toxicity after the 7 days of exposure (FIGS. 6E and 6F) even at concentrations found to selectively inhibit primary human leukemia cobblestone formation after just 18 hours of exposure.

TABLE 2
AML Patient Characteristics

| Sam- | Cytogenetic | | Primary or Therapy- |
|---|---|---|---|
| ple | Abnormalities | Gene Mutations* | Related |
| A | 7q-, trisomy 8 | none of examined | therapy-related |
| B | none | FLT3-ITD+ | primary |
| C | none | NPM1+ | primary |
| D | CBFB-MYH11 fusion | none of examined | primary |
| E | trisomy 11, del(17p) | none of examined | therapy-related |
| F | none | CEPBa double | primary |
| | | mutation | |

*examined: FLT3, NPM1, CEPBa, KIT
Clinical information is shown for each human sample, including genetic abnormalities detected and sample types.

### Example 8

### In vitro Therapeutic Index for BRD7116 and Lovastatic Acid Compared to Clinical Standard of Care

The therapeutic potential of these compounds was assessed further, side by side with current AML standard-of-care treatments. An in vitro therapeutic index was determined for each compound, a comparative measure of the potential for anti-LSC efficacy relative to the potential for dose-limiting toxicities to normal HSCs (myeloablation), or to normal progenitors (myelosuppression). Specifically, ratios were created using the IC50s for primary murine HSPCs in triple coculture as the numerator, and the IC50s for primary murine leukemia inhibition in triple coculture as the denominator, toward assessing the potential for HSC harm relative to LSC inhibition. Similarly, ratios were created using the IC50s for the primary human CD34+ progenitor assays (FIG. 6C-F) as numerator, and again the IC50s for the primary murine leukemia inhibition in triple coculture as the denominator, toward assessing the potential for normal hematopoietic progenitor cells harm relative to LSC inhibition. Note that this metric removes the factor of potency from the comparison, a factor which chemical optimization in the context of pre-clinical studies may improve (Guzman, M. L., Rossi, R. M., Neelakantan, S., Li, X., Corbett, C. A., Hassane, D. C., Becker, M. W., Bennett, J. M., Sullivan, E., Lachowicz, J. L., et al. (2007). Blood 110, 4427-4435.).

The preliminary ratios are shown in Table 3. For both types of ratios, a value as high as possible is desirable. Relative to Ara-C, a therapeutic known to have limited efficacy against the LSC subpopulation of human leukemias relative to the overall AML population (Guzman, M. L., Neering, S. J., Upchurch, D., Grimes, B., Howard, D. S., Rizzieri, D. A., Luger, S. M., and Jordan, C. T. (2001). Blood 98, 2301-2307.), as well as myelosuppressive toxicity effects, both BRD7116 and lovastatic acid yield an in vitro therapeutic index reflecting an improvement relative to these limitations. While in these assays, daunorubicin shows strong efficacy to LSC cobblestone inhibition, its therapeutic index is limited by toxicity to normal progenitor cells, a toxicity not observed for BRD7116 or lovastatic acid as discussed (FIG. 6C-F). To the extent that these assays are predictive, these results indicate that the assays described herein can reveal compounds that likely increase the therapeutic window relative to conventional cytotoxics currently in use in the clinic, providing the motivation for additional, larger scale screens using the present approach.

In aggregate, the studies described here identify promising small molecules with the potential to selectively perturb leukemia stem cell dependencies in the context of the bone marrow niche. These studies also demonstrate that the scope of therapeutic discovery is expanded when biological complexity is embraced at scale.

TABLE 3
In Vitro Therapeutic Index for BRD7116, Lovastatic Acid, and Standard of Care

| | MLL-AF9 | Normal | Normal | In vitro | In vitro |
|---|---|---|---|---|---|
| | LSC* | HSPC* | Progenitor* | Therapeutic Index | Therapeutic Index |
| Compound | IC₅₀ (nM) | IC₅₀ (nM) | IC₅₀ (nM) | (HSPC/LSC) | (Progenitors/LSC) |
| Ara-C | 2,500 | 5,000 | 2,500 | <2 | 1 |
| (standard of care) | | | | | |
| Daunorubicin | 2.5 | 50 | 20 | <20 | <8 |
| (standard of care) | | | | | |
| Lovastatic acid | 100 | 800 | 10,000 | >8 | >20 |
| BRD7116 | 500 | 5,000 | 10,000 | >10 | 100 |

A first comparison of the selectivity of the highlighted compounds to that of the standard of care is shown, as an estimated in vitro therapeutic index. The numerator is either the IC₅₀ for normal murine HPSCs treated in triple coculture (with murine LSCs on primary MSCs), toward addressing potential therapeutic benefit relative to the potential for myelotoxicity, or the IC₅₀ for normal human hematopoietic progenitors (shown in FIG. 6C-F), toward addressing potential therapeutic benefit relative to the potential for myelosupression. The denominator is the murine LSC effects in triple coculture with HSPCs on MSCs. For both types of indices, a value as high as possible above 1 is ideal.

### Example 9

### Effects of Selected Benzimidazole Hits on Primary Human CD34+ Leukemic Cells and Normal Hematopoietic Cells

As compounds of therapeutic and biological interest require validation in human tissue, a number of benzimidazole compounds including parbendazole and methiazole were tested in a series of primary, human cell assays. A CAFC assay was first performed using primary CD34⁺ cells isolated from normal human cord blood and CD34⁺ cells from six genetically distinct primary human leukemia samples (see Table 2 above). The cells were treated with compound or DMSO carrier control at four doses (ranging from 1.25 μM to 10 μM) in triplicate for 18 hours, washed thoroughly, then plated onto human stromal MS-5 monolayers (Itoh, K., Tezuka, H., Sakoda, H., Konno, M., Nagata, K., Uchiyama, T., Uchino, H., and Mori, K. J. (1989). Exp Hematol 17, 145-153.) and maintained in coculture with one subsequent half media change. After 5 weeks (2 weeks for the FLT3-ITD sample (Moore, M. A., Dorn, D. C., Schuring a, J. J., Chung, K. Y., and Morrone, G. (2007). Exp Hematol 35, 105-116.)), cobblestone formation was determined, and dose-response curves against the 7 cell types were generated for each compound. Compared to DMSO controls, leukemia cobblestone inhibition was observed across all six primary leukemia samples tested for both benzimidazole (FIG. 13A). In addition, neither compound showed toxicity against the normal, primary CD34⁺ cells at any of the doses tested (FIG. 13B).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for treating leukemia in a patient, the method comprising:
identifying the patient as being in remission; and
administering to the patient one or more of:
(a) a therapeutically effective amount of one or more statins, or a prodrug, acid, or form thereof,
(b) a therapeutically effective amount of a compound of formula (I): 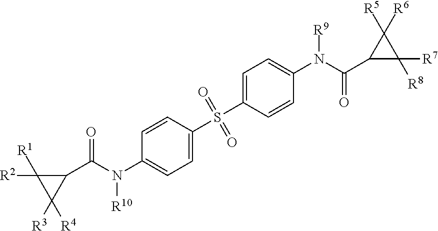
wherein:
R¹, R², R³, R⁴, R⁵, R⁶, R⁷, and R⁸ are independently selected from the group consisting of: hydrogen, C₁₋₆ alkyl, C₁₋₆ alkenyl, and C₁₋₆ alkynyl; and
R⁹ and R¹⁰ are independently selected from the group consisting of: hydrogen and C₁₋₆ alkyl
or a pharmaceutically acceptable salt form thereof;
(c) a therapeutically effective amount of a compound of formula (II): 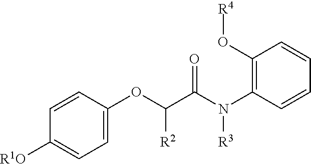
wherein:
R¹, R³, and R⁴ are independently selected from the group consisting of: hydrogen and C₁₋₆ alkyl; and
R² is selected from the group consisting of: hydrogen, C₁₋₆ alkyl, C₁₋₆ alkenyl, and C₁₋₆ alkynyl
or a pharmaceutically acceptable salt form thereof;
(d) a therapeutically effective amount of a compound of formula (III): 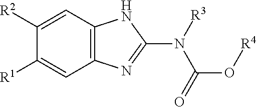
or a pharmaceutically acceptable salt form thereof,
wherein:
R¹ and R² are independently selected from the group consisting of hydrogen, C₁₋₆ alkyl, C₁₋₆ alkenyl, C₁₋₆ alkynyl, OR⁵, C(O)R⁵, SR⁶, S(O)₂R⁵, carbocyclyl, heterocyclyl, aryl, and heteroaryl;
R³ and R⁴ are independently selected from the group consisting of: hydrogen and C₁₋₆ alkyl; and
each R⁵ is independently selected from the group consisting of: hydrogen, C₁₋₆ alkyl, C₁₋₆ alkenyl, C₁₋₆ alkynyl, carbocyclyl, heterocyclyl, aryl, and heteroaryl;
(e) a therapeutically effective amount of a compound of formula (IV): 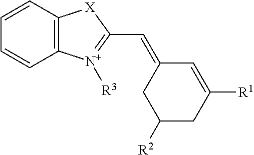
or a pharmaceutically acceptable salt form thereof,
wherein:
R¹ and R² are independently selected from the group consisting of: hydrogen, C₁₋₆ alkyl, C₁₋₆ alkenyl, and C₁₋₆ alkynyl; and
R³ is selected from the group consisting of: hydrogen and C₁₋₆ alkyl;
(f) a therapeutically effective amount of a compound of formula (V): 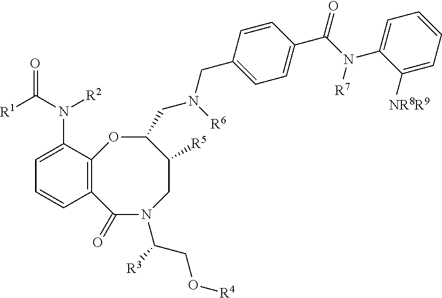
or a pharmaceutically acceptable salt form thereof,
wherein:
R¹ is selected from the group consisting of hydrogen, C₁₋₆ alkyl, C₁₋₆ alkenyl, C₁₋₆ alkynyl, NR¹⁰R¹¹, carbocyclyl, heterocyclyl, aryl, and heteroaryl;
R³ and R⁵ are independently selected from the group consisting of: hydrogen, C₁₋₆ alkyl, C₁₋₆ alkenyl, and C₁₋₆ alkynyl;
R², R⁴, R⁶, R⁷, R⁸, and R⁹ are independently selected from the group consisting of: hydrogen and C₁₋₆ alkyl; and
R¹⁰ and R¹¹ are independently selected from the group consisting of: hydrogen, C₁₋₆ alkyl, C₁₋₆ alkenyl, C₁₋₆ alkynyl, carbocyclyl, heterocyclyl, aryl, and heteroaryl;
(g) a therapeutically effective amount of a compound of formula (VI): 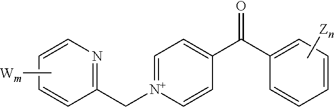
or a pharmaceutically acceptable salt form thereof,
wherein:
W and Z are independently selected from the group consisting of: halogen, OR¹, NR¹R², CN, NO₂, C₁₋₆ alkyl, C₁₋₆ alkenyl, and C₁₋₆ alkynyl;
R¹ and R² are independently selected from the group consisting of: hydrogen and C₁₋₆ alkyl;
m is an integer from 0 to 4; and
n is an integer from 0 to 5;
(h) a therapeutically effective amount of a compound of formula (VII): 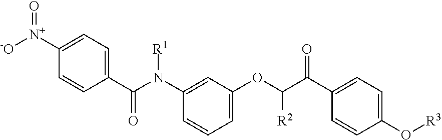
or a pharmaceutically acceptable salt form thereof,
wherein:
R¹ and R³ are independently selected from the group consisting of: hydrogen and C₁₋₆ alkyl; and
R² is selected from the group consisting of: hydrogen, C₁₋₆ alkyl, C₁₋₆ alkenyl, and C₁₋₆ alkynyl; or
(i) a therapeutically effective amount of a compound of formula (VIII): 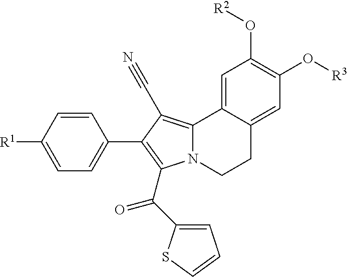
or a pharmaceutically acceptable salt form thereof,
wherein:
R¹ is selected from the group consisting of: hydrogen, C₁₋₆ alkyl, C₁₋₆ alkenyl, and C₁₋₆ alkynyl; and
R² and R³ are independently selected from the group consisting of: hydrogen and C₁₋₆ alkyl.

2. A method for treating leukemia in a patient, the method comprising: administering to the patient one or more of:
(j) a therapeutically effective amount of one or more statins, or a prodrug, acid, or form thereof,
(k) a therapeutically effective amount of a compound of formula (I): 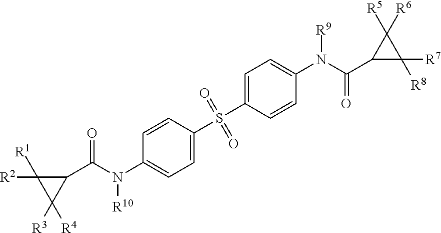
wherein:
R¹, R², R³, R⁴, R⁵, R⁶, R⁷, and R⁸ are independently selected from the group consisting of: hydrogen, C₁₋₆ alkyl, C₁₋₆ alkenyl, and C₁₋₆ alkynyl; and
R⁹ and R¹⁰ are independently selected from the group consisting of: hydrogen and C₁₋₆ alkyl
or a pharmaceutically acceptable salt form thereof;
(l) a therapeutically effective amount of a compound of formula (II): 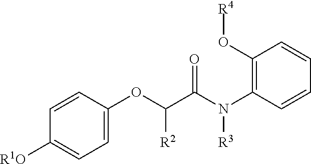
wherein:
R¹, R³, and R⁴ are independently selected from the group consisting of: hydrogen and C₁₋₆ alkyl; and
R² is selected from the group consisting of: hydrogen, C₁₋₆ alkyl, C₁₋₆ alkenyl, and C₁₋₆ alkynyl
or a pharmaceutically acceptable salt form thereof;
(m) a therapeutically effective amount of a compound of formula (III): 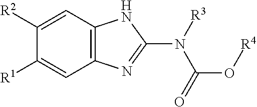
or a pharmaceutically acceptable salt form thereof,
wherein:
R¹ and R² are independently selected from the group consisting of hydrogen, C₁₋₆ alkyl, C₁₋₆ alkenyl, C₁₋₆ alkynyl, OR⁵, C(O)R⁵, SR⁶, S(O)₂R⁵, carbocyclyl, heterocyclyl, aryl, and heteroaryl;
R³ and R⁴ are independently selected from the group consisting of: hydrogen and C₁₋₆ alkyl; and
each R⁵ is independently selected from the group consisting of: hydrogen, C₁₋₆ alkyl, C₁₋₆ alkenyl, C₁₋₆ alkynyl, carbocyclyl, heterocyclyl, aryl, and heteroaryl;
(n) a therapeutically effective amount of a compound of formula (IV): 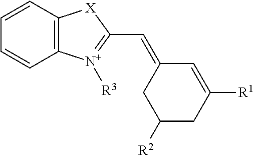
or a pharmaceutically acceptable salt form thereof,
wherein:
R¹ and R² are independently selected from the group consisting of: hydrogen, C₁₋₆ alkyl, C₁₋₆ alkenyl, and C₁₋₆ alkynyl; and
R³ is selected from the group consisting of: hydrogen and C₁₋₆ alkyl;
(o) a therapeutically effective amount of a compound of formula (V): 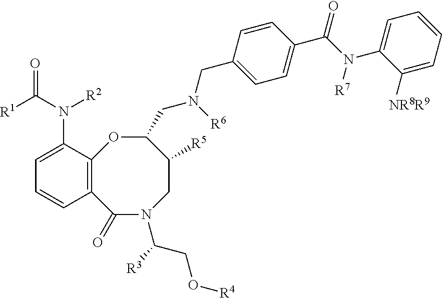
or a pharmaceutically acceptable salt form thereof,
wherein:
R¹ is selected from the group consisting of hydrogen, C₁₋₆ alkyl, C₁₋₆ alkenyl, C₁₋₆ alkynyl, NR¹⁰R¹¹, carbocyclyl, heterocyclyl, aryl, and heteroaryl;
R³ and R⁵ are independently selected from the group consisting of: hydrogen, C₁₋₆ alkyl, C₁₋₆ alkenyl, and C₁₋₆ alkynyl;
R², R⁴, R⁶, R⁷, R⁸, and R⁹ are independently selected from the group consisting of: hydrogen and C₁₋₆ alkyl; and
R¹⁰ and R¹¹ are independently selected from the group consisting of: hydrogen, C₁₋₆ alkyl, C₁₋₆ alkenyl, C₁₋₆ alkynyl, carbocyclyl, heterocyclyl, aryl, and heteroaryl;
(p) a therapeutically effective amount of a compound of formula (VI): 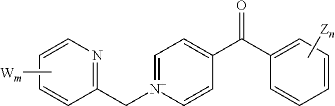
or a pharmaceutically acceptable salt form thereof,
wherein:
W and Z are independently selected from the group consisting of: halogen, OR¹, NR¹R², CN, NO₂, C₁₋₆ alkyl, C₁₋₆ alkenyl, and C₁₋₆ alkynyl;
R¹ and R² are independently selected from the group consisting of: hydrogen and C₁₋₆ alkyl;
m is an integer from 0 to 4; and
n is an integer from 0 to 5;
(q) a therapeutically effective amount of a compound of formula (VII): 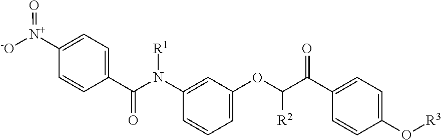
or a pharmaceutically acceptable salt form thereof,
wherein:
R¹ and R³ are independently selected from the group consisting of: hydrogen and C₁₋₆ alkyl; and
R² is selected from the group consisting of: hydrogen, C₁₋₆ alkyl, C₁₋₆ alkenyl, and C₁₋₆ alkynyl; or
(r) a therapeutically effective amount of a compound of formula (VIII): 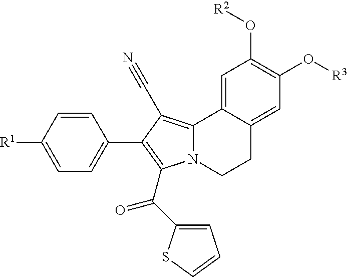
or a pharmaceutically acceptable salt form thereof,
wherein:
R¹ is selected from the group consisting of: hydrogen, C₁₋₆ alkyl, C₁₋₆ alkenyl, and C₁₋₆ alkynyl; and
R² and R³ are independently selected from the group consisting of: hydrogen and C₁₋₆ alkyl.

3. The method of claim 2, wherein the method comprises and at least one additional treatment for leukemia.

4. The method of claim 3, wherein the at least one additional treatment for leukemia is selected from the group consisting of chemotherapy and irradiation.

5. The method of claim 1 or 2, wherein the one or more statins is selected from the group consisting of: fluvastatin, cerivastatin, simvastatin, and acid forms thereof.

6. The method of claim 5, wherein the one or more statins is selected from the group consisting of: fluvastatin, cerivastatin, simvastatin, and acid forms thereof.

7. The method of claim 1, wherein the leukemia is selected from the group consisting of: chronic eosinophilic leukemia (CEL), chronic neutrophilic leukemia (CNL), chronic myelogenous leukemia (CML), chronic myelomonocytic leukemia (CMML), hairy cell leukemia (HCL), and chronic lymphocytic leukemia (CLL); acute leukemias include acute myelogenous leukemia (AML) and acute lymphocytic leukemia (ALL).

8. The method of claim 2, wherein the leukemia is selected from the group consisting of: chronic eosinophilic leukemia (CEL), chronic neutrophilic leukemia (CNL), chronic myelogenous leukemia (CML), chronic myelomonocytic leukemia (CMML), hairy cell leukemia (HCL), and chronic lymphocytic leukemia (CLL); acute leukemias include acute myelogenous leukemia (AML) and acute lymphocytic leukemia (ALL).

9. The method of claim 1, further comprising administering at least one other treatment for leukemia to the patient.

10. The method of claim 8, wherein the at least one other treatment is selected from the group consisting of chemotherapy and irradiation.

11. The method of claim 9, wherein the chemotherapy comprises administration of an agent selected from the group consisting of abarelix, aldesleukin, alemtuzumab, alitretinoin, allopurinol, altretamine, anastrozole, arsenic trioxide, asparaginase, azacitidine, bevacizumab, bexarotene, bleomycin, bortezombi, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, epirubicin, erlotinib, estramustine, etoposide phosphate, etoposide, exemestane, fentanyl citrate, filgrastim, floxuridine, fludarabine, fluorouracil, fulvestrant, gefitinib, gemcitabine, gemtuzumab ozogamicin, goserelin acetate, histrelin acetate, ibritumomab tiuxetan, idarubicin, ifosfamide, imatinib mesylate, interferon alfa 2a, irinotecan, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, oxaliplatin, paclitaxel, pamidronate, panitumumab, pegaspargase, pegfilgrastim, pemetrexed disodium, pentostatin, pipobroman, plicamycin, procarbazine, quinacrine, rasburicase, rituximab, sorafenib, streptozocin, sunitinib, sunitinib maleate, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, vorinostat, and zoledronate.

12. The method of claim 3, wherein the chemotherapy comprises administration of an agent selected from the group consisting of abarelix, aldesleukin, alemtuzumab, alitretinoin, allopurinol, altretamine, anastrozole, arsenic trioxide, asparaginase, azacitidine, bevacizumab, bexarotene, bleomycin, bortezombi, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, epirubicin, erlotinib, estramustine, etoposide phosphate, etoposide, exemestane, fentanyl citrate, filgrastim, floxuridine, fludarabine, fluorouracil, fulvestrant, gefitinib, gemcitabine, gemtuzumab ozogamicin, goserelin acetate, histrelin acetate, ibritumomab tiuxetan, idarubicin, ifosfamide, imatinib mesylate, interferon alfa 2a, irinotecan, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, oxaliplatin, paclitaxel, pamidronate, panitumumab, pegaspargase, pegfilgrastim, pemetrexed disodium, pentostatin, pipobroman, plicamycin, procarbazine, quinacrine, rasburicase, rituximab, sorafenib, streptozocin, sunitinib, sunitinib maleate, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, vorinostat, and zoledronate.

13. A method of identifying a candidate compound for the treatment of leukemia, the method comprising:
(a) providing a test sample comprising a co-culture of stromal cells and primary leukemic hematopoietic cells;
(b) contacting the test sample with a test compound, and maintaining the co-culture for a time and under conditions sufficient for the primary leukemic hematopoietic cells to form areas of cobblestoning;
(c) obtaining one or more images of the test sample;
(d) detecting areas of cobblestoning in the images of the test sample by applying a classifier to the images, wherein the classifier comprises a set of rules that are executable to identify areas of cobblestoning; and
(e) comparing the areas of cobblestoning in a test sample in the presence of the test compound to areas of cobblestoning in a test sample in the absence of the test compound, and
(f) selecting as a candidate compound a test compound that reduces areas of cobblestoning.

14. The method of claim 13, wherein providing the co-culture comprises:
plating a population of stromal cells in a culture dish; and
adding a population of primary hematopoietic stem cells in the same culture dish.

15. The method of claim 13, further comprising:
(g) providing a control sample comprising a co-culture of stromal cells and normal primary hematopoietic cells;
(h) contacting the control sample with a test compound, and maintaining the co-culture for a time and under conditions sufficient for the normal hematopoietic cells to form areas of cobblestoning;
(i) obtaining one or more images of the control sample;
(j) detecting areas of cobblestoning in the images of the control sample by applying the classifier to the images of the control sample; and
(k) comparing the areas of cobblestoning in a control sample in the presence of the test compound to areas of cobblestoning in a control sample in the absence of the test compound, and
(l) selecting as a candidate compound a test compound that reduces areas of cobblestoning in the test sample but does not reduce areas of cobblestoning in the control sample.

16. The method of claim 13, wherein the primary leukemic hematopoietic cells are enriched for leukemic stem cells.

17. The method of claim 13, wherein the stromal cells are primary cells or from an immortalized cell line.

18. The method of claim 13, wherein the classifier comprises a set of rules that are executable to identify cobblestoning in an item of data.

19. The method of claim 18, wherein the rules comprise one or more of: Cell objects that that have greater than a selected percentage of their perimeter touching other objects; Cell objects with low texture feature (Gabor wavelet) at a 3 pixel scale in the DsRed channel; Cell objects with fewer than a selected number of neighbor objects (within 2 pixels); Cell objects with low texture contrast at a 3 pixel scale in the DsRed channel; Cell objects with high minimum intensity in DsRed channel greater than a selected amount; Cell objects standard deviation in DsRed channel less than a selected amount; Cell objects with low minimum intensity in Stromal channel less than a selected amount; Cell objects with greater than a selected number of neighbor objects (within 2 pixels); Cell objects with a 9th order Zernike shape feature greater than a selected level; Cell objects with a low texture feature (Sum of Entropy) at a 1 pixel scale in the DsRed channel.
